SEQUENCE LISTING (94 sequences)

SEQ ID NO: 1 - DNA, 4536 bp, Artificial Sequence
  pTRIP CMV GFP
tggaagggctaattcactcccaacgaagacaagatatccttgatctgtggatctaccacacacaaggctacttccctgattagcagaactacacaccagggccagggatcagatatccactgacctttggatggtgctacaagctagtaccagttgagccagagaagttagaagaagccaacaaaggagagaacaccagcttgttacaacctgtgagcctgcatgggatggatgacccggagagagaagtgttagagtggaggtttgacagccgcctagcatttcatcacggtggcccgagagctgcatccggagtacttcaagaactgctgatatcgagcttgctacaagggactttccgctgggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgccgaattcacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccactttggctgatacgcgtggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgcgatcggatccccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagctcgagctcaagcttcgaattcccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattctgcagtcgacggtacctttaagaccaatgacttacaaggcagctgtagatcttagccactttttaaaagaaaaggggggactggaagggctaattcactcccaacgaagacaagatcgtcgagagatgctgcatataagcagctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagca

SEQ ID NO: 2 - DNA, 4468 bp, Artificial Sequence
  pTRIP B2M GFP
tggaagggctaattcactcccaacgaagacaagatatccttgatctgtggatctaccacacacaaggctacttccctgattagcagaactacacaccagggccagggatcagatatccactgacctttggatggtgctacaagctagtaccagttgagccagagaagttagaagaagccaacaaaggagagaacaccagcttgttacaacctgtgagcctgcatgggatggatgacccggagagagaagtgttagagtggaggtttgacagccgcctagcatttcatcacggtggcccgagagctgcatccggagtacttcaagaactgctgatatcgagcttgctacaagggactttccgctgggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgccgaattcacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccactttggctgatacgcgccggaaaccctgcagggaattccccagctgtagttataaacagaagttctccttctgctaggtagcattcaaagatcttaatcttctgggtttccgttttctcgaatgaaaaatgcaggtccgagcagttaactggcgggggcaccattagcaagtcacttagcatctctggggccagtctgcaaagcgagggggcagccttaatgtgcctccagcctgaagtcctagaatgagcgcccggtgtcccaagctggggcgcgcaccccagatcggagggcgccgatgtacagacagcaaactcacccagtctagtgcatgccttcttaaacatcacgagactctaagaaaaggaaactgaaaacgggaaagtccctctctctaacctggcactgcgtcgctggcttggagacaggtgacggtccctgcgggccttgtcctgattggctgggcacgcgtttaatataagtggaggcgtcgcgctggcgggcattcctgaagctgacagcattcgggccgagcgatcggatccccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagctcgagctcaagcttcgaattcccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattctgcagtcgacggtacctttaagaccaatgacttacaaggcagctgtagatcttagccactttttaaaagaaaaggggggactggaagggctaattcactcccaacgaagacaagatcgtcgagagatgctgcatataagcagctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagca

SEQ ID NO: 3 - DNA, 11904 bp, Artificial Sequence
  PACKAGING 8.74 PLASMID
gggctgcaggaattaattcgagctcgcccgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgcgttgacgcgcacggcaagaggcgaggggcggcgactggtgagagatgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcagctgacacaggacacagcaatcaggtcagccaaaattaccctatagtgcagaacatccaggggcaaatggtacatcaggccatatcacctagaactttaaatgcatgggtaaaagtagtagaagagaaggctttcagcccagaagtgatacccatgttttcagcattatcagaaggagccaccccacaagatttaaacaccatgctaaacacagtggggggacatcaagcagccatgcaaatgttaaaagagaccatcaatgaggaagctgcagaatgggatagagtgcatccagtgcatgcagggcctattgcaccaggccagatgagagaaccaaggggaagtgacatagcaggaactactagtacccttcaggaacaaataggatggatgacacataatccacctatcccagtaggagaaatctataaaagatggataatcctgggattaaataaaatagtaagaatgtatagccctaccagcattctggacataagacaaggaccaaaggaaccctttagagactatgtagaccgattctataaaactctaagagccgagcaagcttcacaagaggtaaaaaattggatgacagaaaccttgttggtccaaaatgcgaacccagattgtaagactattttaaaagcattgggaccaggagcgacactagaagaaatgatgacagcatgtcagggagtggggggacccggccataaagcaagagttttggctgaagcaatgagccaagtaacaaatccagctaccataatgatacagaaaggcaattttaggaaccaaagaaagactgttaagtgtttcaattgtggcaaagaagggcacatagccaaaaattgcagggcccctaggaaaaagggctgttggaaatgtggaaaggaaggacaccaaatgaaagattgtactgagagacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataaagataggggggcaattaaaggaagctctattagatacaggagcagatgatacagtattagaagaaatgaatttgccaggaagatggaaaccaaaaatgatagggggaattggaggttttatcaaagtaagacagtatgatcagatactcatagaaatctgcggacataaagctataggtacagtattagtaggacctacacctgtcaacataattggaagaaatctgttgactcagattggctgcactttaaattttcccattagtcctattgagactgtaccagtaaaattaaagccaggaatggatggcccaaaagttaaacaatggccattgacagaagaaaaaataaaagcattagtagaaatttgtacagaaatggaaaaggaaggaaaaatttcaaaaattgggcctgaaaatccatacaatactccagtatttgccataaagaaaaaagacagtactaaatggagaaaattagtagatttcagagaacttaataagagaactcaagatttctgggaagttcaattaggaataccacatcctgcagggttaaaacagaaaaaatcagtaacagtactggatgtgggcgatgcatatttttcagttcccttagataaagacttcaggaagtatactgcatttaccatacctagtataaacaatgagacaccagggattagatatcagtacaatgtgcttccacagggatggaaaggatcaccagcaatattccagtgtagcatgacaaaaatcttagagccttttagaaaacaaaatccagacatagtcatctatcaatacatggatgatttgtatgtaggatctgacttagaaatagggcagcatagaacaaaaatagaggaactgagacaacatctgttgaggtggggatttaccacaccagacaaaaaacatcagaaagaacctccattcctttggatgggttatgaactccatcctgataaatggacagtacagcctatagtgctgccagaaaaggacagctggactgtcaatgacatacagaaattagtgggaaaattgaattgggcaagtcagatttatgcagggattaaagtaaggcaattatgtaaacttcttaggggaaccaaagcactaacagaagtagtaccactaacagaagaagcagagctagaactggcagaaaacagggagattctaaaagaaccggtacatggagtgtattatgacccatcaaaagacttaatagcagaaatacagaagcaggggcaaggccaatggacatatcaaatttatcaagagccatttaaaaatctgaaaacaggaaagtatgcaagaatgaagggtgcccacactaatgatgtgaaacaattaacagaggcagtacaaaaaatagccacagaaagcatagtaatatggggaaagactcctaaatttaaattacccatacaaaaggaaacatgggaagcatggtggacagagtattggcaagccacctggattcctgagtgggagtttgtcaatacccctcccttagtgaagttatggtaccagttagagaaagaacccataataggagcagaaactttctatgtagatggggcagccaatagggaaactaaattaggaaaagcaggatatgtaactgacagaggaagacaaaaagttgtccccctaacggacacaacaaatcagaagactgagttacaagcaattcatctagctttgcaggattcgggattagaagtaaacatagtgacagactcacaatatgcattgggaatcattcaagcacaaccagataagagtgaatcagagttagtcagtcaaataatagagcagttaataaaaaaggaaaaagtctacctggcatgggtaccagcacacaaaggaattggaggaaatgaacaagtagataaattggtcagtgctggaatcaggaaagtactatttttagatggaatagataaggcccaagaagaacatgagaaatatcacagtaattggagagcaatggctagtgattttaacctaccacctgtagtagcaaaagaaatagtagccagctgtgataaatgtcagctaaaaggggaagccatgcatggacaagtagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggaaaggaccagcaaagctcctctggaaaggtgaaggggcagtagtaatacaagataatagtgacataaaagtagtgccaagaagaaaagcaaagatcatcagggattatggaaaacagatggcaggtgatgattgtgtggcaagtagacaggatgaggattaacacatggaattctgcaacaactgctgtttatccatttcagaattgggtgtcgacatagcagaataggcgttactcgacagaggagagcaagaaatggagccagtagatcctagactagagccctggaagcatccaggaagtcagcctaaaactgcttgtaccaattgctattgtaaaaagtgttgctttcattgccaagtttgtttcatgacaaaagccttaggcatctcctatggcaggaagaagcggagacagcgacgaagagctcatcagaacagtcagactcatcaagcttctctatcaaagcagtaagtagtacatgtaatgcaacctataatagtagcaatagtagcattagtagtagcaataataatagcaatagttgtgtggtccatagtaatcatagaatataggaaaatggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatccttggcacttatctgggacgatctgcggagcctgtgcctcttcagctaccaccgcttgagagacttactcttgattgtaacgaggattgtggaacttctgggacgcagggggtgggaagccctcaaatattggtggaatctcctacaatattggagtcaggagctaaagaatagtgctgttagcttgctcaatgccacagccatagcagtagctgaggggacagatagggttatagaagtagtacaaggagcttgtagagctattcgccacatacctagaagaataagacagggcttggaaaggattttgctataagctcgagtgaccttcagaccttggcactggaggtggcccggcagaagcgcggcatcgtggatcagtgctgcaccagcatctgctctctctaccaactggagaactactgcaactaggcccaccactaccctgtccacccctctgcaatgaataaaacctttgaaagagcactacaagttgtgtgtacatgcgtgcatgtgcatatgtggtgcggggggaacatgagtggggctggctggagtggcgatgataagctgtcaaacatgagaattaattcttgaagacgaaagggcctcgtgatacgcctatttttataggttaatgtcatgataataatggtttcttagtctagaattaattccgtgtattctatagtgtcacctaaatcgtatgtgtatgatacataaggttatgtattaattgtagccgcgttctaacgacaatatgtacaagcctaattgtgtagcatctggcttactgaagcagaccctatcatctctctcgtaaactgccgtcagagtcggtttggttggacgaaccttctgagtttctggtaacgccgtcccgcacccggaaatggtcagcgaaccaatcagcagggtcatcgctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttggacacaagacaggcttgcgagatatgtttgagaataccactttatcccgcgtcagggagaggcagtgcgtaaaaagacgcggactcatgtgaaatactggtttttagtgcgccagatctctataatctcgcgcaacctattttcccctcgaacactttttaagccgtagataaacaggctgggacacttcacatgagcgaaaaatacatcgtcacctgggacatgttgcagatccatgcacgtaaactcgcaagccgactgatgccttctgaacaatggaaaggcattattgccgtaagccgtggcggtctgtaccgggtgcgttactggcgcgtgaactgggtattcgtcatgtcgataccgtttgtatttccagctacgatcacgacaaccagcgcgagcttaaagtgctgaaacgcgcagaaggcgatggcgaaggcttcatcgttattgatgacctggtggataccggtggtactgcggttgcgattcgtgaaatgtatccaaaagcgcactttgtcaccatcttcgcaaaaccggctggtcgtccgctggttgatgactatgttgttgatatcccgcaagatacctggattgaacagccgtgggatatgggcgtcgtattcgtcccgccaatctccggtcgctaatcttttcaacgcctggcactgccgggcgttgttctttttaacttcaggcgggttacaatagtttccagtaagtattctggaggctgcatccatgacacaggcaaacctgagcgaaaccctgttcaaaccccgctttaaacatcctgaaacctcgacgctagtccgccgctttaatcacggcgcacaaccgcctgtgcagtcggcccttgatggtaaaaccatccctcactggtatcgcatgattaaccgtctgatgtggatctggcgcggcattgacccacgcgaaatcctcgacgtccaggcacgtattgtgatgagcgatgccgaacgtaccgacgatgatttatacgatacggtgattggctaccgtggcggcaactggatttatgagtgggccccggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgtt

SEQ ID NO: 4 - DNA, 5140 bp, Artificial Sequence
  pCMV-VSV-INDco
ctggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttcttgactcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatcctgatcagccaccatgaaatgcctgctctatctggccttcctgttcatcggcgtgaactgcaagttcaccatcgtgttcccccacaaccagaagggcaactggaagaacgtgcccagcaactaccactactgccccagcagcagcgacctgaactggcacaacgacctgatcggcaccgccctgcaggtgaagatgcccaagagccacaaggccattcaggctgatggctggatgtgtcatgccagcaagtgggtgaccacctgcgacttccggtggtacggccccaagtacatcacccacagcatccgcagcttcacccccagcgtggagcagtgcaaagagagcatcgagcagaccaagcagggcacctggctgaaccccggcttccccccccagtcctgcggctacgccaccgtgaccgacgccgaggccgtgatcgtgcaggtgaccccccaccacgtgctggtcgacgagtacaccggcgagtgggtggacagccagttcatcaacggcaagtgcagcaactacatctgccctaccgtgcacaacagcaccacctggcacagcgactacaaggtgaaaggcctgtgcgacagcaacctgatcagcatggacatcacctttttcagcgaggacggcgagctgtccagcctgggcaaagagggcaccggcttcagaagcaactacttcgcctacgagacaggcggcaaggcctgcaagatgcagtactgcaagcactggggcgtgcggctgcctagcggcgtgtggttcgagatggccgacaaggacctgttcgccgctgcccggttccctgagtgccccgagggcagcagcatcagcgcccccagccagaccagcgtggacgtgagcctgatccaggacgtggagagaatcctggactacagcctgtgccaggaaacctggtccaagatcagagccggcctgcccatcagccccgtggacctgagctacctggcccccaagaaccccggcaccggcccagccttcaccatcatcaatggcaccctgaagtacttcgagacacggtacatcagagtggacattgccgcccctatcctgagccggatggtgggcatgatcagcggcaccaccaccgagcgggagctgtgggacgactgggccccctacgaggatgtggagatcggccccaacggcgtgctgcggaccagcagcggctacaagttccccctgtacatgatcggccacggcatgctggacagcgacctgcacctgagcagcaaggcccaggtgttcgagcacccccacatccaggacgccgccagccagctgcccgacgacgagagcctgttcttcggcgacaccggcctgagcaagaaccccatcgaactggtggagggctggttcagcagctggaagagcagcattgccagctttttcttcatcatcggcctgatcatcgggctgtttctggtgctgagagtgggcatccacctgtgcatcaagctgaagcacaccaagaagcggcagatctacaccgacatcgagatgaatcgcctggggaagtaagaattctgcagatatccagcacagtggcggccgctcgagtgtacaaattcccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatcgctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaa

SEQ ID NO: 5 - DNA, 5158 bp, Artificial Sequence
  pCMV-VSV-Njco
ctggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttcttgactcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatcctgatcagccaccatgctgtcatatctgatctttgccctggctgtgagcccaatcctcggaaagatcgaaatcgtgttcccacaacacaccacaggggactggaagcgcgtgccccacgagtacaactactgcccgacctcagccgacaagaatagccacggcacgcagaccggcatccctgtggagctgaccatgcccaaggggctcacaacgcaccaagtcgaaggcttcatgtgccacagcgctctctggatgacaacctgcgattttcgctggtatggccccaagtacatcacgcacagcatccacaatgaggaaccaaccgactaccagtgcctcgaagccatcaagtcatacaaggatggggtgagcttcaaccccggcttcccgccccaatcatgtggctacggcaccgtgaccgacgccgaggcccacatcgtgaccgtgacaccccactcagtcaaggtggacgagtacacaggcgaatggatcgacccccacttcatcgggggccgctgtaagggccaaatctgcgagaccgtgcacaacagcaccaagtggtttacgtcatcagacggcgaaagcgtgtgcagccaactgtttacgctcgtgggcggcatcttctttagcgacagcgaggagatcaccagcatgggcctcccggagacaggaatccgcagcaactactttccgtacatcagcaccgagggaatctgtaagatgcctttttgccgcaagcagggatataagctgaagaatgacctgtggttccagatcatggacccggacctggacaagaccgtccgcgatctgccccacatcaaggactgtgatctgtcatcaagcatcatcacccccggagaacacgccacggacatcagcctcatcagcgatgtggagcgcatcctcgactacgctctctgccagaacacatggagcaagatcgaaagcggcgaacccatcaccccagtggacctgagctatctcggcccaaagaaccccggcgtggggcccgtgttcaccatcatcaacgggagcctgcactactttacaagcaagtatctgcgcgtggagctcgaaagcccagtcatcccccgcatggaggggaaggtggccgggacccgcatcgtgcgccagctgtgggaccagtggttcccttttggcgaggtggaaatcggccccaacggcgtgctgaagaccaagcaaggatataagttcccgctgcacatcatcgggacgggcgaagtggacagcgatatcaagatggagcgcgtggtcaagcactgggagcacccacacatcgaggctgctcagacctttctcaagaaggacgataccggcgaagtcctgtattacggggatacgggagtgagcaagaaccctgtggagctggtggaaggctggttcagcggatggcgctcaagcctgatgggcgtgctggccgtcatcatcggatttgtgatcctgatgttcctcatcaagctgatcggcgtgctgtcaagcctgttccgccctaagcgccgcccaatctacaagagcgacgtcgagatggcccactttcgctaagaattctgcagatatccagcacagtggcggccgctcgagtgtacaaattcccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatcgctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaa

SEQ ID NO: 6 - DNA, 720 bp, Artificial Sequence
  eGFP
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 7 - protein, 239 aa, Artificial Sequence
  eGFP protein
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 8 - DNA, 1725 bp, Artificial Sequence
  A111-Luciferase
ggatccgccaccatggccgcagccgcagccgtgaagaactggatgactcaaacgctcgccgctgcaatggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaactcgag

SEQ ID NO: 9 - protein, 568 aa, Artificial Sequence
  A111-Luciferase
MAAAAAVKNWMTQTLAAAMEDAKNIKKGPAPFYPLEDGTAGEQLHKAMKRYALVPGTIAFTDAHIEVDITYAEYFEMSVRLAEAMKRYGLNTNHRIVVCSENSLQFFMPVLGALFIGVAVAPANDIYNERELLNSMGISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKIAV

SEQ ID NO: 10 - DNA, 1026 bp, Plasmodium berghei
  misc_feature: circumsporozoite (CS) protein (CSP)
atggccaagaagtgtaccatactggtcgttgcgtcacttctgttggtcaattctctgctcccaggctatggacagaacaaatccattcaggcacagaggaacctcaacgaactctgctacaatgaagggaatgacaataagctgtatcacgtgctgaattccaagaacggcaaaatctacaatcgcaacacagtaaatcggttgcttgccgatgcacccgagggtaagaagaatgaaaagaagaatgagaaaatcgagcgcaacaacaagcttaaacagccaccgcctcctcctaacccaaatgacccaccgccacccaatccaaacgacccaccgcctcccaaccctaacgatcctccaccgcccaaccctaatgacccaccacctcccaatgcaaacgatccaccccctcctaacgctaacgaccctgctccacccaacgctaacgatcccgcgccccccaatgccaacgaccccgcaccacctaatgccaacgatcccgccccgcccaatgctaatgatcctccaccacccaacccaaacgaccctgcccctcctaatgctaacgatccaccacctcccaatccgaatgatcccgctccacctcaggggaacaacaaccctcagccccaacctagaccacagccgcagccccaaccccaaccccagccccagcctcaaccccagccccagccacgtccgcagcctcagcctcaacctggaggcaacaataacaacaagaataacaacaacgacgacagctacattccaagtgccgagaaaattctggagtttgttaagcagatccgagacagcataaccgaagaatggtcacagtgtaacgtgacgtgtggatctggcatcagagtgaggaaacggaagggttccaataagaaagcagaggatctgactctggaggacattgatacagagatctgcaaaatggacaaatgcagctctatcttcaacatcgtgagtaatagcctcgggtttgtgattctgctggtcctggtgttcttcaattga

SEQ ID NO: 11 - protein, 341 aa, Plasmodium berghei
  misc_feature: CSP
MAKKCTILVVASLLLVNSLLPGYGQNKSIQAQRNLNELCYNEGNDNKLYHVLNSKNGKIYNRNTVNRLLADAPEGKKNEKKNEKIERNNKLKQPPPPPNPNDPPPPNPNDPPPPNPNDPPPPNPNDPPPPNANDPPPPNANDPAPPNANDPAPPNANDPAPPNANDPAPPNANDPPPPNPNDPAPPNANDPPPPNPNDPAPPQGNNNPQPQPRPQPQPQPQPQPQPQPQPQPRPQPQPQPGGNNNNKNNNNDDSYIPSAEKILEFVKQIRDSITEEWSQCNVTCGSGIRVRKRKGSNKKAEDLTLEDIDTEICKMDKCSSIFNIVSNSLGFVILLVLVFFN

SEQ ID NO: 12 - protein, 340 aa, Plasmodium berghei
  misc_feature: CSP
MKKCTILVVASLLLVNSLLPGYGQNKSIQAQRNLNELCYNEGNDNKLYHVLNSKNGKIYNRNTVNRLLADAPEGKKNEKKNEKIERNNKLKQPPPPPNPNDPPPPNPNDPPPPNPNDPPPPNPNDPPPPNANDPPPPNANDPAPPNANDPAPPNANDPAPPNANDPAPPNANDPPPPNPNDPAPPNANDPPPPNPNDPAPPQGNNNPQPQPRPQPQPQPQPQPQPQPQPQPRPQPQPQPGGNNNNKNNNNDDSYIPSAEKILEFVKQIRDSITEEWSQCNVTCGSGIRVRKRKGSNKKAEDLTLEDIDTEICKMDKCSSIFNIVSNSLGFVILLVLVFFN

SEQ ID NO: 13 - DNA, 1203 bp, Artificial Sequence
  PfCSPhumanCO
gccaccatggctatgagaaagctcgcgatcctttccgtgagctcattcctgtttgtcgaagccttgttccaggagtatcagtgctatggatcatcctccaacacaagagtgctcaacgaactgaactatgacaatgccggtactaacctctacaatgaactggagatgaactactacggcaaacaggagaactggtattccctgaagaagaattccagatcactgggcgaaaacgacgatgggaataatgaggataacgagaagttgcggaaaccaaagcacaagaagttgaaacaacccgccgacggaaaccctgatcctaacgccaacccaaatgtagatcccaacgccaacccaaacgtcgatcccaatgccaatcccaatgttgaccccaatgcaaaccctaatgcaaatcccaatgccaatcccaatgcaaatcctaatgctaatccaaacgccaaccctaacgcgaaccccaacgccaatcctaacgcaaatcctaacgcaaatcctaatgccaaccctaatgcgaacccgaacgctaatcctaacgctaatccgaatgcaaatccaaatgcaaatccgaacgccaatcccaacgtagacccaaatgcaaacccgaacgctaacccgaacgcaaacccaaacgccaatccgaacgctaatcccaatgctaatcccaacgctaaccccaatgccaacccgaatgccaaccccaatgcgaatccaaatgcgaacccaaacgccaatccgaatgcgaatcctaacgctaacccaaatgctaatccaaacgctaatccaaatgcgaaccccaatgcgaatccaaataagaacaatcaggggaatggtcagggccataacatgcctaacgaccccaaccgaaatgtggacgagaacgctaacgcaaacagcgctgtaaagaacaacaacaatgaggagccttctgacaagcacatcaaagagtacctgaataaaatccagaacagtctttctacggaatggtccccatgtagtgttacttgtggcaatgggattcaagtcaggatcaaaccaggctctgcgaataagcctaaggatgaactggattatgccaatgacatcgagaagaaaatatgcaagatggagaagtgcagtagcgtgttcaatgtcgtaaactcaagcataggtctgataatggtactgagctttctgttcctcaactaa

SEQ ID NO: 14 - protein, 398 aa, Artificial Sequence
  PfCSP
MAMRKLAILSVSSFLFVEALFQEYQCYGSSSNTRVLNELNYDNAGTNLYNELEMNYYGKQENWYSLKKNSRSLGENDDGNNEDNEKLRKPKHKKLKQPADGNPDPNANPNVDPNANPNVDPNANPNVDPNANPNANPNANPNANPNANPNANPNANPNANPNANPNANPNANPNANPNANPNANPNANPNANPNANPNVDPNANPNANPNANPNANPNANPNANPNANPNANPNANPNANPNANPNANPNANPNANPNANPNANPNANPNANPNKNNQGNGQGHNMPNDPNRNVDENANANSAVKNNNNEEPSDKHIKEYLNKIQNSLSTEWSPCSVTCGNGIQVRIKPGSANKPKDELDYANDIEKKICKMEKCSSVFNVVNSSIGLIMVLSFLFLN

SEQ ID NO: 15 - protein, 423 aa, Artificial Sequence
  CSP
MRKLAILSVSSFLFVEALFQEYQCYGSSSNTRVLNELNYDNAGTNLYNELEMNYYGKQENWYSLKKNSRSLGENDDGNNNNGDNGREGKDEDKRDGNNEDNEKLRKPKHKKLKQPGDGNPDPNANPNVDPNANPNVDPNANPNVDPNANPNANPNANPNANPNANPNANPNANPNANPNANPNANPNANPNVDPNANPNANPNANPNANPNANPNANPNANPNANPNANPNANPNANPNANPNANPNANPNANPNANPNANPNANPNANPNANPNANPNANPNANPNANPNANPNANPNKNNQGNGQGHNMPNDPNRNVDENANANNAVKNNNNEEPSDKHITEYLKKIQNSLSTEWSPCSVTCGNGIQVRIKPGSAGKSKDELDYENDIEKKICKMEKCSSVFNVVNSSIGLIMVLSFLFLN

SEQ ID NO: 16 - DNA, 1143 bp, Artificial Sequence
  PfCSPhumanCO
gccaccatggctaagaacttcattctgcttgccgtaagctctattctcttggtagacttgtttccaacacactgtggacataatgtagatctcagtaaagcaattaacctcaatggtgttaattttaataatgtagacgcatcctctctcggcgcggcacacgtgggtcaaagtgccagtagggggcgcggactcggcgagaacccggacgacgaagaaggggatgcgaaaaagaagaaggatgggaagaaggcagaacccaagaatccaagggagaataagctgaagcagccaggagatagagctgacggccagcctgccggcgatagggccgacggacaacccgcgggagaccgcgcggatggccaacctgcgggtgaccgcgctgacgggcagcccgcaggagacagagccgccggacaacccgccggcgaccgagcggatggtcagcccgcgggcgatcgagcggacggtcagccagctggagatcgcgcggacggacaacctgcgggggacagggctgacggtcaaccagccggtgatagagcagcgggccaaccggcaggggaccgagccgcagggcagcccgccggggacagggctgacgggcagccagcgggggatagggccgccggtcaacctgcaggcgatcgagctgacggccaacccgcgggtgaccgggcagccggccaacctgctggggatcgagcagacggacagccggccggagatcgggccgctggccaaccggctggagacagagccgctggtcaacctgccggagatcgggccgctgggcaggccgcgggtgacagggcagccggacaagcagcaggggggaacgccggtggacaaggacagaacaatgagggtgcgaatgccccgaatgaaaagagcgttaaagagtacctggataaggttagagcaaccgtaggcaccgaatggaccccctgctccgtgacttgcggggtaggtgttcgggtcaggcggagggtcaatgcggcgaacaagaaaccggaagatctgactttgaacgatcttgaaacagatgtatgcacgatggacaagtgtgctggcatatttaacgtagtcagtaactctctcggccttgtgatcctcttggtcttggcgctgtttaactag

SEQ ID NO: 17 - protein, 378 aa, Plasmodium vivax
  misc_feature: PvCSP
MAKNFILLAVSSILLVDLFPTHCGHNVDLSKAINLNGVNFNNVDASSLGAAHVGQSASRGRGLGENPDDEEGDAKKKKDGKKAEPKNPRENKLKQPGDRADGQPAGDRADGQPAGDRADGQPAGDRADGQPAGDRAAGQPAGDRADGQPAGDRADGQPAGDRADGQPAGDRADGQPAGDRAAGQPAGDRAAGQPAGDRADGQPAGDRAAGQPAGDRADGQPAGDRAAGQPAGDRADGQPAGDRAAGQPAGDRAAGQPAGDRAAGQAAGDRAAGQAAGGNAGGQGQNNEGANAPNEKSVKEYLDKVRATVGTEWTPCSVTCGVGVRVRRRVNAANKKPEDLTLNDLETDVCTMDKCAGIFNVVSNSLGLVILLVLALFN

SEQ ID NO: 18 - protein, 368 aa, Plasmodium vivax
  misc_feature: CSP
MKNFILLAVSSILLVDLFPTHCGHNVDLSKAINLNGVNFNNVDASSLGAAHVGQSASRGRGLGENPDDEEGDAKKKKDGKKAEPKNPRENKLKQPGDRADGQPAGDRADGQPAGDRADGQPAGDRAGQPAGDRADGQPAGDRADGQPAGDRADGQPAGDRADGQPAGDRAGQPAGDRAGQPAGDRADGQPAGDRAGQPAGDRADGQPAGDRAGQPAGDRADGQPAGDRAGQPAGDRAGQPAGDRAGQPAGDRAGQPAGNGAGGQAAGGNAGGQGQNNEGANAPNEKSVKEYLDKVRATVGTEWTPCSVTCGVGVRVRRRVNAANKKPEDLTLNDLETDVCTMDKCAGIFNVVSNSLGLVILLVLALFN

SEQ ID NO: 19 - DNA, 1842 bp, Plasmodium berghei
  misc_feature: thrombospondin-related anonymous protein (TRAP)
ggatccgccaccatggctaagcttctggggaacagtaaatacttctttgtggtcctgctgctgtgcattagcgttttcctcaacggtcaggagattctggacgaaatcaagtactctgaggaggtctgcaacgaacaaatcgatctccacattctgctggatgggagtggcagcataggtcactctaactggatcagtcacgtgatacccatgctgacaacccttgtggacaatttgaacatcagccgcgatgagatcaatatctccatgaccttgttctccacttatgccagggaacttgtgagacttaagagatatgggtctacaagcaaagccagtctgaggttcatcatcgcgcaactccagaataactattctcctcatggaacgacaaatctgactagcgccctgttgaatgtggacaatctcattcagaagaaaatgaatcgccctaatgccattcagctcgtgattatccttactgacggcatccctaacaatctgaagaagtccactactgttgtcaaccagctgaagaagaaggacgtcaatgtcgctattattggtgttggcgccggagtaaacaatatgtttaaccgtatattggtaggatgtggaaaacttgggccttgtccctactactcttatggctcttgggatcaagcacaaaccatgatcaaaccatttctctcaaaggtctgtcaggaagtggagaaagtggcactgtgcggtaagtgggaggagtggagtgagtgttcaaccacttgcgacaacggaacgaaaataaggaagcgaaaggttctccatcccaactgtgccggggaaatgacagccccatgtaaagtgcgggactgtcctcccaaacctgtagcccctccggtcattcccatcaaagtccctgacgtgcctgtgaaaccagtcgaacctattgagcccgccgagccagcagagccagcagaaccagcagagcctgcagaacccgccgaacccgctgagcccgcggagcccgccgaacccgctgaaccggcagaacccgcggaaccagcggagcctgcagagccagctgagcctgctgaaccggcggagcccgctgaaccagccgagcctgctaaaccggccgaaccggcagagcccgctgagcctgccgagccagcggaaccagttaaccccgataatcctatcctgccgatcaagcccgaggagccatctggtggagccgagccattgaatccagaggtcgagaatccctttatcatccccgacgaacccatcgaacccattattgcgccaggagctgtaccggataagcccatcattcctgaggaatcaaatgagctgccaaacaatcttccagagtctccctccgatagtcaggtggagtatcctcggccaaacgacaatggggataacagcaacaacacaatcaattccaacaagaacataccaaataagcatgtgcctcctacagacgacaacccctacaagggccaggaagaacgaatccctaagccgcatcggagcaacgacgaatacatttactacaataatgctaacaataacgacaagctggagcccgagataccctctaaggattacgaggaaaacaagagcaagaaacagagcaaaagcaacaatggctataagatcgccggcggcataattggcgggctggctattatcggctgcattggagtgggctataacttcatagccgggtcctccgccgccgctatggctggagaggcggcaccttttgaggacgtgatggctgatgatgagaaggggatcgtggaaaacgaacagttcaaactgccagaggacaatgattggaattgactcgag

SEQ ID NO: 20 - protein, 607 aa, Plasmodium berghei
  misc_feature: TRAP
MAKLLGNSKYFFVVLLLCISVFLNGQEILDEIKYSEEVCNEQIDLHILLDGSGSIGHSNWISHVIPMLTTLVDNLNISRDEINISMTLFSTYARELVRLKRYGSTSKASLRFIIAQLQNNYSPHGTTNLTSALLNVDNLIQKKMNRPNAIQLVIILTDGIPNNLKKSTTVVNQLKKKDVNVAIIGVGAGVNNMFNRILVGCGKLGPCPYYSYGSWDQAQTMIKPFLSKVCQEVEKVALCGKWEEWSECSTTCDNGTKIRKRKVLHPNCAGEMTAPCKVRDCPPKPVAPPVIPIKVPDVPVKPVEPIEPAEPAEPAEPAEPAEPAEPAEPAEPAEPAEPAEPAEPAEPAEPAEPAEPAEPAEPAEPAKPAEPAEPAEPAEPAEPVNPDNPILPIKPEEPSGGAEPLNPEVENPFIIPDEPIEPIIAPGAVPDKPIIPEESNELPNNLPESPSDSQVEYPRPNDNGDNSNNTINSNKNIPNKHVPPTDDNPYKGQEERIPKPHRSNDEYIYYNNANNNDKLEPEIPSKDYEENKSKKQSKSNNGYKIAGGIIGGLAIIGCIGVGYNFIAGSSAAAMAGEAAPFEDVMADDEKGIVENEQFKLPEDNDWN

SEQ ID NO: 21 - protein, 606 aa, Plasmodium berghei
  misc_feature: TRAP
MKLLGNSKYFFVVLLLCISVFLNGQEILDEIKYSEEVCNEQIDLHILLDGSGSIGHSNWISHVIPMLTTLVDNLNISRDEINISMTLFSTYARELVRLKRYGSTSKASLRFIIAQLQNNYSPHGTTNLTSALLNVDNLIQKKMNRPNAIQLVIILTDGIPNNLKKSTTVVNQLKKKDVNVAIIGVGAGVNNMFNRILVGCGKLGPCPYYSYGSWDQAQTMIKPFLSKVCQEVEKVALCGKWEEWSECSTTCDNGTKIRKRKVLHPNCAGEMTAPCKVRDCPPKPVAPPVIPIKVPDVPVKPVEPIEPAEPAEPAEPAEPAEPAEPAEPAEPAEPAEPAEPAEPAEPAEPAEPAEPAEPAEPAEPAKPAEPAEPAEPAEPAEPVNPDNPILPIKPEEPSGGAEPLNPEVENPFIIPDEPIEPIIAPGAVPDKPIIPEESNELPNNLPESPSDSQVEYPRPNDNGDNSNNTINSNKNIPNKHVPPTDDNPYKGQEERIPKPHRSNDEYIYYNNANNNDKLEPEIPSKDYEENKSKKQSKSNNGYKIAGGIIGGLAIIGCIGVGYNFIAGSSAAAMAGEAAPFEDVMADDEKGIVENEQFKLPEDNDWN

SEQ ID NO: 22 - DNA, 1734 bp, Artificial Sequence
  thrombospondin-related anonymous protein (TRAP)
gccaccatggctaatcacttgggcaacgtgaagtacctggtgatagtgttcctgatcttcttcgacctgtttctggtcaacggcagagatgtgcagaacaacatagtcgacgagattaagtacagagaggaggtgtgcaatgacgaggtggatctctacctgctgatggactgtagcgggtcaatacgacggcacaattgggttaatcatgctgtccccttggctatgaagctgatccagcagctcaatctgaacgataatgccattcacctgtatgcttccgtgttcagcaataatgcaagggagatcattcgcctccacagtgacgctagtaagaataaggagaaagccctgatcatcatcaaatccctgcttagcaccaaccttccctatggcaagaccaatctcacagacgcgcttctgcaggtaaggaagcatctgaacgatcgcatcaacagagagaatgcgaatcagctggtagtaatcctgactgatgggattcccgattccatccaggacagcctgaaggaatcaaggaagctcagcgatagaggggtgaaaatcgcagttttcggaatcggtcaggggattaacgtggcctttaatcgctttctggtgggatgtcatccatctgatgggaaatgcaatctgtatgccgattctgcgtgggagaatgtgaagaacgtgattggcccctttatgaaagccgtgtgcgtagaggtggagaaaaccgcctcctgtggagtttgggacgaatggagcccctgttccgttacttgcgggaaagggacccgaagtcgcaagagagaaatccttcacgagggttgcacttctgaactccaagagcaatgcgaggaggaaaggtgtctccctaaacgagaaccccttgatgtgcctgacgaacctgaggacgatcaaccaagaccaaggggagacaacttcgccgtcgagaaacccaatgagaatatcattgacaacaatccacaggagccatcacctaatccagaggaaggcaaaggcgaaaaccctaacggttttgacttggacgagaatccggaaaatccacccaacccgcctaaccctccaaatccaccaaaccctccgaatcctcctaaccctgatatccccgagcaagagcctaatattccagaggatagcgaaaaggaggtcccctctgacgtccctaagaaccctgaagatgatcgcgaagagaactttgacatacccaagaaacccgagaataaacacgataatcagaacaatctcccgaacgataaatctgaccgctacattccatacagtccattgagcccaaaggtcttggataatgaacggaaacagtccgaccctcaatcacaggacaataacggtaatcgtcatgttccgaactccgaagatcgggaaacaaggccccatgggcgtaacaacgaaaaccggagctataaccggaagcacaacaacacacccaagcacccggaacgggaagaacatgagaagcccgataacaacaagaagaaggcaggctctgacaacaaatacaaaatagccggaggtattgccggaggattggcactgctggcctgcgctggccttgcttacaagttcgtcgttccaggcgccgcaacgccctatgcaggcgaaccagctccctttgacgagacactgggagaagaggacaaagacctggacgagcctgaacagttcagactgcccgaggaaaacgagtggaattaa

SEQ ID NO: 23 - protein, 575 aa, Artificial Sequence
  PfTRAP
MANHLGNVKYLVIVFLIFFDLFLVNGRDVQNNIVDEIKYREEVCNDEVDLYLLMDCSGSIRRHNWVNHAVPLAMKLIQQLNLNDNAIHLYASVFSNNAREIIRLHSDASKNKEKALIIIKSLLSTNLPYGKTNLTDALLQVRKHLNDRINRENANQLVVILTDGIPDSIQDSLKESRKLSDRGVKIAVFGIGQGINVAFNRFLVGCHPSDGKCNLYADSAWENVKNVIGPFMKAVCVEVEKTASCGVWDEWSPCSVTCGKGTRSRKREILHEGCTSELQEQCEEERCLPKREPLDVPDEPEDDQPRPRGDNFAVEKPNENIIDNNPQEPSPNPEEGKGENPNGFDLDENPENPPNPPNPPNPPNPPNPPNPDIPEQEPNIPEDSEKEVPSDVPKNPEDDREENFDIPKKPENKHDNQNNLPNDKSDRYIPYSPLSPKVLDNERKQSDPQSQDNNGNRHVPNSEDRETRPHGRNNENRSYNRKHNNTPKHPEREEHEKPDNNKKKAGSDNKYKIAGGIAGGLALLACAGLAYKFVVPGAATPYAGEPAPFDETLGEEDKDLDEPEQFRLPEENEWN

SEQ ID NO: 24 - protein, 560 aa, Artificial Sequence
  TRAP
MNHLGNVKYLVIVFLIFFDLFLVNGRDVQNNIVDEIKYREEVCNDEVDLYLLMDCSGSIRRHNWVNHAVPLAMKLIQQLNLNENAIHLYANVFSNNAREIIRLHSDASKNKEKALSIIKSLLSTNLPFGRTNLTDALLQVRKHLNDRINRENANQLVVILTDGIPNSIQDSLKESRKLSDRGVKIAVFGIGQGINVAFNRFLVGCHPSDGKCNLYADSAWENVKNVIGPFMKAVCVEVEKTASCGVWDEWSPCSVTCGKGTRSRKREILHEGCTSELQEQCEEERCPPKWEPLDVPDDPEDDQPRPRGDNSSVQKPEENIIDNNPQEPSPNPEEGKGENPNGFDLDENPENPPNPDIPQQEPNIPEDSEKEVPSDVPKNPEDDREENFDIPKKPENKHDNQNNLPNDKSDRYIPYSPLPPNVLDNERKQSDPQSQDNNGNRHVPNSEDRETRPHGRNNENRSYNRKHNDTPKHPEREEHEKPDNNKKKGGSSDNKYKIAGGIAGGLALLACAGLAYKFVVPGAATPYAGEPAPFDETLGEEDKDLDEPEQFRLPEENEWN

SEQ ID NO: 25 - DNA, 1680 bp, Artificial Sequence
  PvTRAPhumanCO
gccaccatggctaagctgctgcagaacaagtcttatctcctggtcgtgttcctgctttacgtatccatatttgcacggggagatgaaaaagttgtagatgaggtaaagtacagcgaggaggtatgcaacgaatctgtcgatctctatcttctcgtagatggctctggtagtattgggtatccgaactggataactaaggtaattcctatgctgaacggtcttattaatagtttgagcctcagtcgagacacgatcaatctctatatgaatctcttcggaaattacactacagaactcatacggctgggctctgggcagagtatagataaacgacaggctctgtcaaaggtgactgaacttagaaagacctacacgccatatggcactactaacatgacagccgcgcttgatgaagtccaaaagcaccttaatgatagagtgaaccgagagaaagcgatacaacttgtcatattgatgaccgacggggtgcccaactcaaaatacagggcgctggaggtcgcaaataaactgaagcaacgcaatgtttccttggccgttattggtgtcggacaaggaatcaaccatcagttcaatcggctgatcgcgggctgccgacctcgggagccgaactgcaaattttactcttatgctgattggaatgaggccgtagcacttataaagccctttattgctaaggtatgcacagaagttgagcgcgttgccaactgtggaccctgggacccctggaccgcgtgtagcgttacgtgcggcaggggaacgcactccaggtctcgcccttcattgcatgaaaagtgcactacccatatggtgtctgaatgtgaagagggggaatgcccagtggagcctgagccactgccagtacctgcgccactcccaacggttccagaagatgtaaatccgcgagacacggacgacgaaaatgagaacccgaacttcaacaagggactggacgtgccggatgaagatgatgacgaagtaccgcccgcgaatgaaggtgcggatggcaacccagtcgaggaaaatgttttcccgcctgctgacgatagcgttcctgatgagagtaacgtgctgcctctccctccggcagtcccaggcgggtcctctgaggagttcccggcggacgttcagaacaatcccgactctcctgaagagctgccgatggagcaagaagtgcctcaagataataacgtgaatgaaccagagagatccgatagcaacggttatggcgtaaatgagaaggtgatacctaatcccctcgacaatgagcgagacatggccaataagaataagaccgttcacccaggccggaaggacagcgcgagagatcgatatgcccgcccccatggttccactcatgtcaataataatagagccaatgagaactcagacataccaaacaacccagtaccttctgattacgaacagccggaggacaaggctaaaaagtcctccaataacggctataaaatcgctggtggagtgatcgctgggctcgcgttggttgggtgcgtcggtttcgcgtataacttcgtagcgggtggaggcgctgctggtatggcaggcgaaccggctccctttgacgaagctatggccgaagacgaaaaagacgtagccgaagcggaccagttcaaattgcctgaggacaacgattggaattaa

SEQ ID NO: 26 - protein, 557 aa, Plasmodium vivax
  misc_feature: PvTRAP
MAKLLQNKSYLLVVFLLYVSIFARGDEKVVDEVKYSEEVCNESVDLYLLVDGSGSIGYPNWITKVIPMLNGLINSLSLSRDTINLYMNLFGNYTTELIRLGSGQSIDKRQALSKVTELRKTYTPYGTTNMTAALDEVQKHLNDRVNREKAIQLVILMTDGVPNSKYRALEVANKLKQRNVSLAVIGVGQGINHQFNRLIAGCRPREPNCKFYSYADWNEAVALIKPFIAKVCTEVERVANCGPWDPWTACSVTCGRGTHSRSRPSLHEKCTTHMVSECEEGECPVEPEPLPVPAPLPTVPEDVNPRDTDDENENPNFNKGLDVPDEDDDEVPPANEGADGNPVEENVFPPADDSVPDESNVLPLPPAVPGGSSEEFPADVQNNPDSPEELPMEQEVPQDNNVNEPERSDSNGYGVNEKVIPNPLDNERDMANKNKTVHPGRKDSARDRYARPHGSTHVNNNRANENSDIPNNPVPSDYEQPEDKAKKSSNNGYKIAGGVIAGLALVGCVGFAYNFVAGGGAAGMAGEPAPFDEAMAEDEKDVAEADQFKLPEDNDWN

SEQ ID NO: 27 - protein, 556 aa, Plasmodium vivax
  misc_feature: TRAP
MKLLQNKSYLLVVFLLYVSIFARGDEKVVDEVKYSEEVCNESVDLYLLVDGSGSIGYPNWITKVIPMLNGLINSLSLSRDTINLYMNLFGNYTTELIRLGSGQSIDKRQALSKVTELRKSYSPYGTTNMTAALDEVQKHLNDRVNREKAIQLVILMTDGIPNSKYTALEVAKKLKQRNVSLAVIGIGQGINHQFNRLIAGCRPRESNCKFYSYADWNEAVALIKPFIAKVCTEVERVANCGPWDPWTACSVTCGRGTHSRSRPSLHEGCTTHMVSECEEGECPVEPEPLPVPAPLPTVPEDVNPRDTDDENENPNFNKGLDVPEEDDDEVPPANERADGNPVEENVFPPADDSVPDESNVLPLPPAVPGGSSEEFPADVQNNPDSPEELPMEQEVPQDNNVNEPERSDSNGYGVNEKVIPNPLDNERDMANKNKTVHPDRKDSARDRYARPHGSTHVNNNRANENSDIPNNPVPSDYEQPEDKAKKSSNNGYKIAGGVIAGLALVGCVGFAYNFVAGGGAAGMAGEPAPFDEAMAEDEKDVAEADQFKLPEDNDWN

SEQ ID NO: 28 - DNA, 1086 bp, Artificial Sequence
  inhibitor of cysteine proteases (ICP)
ggatccgccaccatggctaaatccattaccttctttgtgttcaacatttgcagcattttggctctgctgagtcactgtgaggacaatgacatctacagcttcgacattgtcaatgagacaaattggctgaagatcgccaagaacatcttcaaaggcaagtctcctagcaatttcacgatcataccgtttaacaataccggtagttctaacgataacgagtcaaacaaggaggaatcagtactgctgatcagaaagaagatcaaaagcaacaagaatcacgatagttccatcattagtggtgacactgttaacggggacattagtgacctgaattatacggctagcaacttttccgataactctgaggacatagaagataaccagaaatatcccacaaccagctacaatagtttcaaccatctcaattccaatatcgcctttaacgaagagtccgaatacattgagattaatagcgagtctgacttggagaacaagatcaaggacatcaacatcaaatccaatcttgaggaaaacaataccatgaacgaatccggcaaagtggatagcaagtatgagctcactggggacgagaaatgtggtaaaagcctgaagctcggcaacatcagcaatcagacaaaccaggaaaccataacccaaagcctgtcagttggagagattctgtgcattgacctcgaagggaatgcaggaacaggctatctgtgggtgttgctgggcatacacaaggatgagccaatcataaaccccgagaacttcccaaccaaactgacaaagaagtctttcttttccgaggaaataagtgtgactcagccaaagaagtacaagatcgatgagcatgattcttcaaagaatgtgaatcgcgaaatcgaaagccctgaacagaaggagtccgactcaaagcccaagaaacctcagatgcaacttcttggaggaccagatcggatgaggtcagtcatcaaaggacacaaacctggcaaatattacattgtgtactcttactatcgaccgttttctcccacttctggggcgaacactaaaatcatttacgtcacagtacagtgactcgag

SEQ ID NO: 29 - protein, 355 aa, Plasmodium berghei
  misc_feature: ICP
MAKSITFFVFNICSILALLSHCEDNDIYSFDIVNETNWLKIAKNIFKGKSPSNFTIIPFNNTGSSNDNESNKEESVLLIRKKIKSNKNHDSSIISGDTVNGDISDLNYTASNFSDNSEDIEDNQKYPTTSYNSFNHLNSNIAFNEESEYIEINSESDLENKIKDINIKSNLEENNTMNESGKVDSKYELTGDEKCGKSLKLGNISNQTNQETITQSLSVGEILCIDLEGNAGTGYLWVLLGIHKDEPIINPENFPTKLTKKSFFSEEISVTQPKKYKIDEHDSSKNVNREIESPEQKESDSKPKKPQMQLLGGPDRMRSVIKGHKPGKYYIVYSYYRPFSPTSGANTKIIYVTVQ

SEQ ID NO: 30 - protein, 354 aa, Plasmodium berghei
  misc_feature: ICP
MKSITFFVFNICSILALLSHCEDNDIYSFDIVNETNWLKIAKNIFKGKSPSNFTIIPFNNTGSSNDNESNKEESVLLIRKKIKSNKNHDSSIISGDTVNGDISDLNYTASNFSDNSEDIEDNQKYPTTSYNSFNHLNSNIAFNEESEYIEINSESDLENKIKDINIKSNLEENNTMNESGKVDSKYELTGDEKCGKSLKLGNISNQTNQETITQSLSVGEILCIDLEGNAGTGYLWVLLGIHKDEPIINPENFPTKLTKKSFFSEEISVTQPKKYKIDEHDSSKNVNREIESPEQKESDSKPKKPQMQLLGGPDRMRSVIKGHKPGKYYIVYSYYRPFSPTSGANTKIIYVTVQ

SEQ ID NO: 31 - DNA, 1251 bp, Artificial Sequence
  PfICPhumanCO
gccaccatggctaatctgttggtgttcttttgcttctttctgttgtcttgcatcgtacacctctctcgttgttctgacaacaattcatatagctttgagatcgtgaatcggagcacatggctcaacattgctgaacggatattcaaaggcaatgcaccattcaactttactattataccgtacaactatgtgaacaatagtactgaagagaataataacaaagactctgtccttctgataagcaagaatctgaagaattcctcaaacccagtggacgaaaataaccacatcattgactcaaccaagaagaacacgtccaataacaacaataataacagcaatatcgtcggaatatacgaaagtcaggtacatgaggagaagattaaagaggacaatacaagacaggataatatcaacaagaaggaaaacgagataatcaacaataaccatcagatccctgtgtccaacatcttttcagagaacattgacaacaacaagaactacattgagagcaactacaagagcacctataacaataatcccgagttgattcatagcacagatttcattggcagtaataacaaccacactttcaatttcctgtctcgctataacaactcagtgctgaacaacatgcaagggaataccaaagttccagggaatgttcccgaactgaaagcccgcattttctccgaggaagaaaacaccgaagtcgaaagcgccgaaaacaatcacactaacagtctgaatcctaacgagtcttgtgatcaaatcatcaaactgggcgatatcattaacagcgtcaatgagaagatcatcagcatcaatagtacggtgaataacgtgctctgcataaatctggattccgtcaatggcaatggcttcgtttggacccttcttggggtacacaagaagaaacccctgattgacccctccaattttcccactaaaagggtgactcagtcctacgtttcacctgacatttccgttacaaaccctgtgccaattccaaagaacagcaacaccaacaaagatgacagcatcaataataaacaggatggttcccagaataacacaacaacgaatcactttccgaagcctagagagcaactcgtgggtggatcttctatgctgatcagtaagatcaaaccccataaacccggaaagtatttcatcgtgtatagctactacagaccatttgaccctacaagggataccaacacccgaattgtggagctgaatgtccagtaa

SEQ ID NO: 32 - protein, 414 aa, Plasmodium falciparum
  misc_feature: PfICP
MANLLVFFCFFLLSCIVHLSRCSDNNSYSFEIVNRSTWLNIAERIFKGNAPFNFTIIPYNYVNNSTEENNNKDSVLLISKNLKNSSNPVDENNHIIDSTKKNTSNNNNNNSNIVGIYESQVHEEKIKEDNTRQDNINKKENEIINNNHQIPVSNIFSENIDNNKNYIESNYKSTYNNNPELIHSTDFIGSNNNHTFNFLSRYNNSVLNNMQGNTKVPGNVPELKARIFSEEENTEVESAENNHTNSLNPNESCDQIIKLGDIINSVNEKIISINSTVNNVLCINLDSVNGNGFVWTLLGVHKKKPLIDPSNFPTKRVTQSYVSPDISVTNPVPIPKNSNTNKDDSINNKQDGSQNNTTTNHFPKPREQLVGGSSMLISKIKPHKPGKYFIVYSYYRPFDPTRDTNTRIVELNVQ

SEQ ID NO: 33 - protein, 413 aa, Plasmodium falciparum
  misc_feature: ICP
MNLLVFFCFFLLSCIVHLSRCSDNNSYSFEIVNRSTWLNIAERIFKGNAPFNFTIIPYNYVNNSTEENNNKDSVLLISKNLKNSSNPVDENNHIIDSTKKNTSNNNNNNSNIVGIYESQVHEEKIKEDNTRQDNINKKENEIINNNHQIPVSNIFSENIDNNKNYIESNYKSTYNNNPELIHSTDFIGSNNNHTFNFLSRYNNSVLNNMQGNTKVPGNVPELKARIFSEEENTEVESAENNHTNSLNPNESCDQIIKLGDIINSVNEKIISINSTVNNVLCINLDSVNGNGFVWTLLGVHKKKPLIDPSNFPTKRVTQSYVSPDISVTNPVPIPKNSNTNKDDSINNKQDGSQNNTTTNHFPKPREQLVGGSSMLISKIKPHKPGKYFIVYSYYRPFDPTRDTNTRIVELNVQ

SEQ ID NO: 34 - DNA, 1104 bp, Artificial Sequence
  PvICPhumanCO
gccaccatggctaagttgtctagcctgttctgcctggttgtgtgttctagtgttgcccacctctcttcctgtagtgatcaaaacacttatagttttgatattgttaatcgaaacacttggtacagcatagccaagaaaatctttcaaggcacgaccccctgtaatttcactgtaatccctagttcctatgtcaacaactctgacggagtgtctacgagtgatgattccgtactgctcatccgcaaaaagctcaaggatccgagtgaagctggccttgacggatcttcagtttctggttcatccagttctggaaacagtcactccggttctgcaccttgttgtgataagggtacccccgctaaagaggcagagctgaaattttctacaaagtttgagggcgatgactatgctaagctgagagattctctgagccttatagacaagtcactccgagaagagtcaagctcagaggaggacagtaagatggaagatagtcaggtcggtgaagtaactcatgaagagactatcacctacaacatgcccgaagaatatatgccccagaacatttccgaggtattgatcggtgccgctgaagaggataggacatacgcgttgaagggggacgagccgtgtgatgtgtacttgaaacttggcgagataatcaatggaactaatgaaaagactatcgagtattctctccaaaaaaataagatactgtgcgttcaactcgaagcaattgggggaaatggatacctctgggctctcctcggcgtacacaaagaaaaaccccaaatcaacccagaggagtttccacgaaaaaagatcacaaaatcttttttcaccaatgagatatccgtcacgcagcctaaagcagtgcaaaagaacaaatctaataatggaggtgagagcagttcaaactcacctggctatgggaaaccccccgcaagcgaacagctcgggggatttgtgggtggcacatccatgcttcagagtatagtaaaggctcataaagagggcacctttttcgtagtttatagctactaccgccccttcgaccctaccgccaacgccaacacgaagatactcaaacttacggtttcatag

SEQ ID NO: 35 - protein, 365 aa, Artificial Sequence
  PVICP humanco + kozac
  misc_feature: PvICP
MAKLSSLFCLVVCSSVAHLSSCSDQNTYSFDIVNRNTWYSIAKKIFQGTTPCNFTVIPSSYVNNSDGVSTSDDSVLLIRKKLKDPSEAGLDGSSVSGSSSSGNSHSGSAPCCDKGTPAKEAELKFSTKFEGDDYAKLRDSLSLIDKSLREESSSEEDSKMEDSQVGEVTHEETITYNMPEEYMPQNISEVLIGAAEEDRTYALKGDEPCDVYLKLGEIINGTNEKTIEYSLQKNKILCVQLEAIGGNGYLWALLGVHKEKPQINPEEFPRKKITKSFFTNEISVTQPKAVQKNKSNNGGESSSNSPGYGKPPASEQLGGFVGGTSMLQSIVKAHKEGTFFVVYSYYRPFDPTANANTKILKLTVS

SEQ ID NO: 36 - protein, 359 aa, Plasmodium vivax
  misc_feature: ICP
MKLSSLFCLVVCSSVAHLSSCSDQNTYSFDIVNRNTWYSIAKKIFQGTTPCNFTVIPSSYVNNSDGVSTSDDSVLLIRKKLKDPSEAGLDGSSVSGNSHSGSAPCCDKGTPAKEAELNFSTKFEGDDYAKLRDSLSLIDKSLREESSSEEDSKMEDSQVGEVTHEETITYNMPEEYMPQNISEVLIGAAEEDRTYALKGDEPCDVYLKLGEIINGTNEKTIEYSLQKNKILCVQLEAIGGNGYLWALLGVHKEKPQINPEEFPRKKITKSFFTNEISVTQPKAVQKNKSNNGGESSSNSPGYGKPPASEQLGGFVGGTSMLQSIVKAHKEGTFFVVYSYYRPFDPTANANTKILKLTVS

SEQ ID NO: 37 - DNA, 2355 bp, Artificial Sequence
  Falcilysin, Bergheilysin-A-CO
ggatccgccaccatggctaaattgatgaaggttttgggttacatcaacattattaccaactgcgtgaatggaatcctctgcaaaggtgacaagaaaaggtactccatcttcaccaataattacatctacagcatatcaaccctcaataattatagctttgctgcaacaatgaacaaaatgcccgcttgggttaacgagaaatgtccagaacacaaaagctacgacatcgttgagaagcgctacaatgagaacctcaatctcacgtacacagtctatgagcacaagaaggccaagactcaggtcatagcactggggtctaacgatcctctcgacgctgagcaagcatttggcttctacgtgaaaaccctgacgcattcagataaaggcattccgcacatactggaacacactgtcctgagtggctctaagaatttcaactacaaggactcaatggggcttttggagaaaggcaccctgaacacacacctgaatgcctacaccttcaatgacaggaccatctacatggccgggagtatgaacaatagggatttctttaacattatggccgtctacatggatagcgtgttccagcctaacgtactggaaaacaaattcatcttccagacagagggatggacctatgaggtagagaagctgaaggaggaggagaagaacctcgacattcccaagattaaggactacaaggtgtcttttaacggaatcgtgtataatgagatgaagggtgcgtttagcaatcctctgcaggacctgtattatgaagtgatgagaaacatgttccccgacaacgtacacagtaacatctccggaggagatcctaaagaaataccaaatctgtcatatgaagagtttaaggagttttactacaagaattacaatccgaagaaaatcaaagtgttctttttctccaaaaataatccgacagagctgctcaactttgtggacaactatctctgtcagctggacttcacgaaatatcgggatgatgctgtggaacatgtcaattaccaagaataccgcaaaggcccattctatattaagaagaaattcgctgatcactcagaagagaaagaaaatcttgcctccgtgagttggcttctgaatcccaagaaacacaagaattccgatactgatctctctctggagtctcctacagactatttcgccttgctcatcatcaataatctgcttactcataccagtgagagcgtcctgtacaaagccctgatagaatccggattggggaatagtattgtagatcgagggctgaatgattccctggttcagtatgtgttcagcatcggcctgaaaggcataaaagagaagaacgagaagaacatctccctggacaaggtccactacgaggtggaaaagatcgttcttgaggcactgaaaaaagtggtcaaagaaggtttcaataagtcagcagttgaggcagccattaacaatattgagttcgtcctgaaagaagccaatctcaagatctccaaatctatagactttgtgtttgaaatggccagcagactgaactatggcaaagatccactgctgatctttgagtttgaaaagcatctcaacgtagtgaaggacaagatcaagaacgagcctaaatacctggagaagtatgtggagaaacatcttctgaacaatgatcatcgagtcgttattctgctggaaggggatgaaaactatggcaccgaacaggagaaactggaaaaggacatgctgaagaagcggattgaaagcttcactgagaaagaaaaggagaatattatcacagacttcgaaaatcttacgaagtacaagaacactgaggaatctcccgaacatctggacaagtttcccatcattagcattagcgacctcaatggaaagactttggagatccccgtgaaccctttctttaccaacctgaacaacgagaacaacatgcagcactataatgagacaaagaacaaccaaaccctggtcaaagaaaacatggaccgtttcattaacaaatacattctcaacaaggatggaaacgataagaacgacagcaagaacgcggatgtgccaatgctgatttatgaaatcccaacatctggcatattgtatctgcagtttatcttctctctggataaccttacactggaagaattgtcctacctgaatctgtttaaaagcttgatactggagaacaagactaacaagagatcaagtgaagagtttgtgatcttgcgggagaagaatattgggaatatgatgaccaatgttgctcttcttagcacatccgatcgcctcaatgtgactgacaaatataatgcgaaaggtttctttaactttgagtaactcgag

SEQ ID NO: 38 - protein, 778 aa, Plasmodium berghei
  misc_feature: Falcilysin, Bergheilysin-A
MAKLMKVLGYINIITNCVNGILCKGDKKRYSIFTNNYIYSISTLNNYSFAATMNKMPAWVNEKCPEHKSYDIVEKRYNENLNLTYTVYEHKKAKTQVIALGSNDPLDAEQAFGFYVKTLTHSDKGIPHILEHTVLSGSKNFNYKDSMGLLEKGTLNTHLNAYTFNDRTIYMAGSMNNRDFFNIMAVYMDSVFQPNVLENKFIFQTEGWTYEVEKLKEEEKNLDIPKIKDYKVSFNGIVYNEMKGAFSNPLQDLYYEVMRNMFPDNVHSNISGGDPKEIPNLSYEEFKEFYYKNYNPKKIKVFFFSKNNPTELLNFVDNYLCQLDFTKYRDDAVEHVNYQEYRKGPFYIKKKFADHSEEKENLASVSWLLNPKKHKNSDTDLSLESPTDYFALLIINNLLTHTSESVLYKALIESGLGNSIVDRGLNDSLVQYVFSIGLKGIKEKNEKNISLDKVHYEVEKIVLEALKKVVKEGFNKSAVEAAINNIEFVLKEANLKISKSIDFVFEMASRLNYGKDPLLIFEFEKHLNVVKDKIKNEPKYLEKYVEKHLLNNDHRVVILLEGDENYGTEQEKLEKDMLKKRIESFTEKEKENIITDFENLTKYKNTEESPEHLDKFPIISISDLNGKTLEIPVNPFFTNLNNENNMQHYNETKNNQTLVKENMDRFINKYILNKDGNDKNDSKNADVPMLIYEIPTSGILYLQFIFSLDNLTLEELSYLNLFKSLILENKTNKRSSEEFVILREKNIGNMMTNVALLSTSDRLNVTDKYNAKGFFNFE

SEQ ID NO: 39 - protein, 1149 aa, Plasmodium berghei
  misc_feature: Falcilysin, Bergheilysin-A
MKLMKVLGYINIITNCVNGILCKGDKKRYSIFTNNYIYSISTLNNYSFAATMNKMPAWVNEKCPEHKSYDIVEKRYNENLNLTYTVYEHKKAKTQVIALGSNDPLDAEQAFGFYVKTLTHSDKGIPHILEHTVLSGSKNFNYKDSMGLLEKGTLNTHLNAYTFNDRTIYMAGSMNNRDFFNIMAVYMDSVFQPNVLENKFIFQTEGWTYEVEKLKEEEKNLDIPKIKDYKVSFNGIVYNEMKGAFSNPLQDLYYEVMRNMFPDNVHSNISGGDPKEIPNLSYEEFKEFYYKNYNPKKIKVFFFSKNNPTELLNFVDNYLCQLDFTKYRDDAVEHVNYQEYRKGPFYIKKKFADHSEEKENLASVSWLLNPKKHKNSDTDLSLESPTDYFALLIINNLLTHTSESVLYKALIESGLGNSIVDRGLNDSLVQYVFSIGLKGIKEKNEKNISLDKVHYEVEKIVLEALKKVVKEGFNKSAVEAAINNIEFVLKEANLKISKSIDFVFEMASRLNYGKDPLLIFEFEKHLNVVKDKIKNEPKYLEKYVEKHLLNNDHRVVILLEGDENYGTEQEKLEKDMLKKRIESFTEKEKENIITDFENLTKYKNTEESPEHLDKFPIISISDLNGKTLEIPVNPFFTNLNNENNMQHYNETKNNQTLVKENMDRFINKYILNKDGNDKNDSKNADVPMLIYEIPTSGILYLQFIFSLDNLTLEELSYLNLFKSLILENKTNKRSSEEFVILREKNIGNMMTNVALLSTSDRLNVTDKYNAKGFFNFEMHMLSHKCNDALEIALEALKESDFSNKKKVIEILKRKINGMKTTFASKGHSILIKYVKSRINSKYYAYDLIHGYDNYLKLQEQLKLAETNYESLEAILNRIRKKIFKRNNLIMNVTVDPGTIDQLFAKSKNSFNNLLSYFDENESYCSKNDSFNKVVGWNKEIQEKKLLEGEEVKKELLVVPTFVNSVSMSGVLFNKGEYLDPSFTVIVAALKNSYLWETVRGLNGAYGVFADIEYDGTVVFLSARDPNLEKTLQTFREAAQGLRKMADVMTKNDLLRYIINAIGTIDRPRRGVELSKLSFSRIISNETEQDRIEFRNRVMNTKKEDFYKFADLLEKKVKEFEKNVVIITSKEKANEYINNVDNDFKKILIE

SEQ ID NO: 40 - DNA, 3591 bp, Artificial Sequence
  PfFalcilysin Human CO
gccaccatggctaatcttaccaaacttatgaaagtaataggatatataaacattataactaattgtgtacaatcttttactaacagagcggacaagaagaggtacaatgtttttgcaaagtctttcatcaatacgataaacacgaacctgtacaccttcaaagcggtgatgtcaaaaacgcctgaatggatccatgagaaatctcccaaacacaacagttatgacataattgagaaacgctataatgaagagttcaaaatgacatacacggtttatcaacataaaaaagcaaagacgcaggtaatttcactcggtacgaacgacccacttgatgtcgaacaggcctttgccttctatgtaaagactctgacccactctgggaaggggattccccacatcctcgaacacagtgtcctctcagggagtaaaaattacaactacaaaaattccattggactccttgagaagggaactctgcacacccaccttaacgcatacaccttcaatgaccgaactgtgtacatggccggttctatgaataacaaagacttcttcaatataatgggcgtctatatggacagtgtcttccaacctaatgtactggaaaacaaatacatattcgagacggagggatggacttacgaggtggaaaagctgaaagaggatgagaagggaaaagctgagattccacagatgaaagattataaagtatctttcaacggaattgtttataatgaaatgaagggtgccttgtcttccccgttggaagatctttaccatgaagagatgaagtatatgttcccagacaacgtccactctaataacagtggcggagacccaaaagagatcacaaacttgacctatgaggagttcaaggagttctattacaaaaactataatccaaaaaaagttaaagtgttcttcttttcaaagaacaacccaacggagcttctcaatttcgtagaccagtacctcggacagctggactacagcaaataccgcgacgatgctgttgaaagtgttgaataccaaacgtataaaaaaggacctttctacattaaaaaaaagtatggggaccatagcgaagagaaggagaatcttgtttccgtagcgtggcttctgaaccccaaggttgacaagactaacaatcataataataaccatagtaataaccaatctagcgaaaataatggttactccaacggctcccactctagcgatttgtccttggagaatcccacggactatttcgtgctcttgattatcaataacctccttatacataccccagaaagcgtcctgtacaaggccctcacagattgcgggctgggaaataatgtaattgataggggtttgaatgattcacttgtccaatacattttcagtattgggctgaagggaatcaaacgcaacaatgaaaaaattaaaaacttcgataaagtgcactatgaagtagaagacgtaattatgaatgctctcaaaaaagtggtcaaggagggattcaataaatccgccgtagaagccagcataaataatatcgagtttatcctgaaagaagccaatttgaaaacttcaaaatctatagatttcgtttttgagatgacttccaagctcaattataatagggatccactgctgatcttcgagtttgagaaatatcttaatattgtgaagaataagattaagaacgaacctatgtatttggagaagtttgttgaaaaacacttcatcaacaacgcacatcggtcagtgatccttcttgagggggacgagaactatgcacaggaacaggaaaaccttgagaaacaagaactgaagaaacgcatagagaacttcaatgagcaggagaaagagcaagtcattaagaacttcgaggagctgtccaagtacaagaacgcggaagagagcccggaacacttgaacaagtttccaataatctccatttccgacctcaataagaaaacactggaagtcccagttaacgtctacttcacgaacatcaacgaaaacaacaatataatggagacatataacaagctgaagacaaatgagcacatgcttaaggacaacatggacgtgtttctcaaaaaatacgttctcaaaaacgataagcacaacaccaacaataataacaacaataataataatatggactactctttcaccgaaactaaatatgaaggaaatgtgccaatccttgtgtacgaaatgccgacgactggaatagtctatttgcagttcgttttctccctcgatcacctgaccgtagacgagctcgcctatttgaatttgttcaagacacttatcttggagaacaagacaaacaaacgctccagtgaggatttcgtcattttgagagaaaaaaatattgggtcaatgtcagcgaatgtggcgctctacagcaaggacgaccacctgaacgtaaccgacaagtataacgcgcaagcactcttcaatctggaaatgcacgtactttctcataaatgcaacgatgcgctgaacattgcccttgaagctgttaaagaatctgacttcagtaataaaaaaaaggtcatagatatccttaagaggaagattaatggaatgaaaactacgttctcagaaaaaggatatgctatactgatgaaatacgttaaagcccatcttaatagcaagcattacgcccataacataatttatgggtacgaaaactatttgaagctgcaagagcaattggagcttgcagaaaacgattttaaaacattggagaatattttggtgagaataagaaacaaaatctttaacaaaaaaaacttgatggtcagcgtgacgtccgactatggtgctctcaaacacctgtttgtgaatagcaacgaatccttgaaaaaccttgtaagttacttcgaggaaaacgataagtatatcaacgatatgcagaataaagtgaatgaccctaccgtcatggggtggaatgaagaaatcaagtctaaaaagctgttcgacgaggagaaggtgaaaaaggagttctttgtcttgccgacctttgtcaacagcgtctcaatgagtggtatcctgttcaagccgggcgaatatctcgacccgagtttcactgtcatcgttgcggccctgaaaaatagctatttgtgggacacagttcgaggacttaacggggcttacggtgtattcgctgatatcgaatacgatggctctgtagtatttctttcagctcgagaccccaatctcgaaaagaccctcgccactttccgagaatctgctaaaggattgcgcaaaatggctgacaccatgacagagaacgatttgcttcggtatattattaacacaattggcacgatcgacaagcctcgacgagggatagaactttctaagctgtcattccttagacttatttccaacgagtcagagcaagatcgggtggagttccggaaacggataatgaacacaaaaaaggaagatttctataaattcgcagatcttcttgagagcaaagtaaatgaattcgaaaaaaatatcgttatcataacaacaaaagaaaaggccaacgaatatatagcaaacgtagatggcgagtttaagaaagtcttgatcgagtga

SEQ ID NO: 41 - protein, 1194 aa, Plasmodium falciparum
  misc_feature: PfFalcilysin
MANLTKLMKVIGYINIITNCVQSFTNRADKKRYNVFAKSFINTINTNLYTFKAVMSKTPEWIHEKSPKHNSYDIIEKRYNEEFKMTYTVYQHKKAKTQVISLGTNDPLDVEQAFAFYVKTLTHSGKGIPHILEHSVLSGSKNYNYKNSIGLLEKGTLHTHLNAYTFNDRTVYMAGSMNNKDFFNIMGVYMDSVFQPNVLENKYIFETEGWTYEVEKLKEDEKGKAEIPQMKDYKVSFNGIVYNEMKGALSSPLEDLYHEEMKYMFPDNVHSNNSGGDPKEITNLTYEEFKEFYYKNYNPKKVKVFFFSKNNPTELLNFVDQYLGQLDYSKYRDDAVESVEYQTYKKGPFYIKKKYGDHSEEKENLVSVAWLLNPKVDKTNNHNNNHSNNQSSENNGYSNGSHSSDLSLENPTDYFVLLIINNLLIHTPESVLYKALTDCGLGNNVIDRGLNDSLVQYIFSIGLKGIKRNNEKIKNFDKVHYEVEDVIMNALKKVVKEGFNKSAVEASINNIEFILKEANLKTSKSIDFVFEMTSKLNYNRDPLLIFEFEKYLNIVKNKIKNEPMYLEKFVEKHFINNAHRSVILLEGDENYAQEQENLEKQELKKRIENFNEQEKEQVIKNFEELSKYKNAEESPEHLNKFPIISISDLNKKTLEVPVNVYFTNINENNNIMETYNKLKTNEHMLKDNMDVFLKKYVLKNDKHNTNNNNNNNNNMDYSFTETKYEGNVPILVYEMPTTGIVYLQFVFSLDHLTVDELAYLNLFKTLILENKTNKRSSEDFVILREKNIGSMSANVALYSKDDHLNVTDKYNAQALFNLEMHVLSHKCNDALNIALEAVKESDFSNKKKVIDILKRKINGMKTTFSEKGYAILMKYVKAHLNSKHYAHNIIYGYENYLKLQEQLELAENDFKTLENILVRIRNKIFNKKNLMVSVTSDYGALKHLFVNSNESLKNLVSYFEENDKYINDMQNKVNDPTVMGWNEEIKSKKLFDEEKVKKEFFVLPTFVNSVSMSGILFKPGEYLDPSFTVIVAALKNSYLWDTVRGLNGAYGVFADIEYDGSVVFLSARDPNLEKTLATFRESAKGLRKMADTMTENDLLRYIINTIGTIDKPRRGIELSKLSFLRLISNESEQDRVEFRKRIMNTKKEDFYKFADLLESKVNEFEKNIVIITTKEKANEYIANVDGEFKKVLIE

SEQ ID NO: 42 - protein, 1193 aa, Plasmodium falciparum
  misc_feature: Falcilysin
MNLTKLMKVIGYINIITNCVQSFTNRADKKRYNVFAKSFINTINTNLYTFKAVMSKTPEWIHEKSPKHNSYDIIEKRYNEEFKMTYTVYQHKKAKTQVISLGTNDPLDVEQAFAFYVKTLTHSGKGIPHILEHSVLSGSKNYNYKNSIGLLEKGTLHTHLNAYTFNDRTVYMAGSMNNKDFFNIMGVYMDSVFQPNVLENKYIFETEGWTYEVEKLKEDEKGKAEIPQMKDYKVSFNGIVYNEMKGALSSPLEDLYHEEMKYMFPDNVHSNNSGGDPKEITNLTYEEFKEFYYKNYNPKKVKVFFFSKNNPTELLNFVDQYLGQLDYSKYRDDAVESVEYQTYKKGPFYIKKKYGDHSEEKENLVSVAWLLNPKVDKTNNHNNNHSNNQSSENNGYSNGSHSSDLSLENPTDYFVLLIINNLLIHTPESVLYKALTDCGLGNNVIDRGLNDSLVQYIFSIGLKGIKRNNEKIKNFDKVHYEVEDVIMNALKKVVKEGFNKSAVEASINNIEFILKEANLKTSKSIDFVFEMTSKLNYNRDPLLIFEFEKYLNIVKNKIKNEPMYLEKFVEKHFINNAHRSVILLEGDENYAQEQENLEKQELKKRIENFNEQEKEQVIKNFEELSKYKNAEESPEHLNKFPIISISDLNKKTLEVPVNVYFTNINENNNIMETYNKLKTNEHMLKDNMDVFLKKYVLKNDKHNTNNNNNNNNNMDYSFTETKYEGNVPILVYEMPTTGIVYLQFVFSLDHLTVDELAYLNLFKTLILENKTNKRSSEDFVILREKNIGSMSANVALYSKDDHLNVTDKYNAQALFNLEMHVLSHKCNDALNIALEAVKESDFSNKKKVIDILKRKINGMKTTFSEKGYAILMKYVKAHLNSKHYAHNIIYGYENYLKLQEQLELAENDFKTLENILVRIRNKIFNKKNLMVSVTSDYGALKHLFVNSNESLKNLVSYFEENDKYINDMQNKVNDPTVMGWNEEIKSKKLFDEEKVKKEFFVLPTFVNSVSMSGILFKPGEYLDPSFTVIVAALKNSYLWDTVRGLNGAYGVFADIEYDGSVVFLSARDPNLEKTLATFRESAKGLRKMADTMTENDLLRYIINTIGTIDKPRRGIELSKLSFLRLISNESEQDRVEFRKRIMNTKKEDFYKFADLLESKVNEFEKNIVIITTKEKANEYIANVDGEFKKVLIE

SEQ ID NO: 43 - DNA, 3471 bp, Artificial Sequence
  PvFalcilysin Human CO
gccaccatggctaaactgatgagggtctttggttatcttaacataattactaactgcgttaacggacttctctgtaaagcggagaaacgcaaatataacgtctttacgaacagctttatctattctatctctacaacaaatctgtactccttcacggccatgatgaacaaatcacccgagtgggtgcaggaaaagtgtcccgagcacaagtcttataacatccttgaaaagcggttttctgataaattccaaatgacgtatacagtgtacgagcacaaaaaagcgaaaacccaagtcattgccctcggcagcaatgaccctctggacgttgagcagacttttgccttttacgtaaagaccctgacgaactcaggtaagggtattcctcacatactcgagcacacagttttgagcgggagcaaaaatttcaattacaaagatagcatgggactcctggaaaagggaacacttaacacccatttgaatgcgtatactttcaacgacaggactgtgtatatggcggggtctatgaataatagagactttttcaacataatggcggtgtatatggatagcgtgtttcaaccgaatgttcttgaaaataagtttatcttccaaacggaaggatggacctacgaagtggaaaagttgaaagatgaggagaagaacgctgatgtaccgaaaattaaagattataaggtttcctacaatggtatagtctattccgaaatgaaagggagcttctcctctcccctgcaatatctttattatttgatcatgaaaaacatcttccctgacaacgtccattctaacataagtggaggggaccctaaggagattccaacgctcacgtatgaggaattcaaagagttctactataagaattacaatcctaaaaaaattaaagtcattttcttcagcaaaaacaatcctacagagttgttgaatttcgtcgacgattatttgaaccagctcgactttactaaatacagagacgatgcagtagaaaacgttaattatcaggagtacaagaaggggccattctatgttaagaagaaatttgcggatcacagtgaggaaaaggaaaatcttgtttccatttcatggcttctcaacccgaaaaaaaatgacctcctggatgttgatcttagccttgaaagcccccaagactacttcgctttgttgattattaacaaccttctgacgcacaccaccgaatctgtgctttacaaggcgcttattgactgcggtttgggtaacaccgttatcgatacaggccttgacgacagcctggtacagttcatattttctataggtctgaagggtatcaaggaaaagaatgaaaagaacgtatcattggacgttgtccattatgaagtggaaaaggtggttctcaaagcgctccaaaaagtagtggatgaaggcttcaacaaatcagcggtcgaggccagcattaacaacatagaattcgttttgaaagaagccaatcttaaaacgtccaaaagcgtggactacatatttgaaatggcttccaggctgaattataatagagaccctctgctgatctttgaattcgagaagcacttgaacgtcgttaaagataagatcaagaatgaaccaaagtatttggagaaattcattgagaaacactttataaacaataaccatcgggctgtcatcttgatggagggtgatgaaaactatgggaaagaacaggaggatttggaaaaggagactttgaaaaagaagatcgaatcactcacggaaaaagaacgggacgacataatagttgatttcgagaacctgacaaagtataagaacatggtcgagagccccgaacatttggataattttcccatcatctctatcagcgatctgaacaaagaaacgctggaaatccctgctaatgcatatttcacgtccacggcagaggaaaataatatggaaaagtacaataaagtgaaggcaagcgaggatgttatgaagaaaaacatggaccacctcatcgacaaatatgttctgaaaggagcgcaaggaggcgctgctaccgacggtgcagccaaacagggtgattccagcgatggtgaaatccccatgctcgtttacgagatgcctaccagtggaatcttgtatctgcaatttatctttaacctcgatcatctgagcctggaggaaatgtcatatctcaatctctttaagatgctcatccttgagaacaaaactatgaaacgctcctctgaggagtttgtcattttgagggaaaagaatatcggcaacataatggctaacgtcgccttgtatagtatcagcgatcacctcaaggtcacttctaaatacaacgctcacggtctgttcaattttgaaatgcatgtattgagtcacaagtgcaatgagtctttggaaattgccttggaagctctcaaggactctgattttagcaacaagaagaaaatagtggagatcctgaaacggaagatcaacggcatgaagacggtcttctcttctaaaggctactcacttctcctcaagtatgtaaagtcacaaatgaatgcaaaatactacgctcatgatttggtttttggatacggcaactatttgaagttgcaagaacagctcaagctcgccgaaagtgactttccacagttcgagcagattctcaacagaatccgaaataagattttcactaagaagaatctgctgatcagcgtgaccagtgacgctgcagcgttggatcaactgtttgtgcatagcaaggaatccctgaagaaccttcttgggtatttcgaagagaatgatgccaagtctggagaggctgagaccatagggtggaatgaggagattaaacaatcaaaagtgatcgaaaaggaacaaaagaagaaggaattctttgtaataccaacatttgtcaatgcagtatcaatggctggaatgttgtttaatgagaaggagttcctggatccgtctttcatagttatcgtggccgcattgaaaaactcatacctttgggagaccgtgaggggactcaacggggcatatggagtttttgctgatatagaatacgatggtgccgtcgtgttcttgtcagcccgcgatccgaatctggaaaagacgctgcaaacgttcaaggagtctgctcaagggttgagaaagatggcggatacaatgaccaaaaatgaccttcggcgctacataatcaatgcgatcggcaatattgataagccgagacggggtgttgagctctcaaagctctcacttttgagaattatatctaatgaaaccaaacaggaccgaatcgactttcgcaagaggattatggagacgacgaaggaagacttctataaattcgctgacttgcttgaaaagaagattgcagaatttgaaaagaatattgttattatcacatctaaggaaaaagcctcagaatacagcactaacgtagaccaggattttaagcagattcatatcgagtag

SEQ ID NO: 44 - protein, 1154 aa, Plasmodium vivax
  misc_feature: PvFalcilysin
MAKLMRVFGYLNIITNCVNGLLCKAEKRKYNVFTNSFIYSISTTNLYSFTAMMNKSPEWVQEKCPEHKSYNILEKRFSDKFQMTYTVYEHKKAKTQVIALGSNDPLDVEQTFAFYVKTLTNSGKGIPHILEHTVLSGSKNFNYKDSMGLLEKGTLNTHLNAYTFNDRTVYMAGSMNNRDFFNIMAVYMDSVFQPNVLENKFIFQTEGWTYEVEKLKDEEKNADVPKIKDYKVSYNGIVYSEMKGSFSSPLQYLYYLIMKNIFPDNVHSNISGGDPKEIPTLTYEEFKEFYYKNYNPKKIKVIFFSKNNPTELLNFVDDYLNQLDFTKYRDDAVENVNYQEYKKGPFYVKKKFADHSEEKENLVSISWLLNPKKNDLLDVDLSLESPQDYFALLIINNLLTHTTESVLYKALIDCGLGNTVIDTGLDDSLVQFIFSIGLKGIKEKNEKNVSLDVVHYEVEKVVLKALQKVVDEGFNKSAVEASINNIEFVLKEANLKTSKSVDYIFEMASRLNYNRDPLLIFEFEKHLNVVKDKIKNEPKYLEKFIEKHFINNNHRAVILMEGDENYGKEQEDLEKETLKKKIESLTEKERDDIIVDFENLTKYKNMVESPEHLDNFPIISISDLNKETLEIPANAYFTSTAEENNMEKYNKVKASEDVMKKNMDHLIDKYVLKGAQGGAATDGAAKQGDSSDGEIPMLVYEMPTSGILYLQFIFNLDHLSLEEMSYLNLFKMLILENKTMKRSSEEFVILREKNIGNIMANVALYSISDHLKVTSKYNAHGLFNFEMHVLSHKCNESLEIALEALKDSDFSNKKKIVEILKRKINGMKTVFSSKGYSLLLKYVKSQMNAKYYAHDLVFGYGNYLKLQEQLKLAESDFPQFEQILNRIRNKIFTKKNLLISVTSDAAALDQLFVHSKESLKNLLGYFEENDAKSGEAETIGWNEEIKQSKVIEKEQKKKEFFVIPTFVNAVSMAGMLFNEKEFLDPSFIVIVAALKNSYLWETVRGLNGAYGVFADIEYDGAVVFLSARDPNLEKTLQTFKESAQGLRKMADTMTKNDLRRYIINAIGNIDKPRRGVELSKLSLLRIISNETKQDRIDFRKRIMETTKEDFYKFADLLEKKIAEFEKNIVIITSKEKASEYSTNVDQDFKQIHIE

SEQ ID NO: 45 - protein, 1153 aa, Plasmodium vivax
  misc_feature: Falcilysin
MKLMRVFGYLNIITNCVNGLLCKAEKRKYNVFTNSFIYSISTTNLYSFTAMMNKSPEWVQEKCPEHKSYNILEKRFSDKFQMTYTVYEHKKAKTQVIALGSNDPLDVEQTFAFYVKTLTNSGKGIPHILEHTVLSGSKNFNYKDSMGLLEKGTLNTHLNAYTFNDRTVYMAGSMNNRDFFNIMAVYMDSVFQPNVLENKFIFQTEGWTYEVEKLKDEEKNADVPKIKDYKVSYNGIVYSEMKGSFSSPLQYLYYLIMKNIFPDNVHSNISGGDPKEIPTLTYEEFKEFYYKNYNPKKIKVIFFSKNNPTELLNFVDDYLNQLDFTKYRDDAVENVNYQEYKKGPFYVKKKFADHSEEKENLVSISWLLNPKKNDLLDVDLSLESPQDYFALLIINNLLTHTTESVLYKALIDCGLGNTVIDTGLDDSLVQFIFSIGLKGIKEKNEKNVSLDVVHYEVEKVVLKALQKVVDEGFNKSAVEASINNIEFVLKEANLKTSKSVDYIFEMASRLNYNRDPLLIFEFEKHLNVVKDKIKNEPKYLEKFIEKHFINNNHRAVILMEGDENYGKEQEDLEKETLKKKIESLTEKERDDIIVDFENLTKYKNMVESPEHLDNFPIISISDLNKETLEIPANAYFTSTAEENNMEKYNKVKASEDVMKKNMDHLIDKYVLKGAQGGAATDGAAKQGDSSDGEIPMLVYEMPTSGILYLQFIFNLDHLSLEEMSYLNLFKMLILENKTMKRSSEEFVILREKNIGNIMANVALYSISDHLKVTSKYNAHGLFNFEMHVLSHKCNESLEIALEALKDSDFSNKKKIVEILKRKINGMKTVFSSKGYSLLLKYVKSQMNAKYYAHDLVFGYGNYLKLQEQLKLAESDFPQFEQILNRIRNKIFTKKNLLISVTSDAAALDQLFVHSKESLKNLLGYFEENDAKSGEAETIGWNEEIKQSKVIEKEQKKKEFFVIPTFVNAVSMAGMLFNEKEFLDPSFIVIVAALKNSYLWETVRGLNGAYGVFADIEYDGAVVFLSARDPNLEKTLQTFKESAQGLRKMADTMTKNDLRRYIINAIGNIDKPRRGVELSKLSLLRIISNETKQDRIDFRKRIMETTKEDFYKFADLLEKKIAEFEKNIVIITSKEKASEYSTNVDQDFKQIHIE

SEQ ID NO: 46 - DNA, 1227 bp, Artificial Sequence
  Falcilysin, Bergheilysin-B-CO
ggatccgccaccatggctaaactcatgaaggtcttggggtatatcaacatcatcacgaattgcgtgaatggcatactgtgtaaaggcgataagaagcgatacatgcacatgctctcccataagtgtaacgacgctctggagattgcgctggaagccctgaaagaaagcgatttcagcaataagaagaaagtgattgaaatcctgaagaggaaaatcaatggcatgaaaaccacgtttgccagcaaaggtcactcaatactgattaagtacgtgaagtctcggattaatagcaaatattacgcttatgacctgattcatgggtatgacaactatctgaagctccaggaacagcttaaactggcggagacaaactacgagagtctggaagccatacttaatcgcatcaggaagaagatctttaagcggaacaatctgatcatgaacgtgactgtcgatccaggaactattgatcagctgtttgccaaatccaagaatagcttcaacaaccttctgtcatactttgacgagaacgagagctactgctctaagaatgactccttcaacaaagtggtaggctggaacaaggagattcaggagaagaagctgcttgaaggagaggaggtcaagaaagagctcttggtcgtacccacatttgtgaacagtgtctctatgagtggagtgctgttcaacaaaggcgaatatcttgacccctcattcaccgtaatcgttgcagccttgaaaaactcatacctttgggagacagttcgaggactgaatggagcttatggggtgtttgccgacatagagtatgacggtactgtcgttttcctgagtgcaagagatccgaatctggaaaagactctccaaacctttcgtgaagcagcacaaggtctgcgtaaaatggctgatgtgatgacaaagaatgatcttctgcggtatatcattaacgctattgggaccatcgatagacctcgcagaggcgttgagttgtccaagctgtccttctctcgcataataagcaacgaaaccgaacaggacaggattgaattcaggaaccgggtgatgaataccaagaaagaggatttctacaaatttgcggatttgctcgaaaagaaagtcaaggaatttgagaagaatgtggtgatcatcacatctaaggagaaagccaacgagtacatcaacaatgttgacaatgacttcaagaagatcctcattgagtaactcgag

SEQ ID NO: 47 - protein, 402 aa, Plasmodium Berghei
  misc_feature: Falcilysin, Bergheilysin-B
MAKLMKVLGYINIITNCVNGILCKGDKKRYMHMLSHKCNDALEIALEALKESDFSNKKKVIEILKRKINGMKTTFASKGHSILIKYVKSRINSKYYAYDLIHGYDNYLKLQEQLKLAETNYESLEAILNRIRKKIFKRNNLIMNVTVDPGTIDQLFAKSKNSFNNLLSYFDENESYCSKNDSFNKVVGWNKEIQEKKLLEGEEVKKELLVVPTFVNSVSMSGVLFNKGEYLDPSFTVIVAALKNSYLWETVRGLNGAYGVFADIEYDGTVVFLSARDPNLEKTLQTFREAAQGLRKMADVMTKNDLLRYIINAIGTIDRPRRGVELSKLSFSRIISNETEQDRIEFRNRVMNTKKEDFYKFADLLEKKVKEFEKNVVIITSKEKANEYINNVDNDFKKILIE

SEQ ID NO: 48 - DNA, 2454 bp, Plasmodium Berghei
  misc_feature: perforin like protein 1 (SPECT2)
ggatccgccaccatggctaagatgcggaacattaagaaatcacttccggtgctcttcattctcttgtgcatttatcagcagtactttataaactctctccgtatttccgtccgcaacaataagaaccacagggatgaaaacaaattcaacaagaatatggagcttggtacgatggagaaacccatcaacattctttgcaatgacgtcagctgtaacacagagaataacatttctttcgtaaaccagaagaaaaaggaaatagatagtgacagcgacctctataacatgttcgatgacgacgcctctacctccgctggtgatgacgaagatgactatgacgactacacagatgataagaacgctgagatcaaagatgaagagcaaaatgagcacatcgacaagatcgaccagaagaaggataagaaacgcacattctctatcaataagcaggaggaagaaatcaacgaaaataaaaacaaaacggagaaattcttcaagaaatacaagtttaacgacgccaactcagaaggggacgacgatgagtcagacaccgatgacgagaatttggacaactccacggagaacagctacgaggaaaacaagaatcccgagaacgttatcgacaagcatatggccgtatttcctgggctctattttgtggggatcggctacgatattctgttcgggaatcctctcggagaaaccgattctctgagcgaccctggttatagagcacagatttacctgttgaattgggaattctcaaaccatggcatcgccaacgatctgcacacgctccaaccaattaatgcctggattcggaaagagaatgcatgctcacgagttgaatccatcaacgaatgttcctctgttagtgagtatacaaagaatctgtcagtggacgtaagtgtatcaggcagctatatgggcttcggcagtttctccgctagcactgggtacaagaaattcattaacgagataagcaagagaacatccaagacctactttataaagagcaactgcatcaagtataccatcggacttccaccttatgtgccatgggagcataccactgcttacatgaatgcggtgaatattctgccaaaggaatttaccggcctggatggagacagcgaatgcacaccggatgtttacgagcagaagaagatgactaaacagtgtaagaacgtgcaactgtggattcagttctttaagacctatggtacacacataatcgtggaggctcaattgggagggaaaataaccaaaatcatcaatgtctctaacacaagcgtgaaccagatgaagaaggatggcgtcagtgtgaaagcccagatccaggctcagtttggttttgcaagtgtgggcggtagtacaagcgtgtcaagtgacaatagcactaagaacgacaatagcagctacgacatgtctgagaaactcgtggttatcggagggaatcctataaaagacgtcaccaaagaggagaatctgtacgaatggagcaaaacagtgtcctctaaccccatgcctatccacatcaagctgctgccaatctataagtccttcgatagtgaggaactgaaagagtcttacgagaaagcggttctctactacaccaggctttatggcagctctccccacggaactattcagaaagatgagaacgacatcatcaaaatcttgacggccagtaccaccatcactaaaattggtgctccacccataacagcggaatgtccacataatcaagtggtgctgtttgggtatgtcctgaagcagaacttctgggacaacacctccaacctgaagggatacgacattgagatctgtgaggctggactgaatagttgcacgtccaaacagggaagtacaaacaagtacgatgtgagctatctgtacattgaatgtggcacacaggcaatgtcattctccgatcaagtcataaccgcatccaacactacttacaataccatcaagtgtcccaatgactacactattatctttggattcgggttttcctctagctccggtaagggagtttctgccatgcacacccacattacatcctgtagacccggcatgaaaagctgctccctgaatatgggcaacagcaatgacaagaactacatgtacctggtgtgcgtcgatgccacaatctggtctggcattaatgagctgactattgtggccaaagatgattttcacggcgcagtgaataggtctaagcagttcaatgatggcgaattggtactgtcctgtcaggaaaatggcactatcctgacagggttcactggggagactcatacctctagcccgtatgtcaagagcccttttagcaagtgtcttaaatcactgaagagctgctcagtccatgggagtggacaatctatcggatataccaactataagtcactgttttccatcatactgtgcaagaacggtgagtgactcgag

SEQ ID NO: 49 - protein, 811 aa, Plasmodium Berghei
  misc_feature: perforin like protein 1 (SPECT2)
MAKMRNIKKSLPVLFILLCIYQQYFINSLRISVRNNKNHRDENKFNKNMELGTMEKPINILCNDVSCNTENNISFVNQKKKEIDSDSDLYNMFDDDASTSAGDDEDDYDDYTDDKNAEIKDEEQNEHIDKIDQKKDKKRTFSINKQEEEINENKNKTEKFFKKYKFNDANSEGDDDESDTDDENLDNSTENSYEENKNPENVIDKHMAVFPGLYFVGIGYDILFGNPLGETDSLSDPGYRAQIYLLNWEFSNHGIANDLHTLQPINAWIRKENACSRVESINECSSVSEYTKNLSVDVSVSGSYMGFGSFSASTGYKKFINEISKRTSKTYFIKSNCIKYTIGLPPYVPWEHTTAYMNAVNILPKEFTGLDGDSECTPDVYEQKKMTKQCKNVQLWIQFFKTYGTHIIVEAQLGGKITKIINVSNTSVNQMKKDGVSVKAQIQAQFGFASVGGSTSVSSDNSTKNDNSSYDMSEKLVVIGGNPIKDVTKEENLYEWSKTVSSNPMPIHIKLLPIYKSFDSEELKESYEKAVLYYTRLYGSSPHGTIQKDENDIIKILTASTTITKIGAPPITAECPHNQVVLFGYVLKQNFWDNTSNLKGYDIEICEAGLNSCTSKQGSTNKYDVSYLYIECGTQAMSFSDQVITASNTTYNTIKCPNDYTIIFGFGFSSSSGKGVSAMHTHITSCRPGMKSCSLNMGNSNDKNYMYLVCVDATIWSGINELTIVAKDDFHGAVNRSKQFNDGELVLSCQENGTILTGFTGETHTSSPYVKSPFSKCLKSLKSCSVHGSGQSIGYTNYKSLFSIILCKNGE

SEQ ID NO: 50 - protein, 810 aa, Plasmodium berghei
  misc_feature: SPECT2
MKMRNIKKSLPVLFILLCIYQQYFINSLRISVRNNKNHRDENKFNKNMELGTMEKPINILCNDVSCNTENNISFVNQKKKEIDSDSDLYNMFDDDASTSAGDDEDDYDDYTDDKNAEIKDEEQNEHIDKIDQKKDKKRTFSINKQEEEINENKNKTEKFFKKYKFNDANSEGDDDESDTDDENLDNSTENSYEENKNPENVIDKHMAVFPGLYFVGIGYDILFGNPLGETDSLSDPGYRAQIYLLNWEFSNHGIANDLHTLQPINAWIRKENACSRVESINECSSVSEYTKNLSVDVSVSGSYMGFGSFSASTGYKKFINEISKRTSKTYFIKSNCIKYTIGLPPYVPWEHTTAYMNAVNILPKEFTGLDGDSECTPDVYEQKKMTKQCKNVQLWIQFFKTYGTHIIVEAQLGGKITKIINVSNTSVNQMKKDGVSVKAQIQAQFGFASVGGSTSVSSDNSTKNDNSSYDMSEKLVVIGGNPIKDVTKEENLYEWSKTVSSNPMPIHIKLLPIYKSFDSEELKESYEKAVLYYTRLYGSSPHGTIQKDENDIIKILTASTTITKIGAPPITAECPHNQVVLFGYVLKQNFWDNTSNLKGYDIEICEAGLNSCTSKQGSTNKYDVSYLYIECGTQAMSFSDQVITASNTTYNTIKCPNDYTIIFGFGFSSSSGKGVSAMHTHITSCRPGMKSCSLNMGNSNDKNYMYLVCVDATIWSGINELTIVAKDDFHGAVNRSKQFNDGELVLSCQENGTILTGFTGETHTSSPYVKSPFSKCLKSLKSCSVHGSGQSIGYTNYKSLFSIILCKNGE

SEQ ID NO: 51 - DNA, 2538 bp, Artificial Sequence
  PfSPECT2 human CO
gccaccatggctaagctgagaatcctcaagaagcactattatgtcgtgtttatccttctgtatctgtatgacatcagctgcttcaagtgcattcggctgaacaaccgttccatctacaagaacaagtacaagaataacgtgcacataggaaccaatgagaatatcaggagtattgagaagtatagcaacgtgctgtgtaacagcatcttgtgtaagaacgataaaatctctagcttcattaaccagaggaagaatgtggatgacgacgatgagtctgagaacgatgacatgtacgagagcaccacagctggctcttctagtgaaacggacaacgagagcgatgaagaagagaatgacagcagtgacaacaataatagcgatgaggaacagatagagaactccaacaacaataattctgacgaggaacagaatgactcctcttccaacgacaataatgatgaggagaatgaggaacaggacgacgtcatggacaatgaccaaaacgataagaagatcaagcatagcttcaatctcgccaacgagagtaaacacactaaagaggaacgagtgaaagaagagaaaaagctgaagatctatgacttcataaacgacaaggagaaaagacttaactttaatggcgatcagaaagatgaagataacgaggagaacgatgataaagatgagaacacgcttgagaatcggaatatcatctccaaacacacttcagtgtttcctggcctgtacttcatcgggattgggtataacctcctcttcgggaaccccttgggtgaagctgattcccttatcgatccaggttatcgggcgcaaatttacctgatggaatgggctctcagcaaggaaggcattgccaacgacctgagcactctgcaacccgtgaatggatggatacgaaaggagaatgcctgctccagagttgaatctattacagaatgcagctctatatctgactacaccaaatccctgtcagcggaggcaaaggttagtggctcttattggggaatcgcctccttctcagcatccaccgggtatagctcttttctccacgaggtgacaaaacgcagcaagaaaaccttcctcgtgaaatccaactgtgtgaaatacactatcgggcttcctccctatattccctgggacaagaccacggcctacaagaatgccgtaaatgaactgccagctgtatttaccggtttggataaagaatccgaatgtccctctgatgtgtacgaagagaacaagacaaaatcaaactgcgagaacgtgagtctgtggatgaagttcttcgacatttatggcactcacatcatttatgaaagtcaattgggaggaaagataacaaagattatcaatgtttccacctcaagtattgagcagatgaagaaaaatggagtctcagtcaaagcgaaaattcaagcacagtttgggtttggttcagccggtggctcaaccgacgtgaatagcagtaactcctccgcaaatgatgagcagtcttacgacatgaatgaacagctgatcgtcataggagggaatccgatcaaagacgtcaccaaggaggagaatctgtttgagtggtctaaaactgtgacaaaccatcctatgccgatcaacattaaactgactcccattagcgacagttttgactcagacgacctgaaagaatcctatgataaagccatcatctactattctcgcctgtacggactgtcccctcatgacacaatgcagaaggatgacaaggatattatcaagatcctgaccaacgctgatacggttaccaagaactcagctcctccaatcaacgctcagtgtcctcatgggaaagtcgttatgttcggattcagcctgaagcagaatttctgggacaacaccaacgcactcaaaggatacaacatcgaagtctgcgaagcagggagcaattcttgcactagcaaacaagggagcagcaataaatacgatacatcttacctttacatggaatgtggcgatcagccattgccctttagcgagcaggtgattagcgagagtacaagtacctataataccgtgaaatgtccgaatgattactccattctcctgggcttcggaatatcaagtagctcagggaggataaatagcgctgaatatgtctacagcacaccatgtattcccggcatgaagtcctgcagcctcaatatgaataatgacaaccagaagtcatacatctacgtgctgtgtgtagatactacgatctggagtggcgtgaacaacctgagcctggttgcccttgatggcgcacatggtaaggtaaaccgcagtaagaagtactccgacggtgaactggttggcacctgtccactggacggcacagtcctgactggatttaaggttgagtttcacacttcatctccatatgtgcagacacctttcgagaaatgcgccaaaagcttgaaagcctgctccgtacatggctccggtcacgccattggcattcagaactttaagtcactgttcatatacatgttgtgcaagaacaataagtaa

SEQ ID NO: 52 - protein, 843 aa, Plasmodium falciparum
  misc_feature: PfSPECT2
MAKLRILKKHYYVVFILLYLYDISCFKCIRLNNRSIYKNKYKNNVHIGTNENIRSIEKYSNVLCNSILCKNDKISSFINQRKNVDDDDESENDDMYESTTAGSSSETDNESDEEENDSSDNNNSDEEQIENSNNNNSDEEQNDSSSNDNNDEENEEQDDVMDNDQNDKKIKHSFNLANESKHTKEERVKEEKKLKIYDFINDKEKRLNFNGDQKDEDNEENDDKDENTLENRNIISKHTSVFPGLYFIGIGYNLLFGNPLGEADSLIDPGYRAQIYLMEWALSKEGIANDLSTLQPVNGWIRKENACSRVESITECSSISDYTKSLSAEAKVSGSYWGIASFSASTGYSSFLHEVTKRSKKTFLVKSNCVKYTIGLPPYIPWDKTTAYKNAVNELPAVFTGLDKESECPSDVYEENKTKSNCENVSLWMKFFDIYGTHIIYESQLGGKITKIINVSTSSIEQMKKNGVSVKAKIQAQFGFGSAGGSTDVNSSNSSANDEQSYDMNEQLIVIGGNPIKDVTKEENLFEWSKTVTNHPMPINIKLTPISDSFDSDDLKESYDKAIIYYSRLYGLSPHDTMQKDDKDIIKILTNADTVTKNSAPPINAQCPHGKVVMFGFSLKQNFWDNTNALKGYNIEVCEAGSNSCTSKQGSSNKYDTSYLYMECGDQPLPFSEQVISESTSTYNTVKCPNDYSILLGFGISSSSGRINSAEYVYSTPCIPGMKSCSLNMNNDNQKSYIYVLCVDTTIWSGVNNLSLVALDGAHGKVNRSKKYSDGELVGTCPLDGTVLTGFKVEFHTSSPYVQTPFEKCAKSLKACSVHGSGHAIGIQNFKSLFIYMLCKNNK

SEQ ID NO: 53 - protein, 842 aa, Plasmodium falciparum
  misc_feature: SPECT2
MKLRILKKHYYVVFILLYLYDISCFKCIRLNNRSIYKNKYKNNVHIGTNENIRSIEKYSNVLCNSILCKNDKISSFINQRKNVDDDDESENDDMYESTTAGSSSETDNESDEEENDSSDNNNSDEEQIENSNNNNSDEEQNDSSSNDNNDEENEEQDDVMDNDQNDKKIKHSFNLANESKHTKEERVKEEKKLKIYDFINDKEKRLNFNGDQKDEDNEENDDKDENTLENRNIISKHTSVFPGLYFIGIGYNLLFGNPLGEADSLIDPGYRAQIYLMEWALSKEGIANDLSTLQPVNGWIRKENACSRVESITECSSISDYTKSLSAEAKVSGSYWGIASFSASTGYSSFLHEVTKRSKKTFLVKSNCVKYTIGLPPYIPWDKTTAYKNAVNELPAVFTGLDKESECPSDVYEENKTKSNCENVSLWMKFFDIYGTHIIYESQLGGKITKIINVSTSSIEQMKKNGVSVKAKIQAQFGFGSAGGSTDVNSSNSSANDEQSYDMNEQLIVIGGNPIKDVTKEENLFEWSKTVTNHPMPINIKLTPISDSFDSDDLKESYDKAIIYYSRLYGLSPHDTMQKDDKDIIKILTNADTVTKNSAPPINAQCPHGKVVMFGFSLKQNFWDNTNALKGYNIEVCEAGSNSCTSKQGSSNKYDTSYLYMECGDQPLPFSEQVISESTSTYNTVKCPNDYSILLGFGISSSSGRINSAEYVYSTPCIPGMKSCSLNMNNDNQKSYIYVLCVDTTIWSGVNNLSLVALDGAHGKVNRSKKYSDGELVGTCPLDGTVLTGFKVEFHTSSPYVQTPFEKCAKSLKACSVHGSGHAIGIQNFKSLFIYMLCKNNK

SEQ ID NO: 54 - DNA, 2541 bp, Artificial sequence
  Synthetic sequence
  misc_feature: PvSPECT2 human CO
gccaccatggctaagccaagaaatataaactctctgttggccatatggtgtattctcttctcaatctgtgagtacggctatgtgggatctttgagaataggtcttcggagaaactatagtcaaggggactcctctgatcggctcgggggtagtgacgtggacgtgagacggtccggcgagcttatggacctcggtaagaacgcaaacatcctctgcaataacatcagctgtaacagtaaaaaagaggcttcttttctgagtcagaaaaaacaacttgaggatgacgatgacgacgagctggccgcgctgtacaatgatgatgatgacgatgctagcacaaccactggggggggttcagaaactagcgatgacgacgagcttgaactccctgatcaggacgaaggtgcggacgagggtgaagcagaggaccaactcgcgacacaggaggattccgacaactcagggaccgaccaaggtctgaaaaaaaaagtaaaccttagccgccacgaaaagttgatagaagacaaaaaacaacagacagagaatacttttaagaagtacaggtttggggatgaggaggaagagtcagaggagaaatccccggggaaatctaagtcactcgatccaagcagcctcgatgatgacgacggcgaaggtgatgatgatgatgatggcgacgagagagaaaagaagcaaagtaatacaaggaaagccatgaagaaggacttggatgttttcccagggctttactttgcaggcatcggatacgatagcctcttcggaaaccccctgggcgaggctgacagcttgaccgatcccgggtaccggggccaaatcatactcatgaactgggaactctcaaataagggagttgcgaatgatcttgcgacattgcaacctctgaatggatggatccgcaaggaaaatgcgtgcagtcgagcagaatctataaaagagtgttcttcagtatcagactacacgaaaaatttgaccgcagaggcatctgtatctggctcctacatgggctttggagcttttagtgctagtactggttataagaaatttttgcaagaagccagtaaacgcacgtccaaaacttatctggtaaaaagcaattgcgtgaaatacacggtcgggctccctccttatgtgcgctgggagcagacgaccgcctttaagaacgctgtgaacgggttgccaccgcatttcaccggactggaggccgactctgaatgcgcctctgatgtctacgaacaaaagaagacatctgaagaatgcgaaaccgtacatgcctggatacgcttttttaagacttatggaacacacgtgatcatggaagcacagcttggcggcaaaataacaaagattatccgggtcgagaatagctccgtcaatcaaatgaagaaagacggagtcagcgtgaaagcccagatcaaagcccaattcggctttgcatctgtggggggaagcacaaacgtctctagtgaccatagttcaaaaaagaacgaggataattatgagatgtctgagcagctggtagtgattggaggcaacccaatcaaggatgtgacgaaagaagaaaatctctatgagtggtctaaaacggtctcaacaaatccgatgcctatcaatatcaagctcctgccgattagcactatctttgactccgacgatctgaaaaatagttacgaaaaggcattgatctactacactcgcttgtatggcttttcaccccatgatacaatgcaaaaagatgaaaaggatattgttaagatcctgacggccagcaccacagtcacaaagacaggcccgccgcctatatctgcagagtgtccgcataatatggtggtcttgttcggtttcgtagtcaaacagaatttttgggatcatacgaacaaactccagagttatgaaatggagatttgtgaaagcggtgccagctcatgcacgtctaagcagggaaatacaaataagtatgacgtatcctatacgtatattgagtgcggaccacaggcattgccgttcactgagcaggttgtatctgtatccggtacaacatataactctgttaaatgcccgaatgactacagcgtgttgtttggctttggaatggctacgagttctggcaggcaccaaagtgcgctttatagctatttcacaccatgtcgaccagggctcaaaagctgtagcttgaatatgaacgaacatgatgataagtcttacatttacctggtctgcgtcgacgcgactatctggacgggacttaacgcgttgagcatgatcgcgaaagacgatttgcacagtgccgtgaaccggtaccaacaattcaatgatggagagctggttgtgacgtgtcctagcgagggcactatactgactggattctatggggagacccatacctccagtccatatgttactgtaccttttgggaaatgcgcaaagtcattgaaagcctgttctgttcatgggtccggccaagccataggcattcacaattatagaactttgttcacagtcgcactgtgtaagaataattag

SEQ ID NO: 55 - protein, 844 aa, Plasmodium vivax
  misc_feature: PvSPECT2
MAKPRNINSLLAIWCILFSICEYGYVGSLRIGLRRNYSQGDSSDRLGGSDVDVRRSGELMDLGKNANILCNNISCNSKKEASFLSQKKQLEDDDDDELAALYNDDDDDASTTTGGGSETSDDDELELPDQDEGADEGEAEDQLATQEDSDNSGTDQGLKKKVNLSRHEKLIEDKKQQTENTFKKYRFGDEEEESEEKSPGKSKSLDPSSLDDDDGEGDDDDDGDEREKKQSNTRKAMKKDLDVFPGLYFAGIGYDSLFGNPLGEADSLTDPGYRGQIILMNWELSNKGVANDLATLQPLNGWIRKENACSRAESIKECSSVSDYTKNLTAEASVSGSYMGFGAFSASTGYKKFLQEASKRTSKTYLVKSNCVKYTVGLPPYVRWEQTTAFKNAVNGLPPHFTGLEADSECASDVYEQKKTSEECETVHAWIRFFKTYGTHVIMEAQLGGKITKIIRVENSSVNQMKKDGVSVKAQIKAQFGFASVGGSTNVSSDHSSKKNEDNYEMSEQLVVIGGNPIKDVTKEENLYEWSKTVSTNPMPINIKLLPISTIFDSDDLKNSYEKALIYYTRLYGFSPHDTMQKDEKDIVKILTASTTVTKTGPPPISAECPHNMVVLFGFVVKQNFWDHTNKLQSYEMEICESGASSCTSKQGNTNKYDVSYTYIECGPQALPFTEQVVSVSGTTYNSVKCPNDYSVLFGFGMATSSGRHQSALYSYFTPCRPGLKSCSLNMNEHDDKSYIYLVCVDATIWTGLNALSMIAKDDLHSAVNRYQQFNDGELVVTCPSEGTILTGFYGETHTSSPYVTVPFGKCAKSLKACSVHGSGQAIGIHNYRTLFTVALCKNN

SEQ ID NO: 56 - protein, 843 aa, Plasmodium vivax
  misc_feature: SPECT2
MKPRNINSLLAIWCILFSICEYGYVGSLRIGLRRNYSQGDSSDRLGGSDVDVRRSGELMDFGKNANILCNNISCNSKKEASFLSQKKQLEDDDDDELAALYNDDDDDASTTTGGGSETSDDDELELPDQDEGADEGEAEDQLATQEDSDNSGTDQGLKKKVSLSRHEKLIEDKKQQTENTFKKYRFGDEEEESEEKSPGKSKSLDPSSLDDDDGEGDDDDDGDEREKKQSNTRKAMKKDLDVFPGLYFAGIGYDSLFGNPLGEADSLTDPGYRGQIILMNWELSNKGVANDLATLQPLNGWIRKENACSRAESIKECSSVSDYTKNLTAEASVSGSYMGFGAFSASTGYKKFLQEASKRTSKTYLVKSNCVKYTVGLPPYVRWEQTTAFKNAVNGLPPHFTGLEADSECASDVYEQKKTSEECETVHAWIRFFKTYGTHVIMEAQLGGKITKIIRVENSSVNQMKKDGVSVKAQIKAQFGFASVGGSTNVSSDHSSKKNEDNYEMSEQLVVIGGNPIKDVTKEENLYEWSKTVSTNPMPINIKLLPISTIFDSDDLKNSYEKALIYYTRLYGFSPHDTMQKDEKDIVKILTASTTVTKTGPPPISAECPHNMVVLFGFVVKQNFWDHTNKLQSYEMEICESGASSCTSKQGNTNKYDVSYTYIECGPQALPFTEQVVSVSGTTYNSVKCPNDYSVLFGFGMATSSGRHQSALYSYFTPCRPGLKSCSLNMNEHDDKSYIYLVCVDATIWTGLNALSMIAKDDLHSAVNRYQQFNDGELVVTCPSEGTILTGFYGETHTSSPYVTVPFGKCAKSLKACSVHGSGQAIGIHNYRTLFTVALCKNN

SEQ ID NO: 57 - DNA, 2478 bp, Plasmodium berghei
  misc_feature: GPI_P113
ggatccgccaccatggctaagatcttcctgtttagtttcttcttcgtatggttccagtattgctattctaagaacccctcagactacgctcatagcattgtctctaattttgagtccgagaatacgttgaagtgtttgaaaggcaacgtgtatatacttcaatgtcagatcaagtgcatgaactcaaacaacgagatcatatacaaggagtgtctcaacgatatcgaaaagatttgcaaagataagaaaacatgcagttattacttcgactacatcttcaaaaccaagaatcacaagcttagaaacaacaacaataacaacatctacatcgacaattgcatcgattccgacaagaatgagatcaaatctaccttcacctgtgtcctcaatccattgctggaatttgacaataacaaccacgtcatttataacttcctgctgaacaacaagaacaatgacaagatcgtctgtaaaaatagcaacatatatattaacaacgctactattcactataccttctccgacattaagtttaaagacgtgactagctacataaaagagaaatgtaacgagaaaactaattgcgtgataaacccctattctatccaaaccgatatcctgaatgagaagaacgacgcatatctgctgaactcatacatcagcatatccttcgcatgtgtgaaaattaacctggaaagctatctgtacggaggtgatatcgatgaatttgatcagataaacgatgaagaaaatgaggacaataagtacctggatcacaatgatctcgatgaaaaaaatgaggaaattatttccctgaagaacgaaatcaacgacatcctgaatgacgagaagatcgataacatcgccgagaagctgaagattgcgaaatttacaataagcaagaaaatcaacgaggagatcaagaagaagaacgacatctttaacaacctggccaacgacatctaccagttcatcggtaacgagtactactttacttccgacatcaaagacatgattgaagataggtacaatgaactgaacaaaacatctcagtctgacttgtattacatttaccttctgaatgtgtttgacattgaaaagatttacgggatctacctgtctagttatcaggagcgactgcagcagatcctgcaaaccaacatgacaaatctggactatgttgagaagaagattggaagcttgcggaacatttacatgttcctgtataaaaagagtaagaagtataacgcgctggatatcttcgacgaatactacgattatgtgctcaattacaatgacttcgctaaagacaatgagataatcagcgccgacattttcattaagtcaaaacctgatatccctcaactcaatttcgagatcaacaatgagaacaagaacgtgaagtataaagacgttaccgatcttgacgagctggataacctcaacaggataaatagaattatcaatatccggaatgtgctggtcaagcagctcaagatcctttacaaccagagaaataacatatttatcaaacaggccatgctggtgaaatcatactgctacaaaaatccactggatattacggatttcagcagcatcttcaagaataactacaataagttgaaatacgacgcctataaggagggcaatggacacattaacgtggccgacaaaatcaatccaaactttgtcgtgaaatacctgaacaatctttataaacagcatgttaacaagaattatatcctgaacagctgggaccctaaatacaatcgcatgaataagaagattaagatcattctcattttggggtatggccaggtaatccagatcgagaaacagattaaccgtcatataggcaagtacaacgccctgcttgaaaaggcaaccctctataacgtgggaaatctctttacacagactacgaatattttgaacgacatctcaggctcactgaacgatgggcttgacccgaacatccatgatcaggaggatgtcactgtcgttgaatcctctgaaagtaacaagctcgcagaacctgaggaacccattgagaaggtagaggttgatagggtagagaaatccgatgatgccaataatgccactcaacaagtgacaggaacagacgaggccaattacgatacagctagcgggaatagtaccaacatcaacattgacaacatagactatggtgtggacgaatctatccgaattattaagtactccaaggctgaggaggatgaatataatgagagcggcaataacgaaaatgaaaacaacgagaataacgagaacgagaacaatgaaaatgagaacaacgagaatgagaataatgagaatgaaaataacgagaacgaaaatatagaactgaagaatatagaacacgaaaacaaatccaacgcatctagcgcttccctcagtaacattttctttacctttatcattgcagctctgtttatccgcccctttctgtgactcgag

SEQ ID NO: 58 - protein, 819 aa, Plasmodium berghei
  misc_feature: GPI_P113
MAKIFLFSFFFVWFQYCYSKNPSDYAHSIVSNFESENTLKCLKGNVYILQCQIKCMNSNNEIIYKECLNDIEKICKDKKTCSYYFDYIFKTKNHKLRNNNNNNIYIDNCIDSDKNEIKSTFTCVLNPLLEFDNNNHVIYNFLLNNKNNDKIVCKNSNIYINNATIHYTFSDIKFKDVTSYIKEKCNEKTNCVINPYSIQTDILNEKNDAYLLNSYISISFACVKINLESYLYGGDIDEFDQINDEENEDNKYLDHNDLDEKNEEIISLKNEINDILNDEKIDNIAEKLKIAKFTISKKINEEIKKKNDIFNNLANDIYQFIGNEYYFTSDIKDMIEDRYNELNKTSQSDLYYIYLLNVFDIEKIYGIYLSSYQERLQQILQTNMTNLDYVEKKIGSLRNIYMFLYKKSKKYNALDIFDEYYDYVLNYNDFAKDNEIISADIFIKSKPDIPQLNFEINNENKNVKYKDVTDLDELDNLNRINRIINIRNVLVKQLKILYNQRNNIFIKQAMLVKSYCYKNPLDITDFSSIFKNNYNKLKYDAYKEGNGHINVADKINPNFVVKYLNNLYKQHVNKNYILNSWDPKYNRMNKKIKIILILGYGQVIQIEKQINRHIGKYNALLEKATLYNVGNLFTQTTNILNDISGSLNDGLDPNIHDQEDVTVVESSESNKLAEPEEPIEKVEVDRVEKSDDANNATQQVTGTDEANYDTASGNSTNINIDNIDYGVDESIRIIKYSKAEEDEYNESGNNENENNENNENENNENENNENENNENENNENENIELKNIEHENKSNASSASLSNIFFTFIIAALFIRPFL

SEQ ID NO: 59 - protein, 818 aa, Plasmodium berghei
  misc_feature: P113
MKIFLFSFFFVWFQYCYSKNPSDYAHSIVSNFESENTLKCLKGNVYILQCQIKCMNSNNEIIYKECLNDIEKICKDKKTCSYYFDYIFKTKNHKLRNNNNNNIYIDNCIDSDKNEIKSTFTCVLNPLLEFDNNNHVIYNFLLNNKNNDKIVCKNSNIYINNATIHYTFSDIKFKDVTSYIKEKCNEKTNCVINPYSIQTDILNEKNDAYLLNSYISISFACVKINLESYLYGGDIDEFDQINDEENEDNKYLDHNDLDEKNEEIISLKNEINDILNDEKIDNIAEKLKIAKFTISKKINEEIKKKNDIFNNLANDIYQFIGNEYYFTSDIKDMIEDRYNELNKTSQSDLYYIYLLNVFDIEKIYGIYLSSYQERLQQILQTNMTNLDYVEKKIGSLRNIYMFLYKKSKKYNALDIFDEYYDYVLNYNDFAKDNEIISADIFIKSKPDIPQLNFEINNENKNVKYKDVTDLDELDNLNRINRIINIRNVLVKQLKILYNQRNNIFIKQAMLVKSYCYKNPLDITDFSSIFKNNYNKLKYDAYKEGNGHINVADKINPNFVVKYLNNLYKQHVNKNYILNSWDPKYNRMNKKIKIILILGYGQVIQIEKQINRHIGKYNALLEKATLYNVGNLFTQTTNILNDISGSLNDGLDPNIHDQEDVTVVESSESNKLAEPEEPIEKVEVDRVEKSDDANNATQQVTGTDEANYDTASGNSTNINIDNIDYGVDESIRIIKYSKAEEDEYNESGNNENENNENNENENNENENNENENNENENNENENIELKNIEHENKSNASSASLSNIFFTFIIAALFIRPFL

SEQ ID NO: 60 - DNA, 2919 bp, Artificial Sequence
  PfP113 human CO
gccaccatggctaagatcccgttctttattctgcacatcctcctcctgcagtttctgctttgtctgatacgctgttatgtgcacaatgatgtgattaagttcggtgaggagaatagcctgaagtgctctcagggaaacttgtatgtgttgcactgtgaggtgcagtgccttaatggcaataatgagattatccacaagaggtgtaatgacgacattgagaaaaagtgcaacggcaataataaatgcatatacttctttgagtacgaactgcggaagaaaacacaaagctttcgaaataagaattctatcgagatttccgagtgtgtcgaaagcgagcagaacgaagtgaaaacctcaactacctgtctcctgagcaattcctttattcttgacgaggcctttatacagtatttcttcttcataaagaacaagaacgaagagcctgtgatttgtaaggatgggaatatcaacattaagagtgcactcctgcactctccgttctgtgaaatcaaactcaaggacatttccgaatatatacgcaaaaagtgtgacaacaacaaggaatgccttatagatcctctcgatgttcagaagaatttgctgaacgaagaagatccctgctacatcaataacagttacgtatctgtgaatgtggtctgcaacaaagaggaggagataggggatgagagcactgacagctcatcaatggagatccaggactcaacatcaaatgagcaagacgagaatgttaaaggaatgtcaagcagccaagagatgaactcaaacaacgatgaaaacaaaaaccaagacaacgaaagcgacgatgacgtcaataataataacaacaataacaatgatgaccaggacgagcaaggaaacgatggcgatgtcaccagctctatgaacaagaatgaggacaacaaggatttggagcatggttcctccaatgatgtcaataacaacactgacaccttggttaacaacaaagagaataaggagttcgtccttaaagagaagtctagccttacttctaaaattaacaaagagctggctcatagaactgccctgtttaacaaacttgcagacaacatatcacttctgcttaacaagaaatacgattccttcgaaattaaggatgtgctggaagataggtacaacgagatgaagagggacgcaaaccccgatgtctactacatatacctgatggatactctggatattgaaaagatcgaagatatcaacctggaagaggttaagatgtctctgctggcatcactgaaagaaacgatgaacaaaattgatacgatcgaaaagaaaatcgaagaatttaagaacaagtacatctccttgtataacaaggtgaaaaccacaatgcccgaactctttgacctgaatgaggatcttgtactgctctacaacgattttccctttgacaacggcatgatcagctccgacatcttctttaagtacaatccttccgagaacatcatggatcatcaggaaatggtgaagaaagggagtatcaccgaagatgaactcaggattgttaacgatcttgagccactggataactatagacgtcgtaaacgaattacagagctgagaaagattctggtggagaagctgcggattctgtacctggagaagaacaatctcttcaatacacaggcgagttgcatcaagagctattgctataagaatcctctgaacctcaaaaccttggaagtgctcttgaagaagaactactatagactgaaggagaataaagattacgatgttgtatccagcattatccagcatctcgacaatgtagacgccaacaagaagaagaaatggctgacccatgaacggatactgaagaagctccaagttctgattgccgaaggctataagcggatcaacgagaaagaaaaggacatcgaccgaagaatggctgtctacaatgccctctatgagaaagcacagtcttacaacctgcagaagcttttcaacgactccaacgattttctgaagaaatatgccataatgggaaacagtttcgacgacggcgatgaggttttcggttcccaaagctcaaactttaacatcttcgatagcaataacaccgaccagaacaatgagcaagagcagccaaagcaagatgaccagcttttgaacaataataacgatgacgtgctctcagagtcaaacaatgagaataaagagaaaacaagtgatgacgctactcataaggagactcaggagaaaagcgaccaggaaccttcccagaacattcaggaggacaactccgatgagaaacatgccgaaaacgaggagaacgtagaacagatcgaaactgatagtaatgtcagcgaagaagccaatgacgagaataaggataacatgcagacaaccactgacgaaggaaccgaagaacttcagcagaatgacgaagatgcggagagtctgaccaaggagaattccaaatctgaggagcaggagaatgaagattctactgacgccgaggcgattgataaagaggaagttgaaacggaagagaagggaaaggacgaacagaagaaagatgagcagaaggagcaggatgaggaagaggatggagagaaagaaaataagcacaagagctccgaaactaccaatgagactgtgaccgacatcgaggaaaataagaacgaggtcaaaggtgaggagcatcttcaggggtctgagcagagcattgaggcttccgaatcatcccagaaagatgagactaaagaaacagaggacaaggaggaatacgtgaacgctaatgatgacgaatcttctgaggaggacacgacgcctaacgagacaaataaaaccgacaacgggtcaagtttcttcttcgcgatgagcaatgcacttctcgtgatcttgctgttgcttttcatcgaattcctgtaa

SEQ ID NO: 61 - protein, 970 aa, Plasmodium falciparum
  misc_feature: PfP113
MAKIPFFILHILLLQFLLCLIRCYVHNDVIKFGEENSLKCSQGNLYVLHCEVQCLNGNNEIIHKRCNDDIEKKCNGNNKCIYFFEYELRKKTQSFRNKNSIEISECVESEQNEVKTSTTCLLSNSFILDEAFIQYFFFIKNKNEEPVICKDGNINIKSALLHSPFCEIKLKDISEYIRKKCDNNKECLIDPLDVQKNLLNEEDPCYINNSYVSVNVVCNKEEEIGDESTDSSSMEIQDSTSNEQDENVKGMSSSQEMNSNNDENKNQDNESDDDVNNNNNNNNDDQDEQGNDGDVTSSMNKNEDNKDLEHGSSNDVNNNTDTLVNNKENKEFVLKEKSSLTSKINKELAHRTALFNKLADNISLLLNKKYDSFEIKDVLEDRYNEMKRDANPDVYYIYLMDTLDIEKIEDINLEEVKMSLLASLKETMNKIDTIEKKIEEFKNKYISLYNKVKTTMPELFDLNEDLVLLYNDFPFDNGMISSDIFFKYNPSENIMDHQEMVKKGSITEDELRIVNDLEPLDNYRRRKRITELRKILVEKLRILYLEKNNLFNTQASCIKSYCYKNPLNLKTLEVLLKKNYYRLKENKDYDVVSSIIQHLDNVDANKKKKWLTHERILKKLQVLIAEGYKRINEKEKDIDRRMAVYNALYEKAQSYNLQKLFNDSNDFLKKYAIMGNSFDDGDEVFGSQSSNFNIFDSNNTDQNNEQEQPKQDDQLLNNNNDDVLSESNNENKEKTSDDATHKETQEKSDQEPSQNIQEDNSDEKHAENEENVEQIETDSNVSEEANDENKDNMQTTTDEGTEELQQNDEDAESLTKENSKSEEQENEDSTDAEAIDKEEVETEEKGKDEQKKDEQKEQDEEEDGEKENKHKSSETTNETVTDIEENKNEVKGEEHLQGSEQSIEASESSQKDETKETEDKEEYVNANDDESSEEDTTPNETNKTDNGSSFFFAMSNALLVILLLLFIEFL

SEQ ID NO: 62 - protein, 969 aa, Plasmodium falciparum
  misc_feature: PfP113
MKIPFFILHILLLQFLLCLIRCYVHNDVIKFGEENSLKCSQGNLYVLHCEVQCLNGNNEIIHKRCNDDIEKKCNGNNKCIYFFEYELRKKTQSFRNKNSIEISECVESEQNEVKTSTTCLLSNSFILDEAFIQYFFFIKNKNEEPVICKDGNINIKSALLHSPFCEIKLKDISEYIRKKCDNNKECLIDPLDVQKNLLNEEDPCYINNSYVSVNVVCNKEEEIGDESTDSSSMEIQDSTSNEQDENVKGMSSSQEMNSNNDENKNQDNESDDDVNNNNNNNNDDQDEQGNDGDVTSSMNKNEDNKDLEHGSSNDVNNNTDTLVNNKENKEFVLKEKSSLTSKINKELAHRTALFNKLADNISLLLNKKYDSFEIKDVLEDRYNEMKRDANPDVYYIYLMDTLDIEKIEDINLEEVKMSLLASLKETMNKIDTIEKKIEEFKNKYISLYNKVKTTMPELFDLNEDLVLLYNDFPFDNGMISSDIFFKYNPSENIMDHQEMVKKGSITEDELRIVNDLEPLDNYRRRKRITELRKILVEKLRILYLEKNNLFNTQASCIKSYCYKNPLNLKTLEVLLKKNYYRLKENKDYDVVSSIIQHLDNVDANKKKKWLTHERILKKLQVLIAEGYKRINEKEKDIDRRMAVYNALYEKAQSYNLQKLFNDSNDFLKKYAIMGNSFDDGDEVFGSQSSNFNIFDSNNTDQNNEQEQPKQDDQLLNNNNDDVLSESNNENKEKTSDDATHKETQEKSDQEPSQNIQEDNSDEKHAENEENVEQIETDSNVSEEANDENKDNMQTTTDEGTEELQQNDEDAESLTKENSKSEEQENEDSTDAEAIDKEEVETEEKGKDEQKKDEQKEQDEEEDGEKENKHKSSETTNETVTDIEENKNEVKGEEHLQGSEQSIEASESSQKDETKETEDKEEYVNANDDESSEEDTTPNETNKTDNGSSFFFAMSNALLVILLLLFIEFL

SEQ ID NO: 63 - DNA, 3141 bp, Artificial Sequence
  PvP113 human CO
gccaccatggctaagctcccgcccctctgcaggttgccacttgcgctggtgcttctttgtctgacatccagagcgcgctgttatgtacacaacgacgtgatgaaatttggtgaagaaaactctctcaagtgttcacaagggtctctctacatccttcactgcgaggtcaagtgtgtgaatgcaaagaacagaatcatccaccgaagttgcatcgaccaggttgaggcgaagtgcatgggcaacgccaaatgtaagtactattttgactatgtcgtgaaatcccgcggacagagtttgcggaacaaaaatgaaatcgaaatagaagaatgcgtggaatccgaacggaatgagattaagacgtccacaacttgccttctgtcaaattcatttttgctggatgagacctacattcaatatttctttttcatcaaaaacaaaaatgaagaacccataacgtgtcgagacggtagactgagcgtaaagagtgcgatacttcatagtcccttttgcaaaataaatcttaaggacatcactgagattctcaagagacaatgcgatcacagtaaggagtgcgtcattaatccgtatgtcctccaaaaagatgcattgaatgagagagaccagtgttacattaacaactcatatgtctcactcaatgtcgtttgcaccaaagaaggggaggagcaaccagaagagagtggacataaacagaagagggatgacgacgtggacgaaaccgaggaaggctcatatgacgtctctgcggatcagaacaaatcagcaatagtcggcgagggaaacgatgatccggaatccctgggtgaagaagatgagctttcagagactaacgaggcagttgatctcattatgaactccaaggaaagctttgaaaacaaaattcggaaggcgaagtcaatacttctctcacagatgaacgagcaggaggtgaaaaagaatgcaatattcaagaaactcggggaagagctttccaaaatggtcctccagaaatatgaaccaagtgatctgaaagacttgatcgaggacaggtataatgaaatgagacggtcccctgaccaagacctttattatctctaccttatagatacactcgaaataaataaaatggaagatcttgacgtgactgcactgcaagaccagctcgctatattgttggaagagcagatgggcaagatgaatcgcattgagaaaacgattaaccgattgaggaaaaaatacctttctatttacaacaaagcgaagaacaagaaagttaaagacatttacgatgaaggagttgatcctgtactcacgtacgacgacttcgcgcacggcaacggcattattacggcagatattttctttaagtataagcctgccattaagccgttgacttttagcaaatctaatgcctctgaggagcgggggtcatccaaaaaaaaagagtacaaggaccttcttgagatggacgcgctggatgagtacaaccggaagaaacggataactgacatgcgaaacggtctgatggagactttgaagaaaatgtattacgcaaaaaatgggatattcaataatctggcgagttgcattaagtcatattgctataaaaggcctctcaaccttaatgcactttcctccgttcttaaaagaaattttgaaaatcttcgagagaaaaaatcaaccgatccagtggcccccatagtcagatatttgcaaaaggtcagtggcgaggttggcggggaagtgggaggcgcggctggcggtgctgcggggggtgctgcaagtggtgccgcatcaggagctgtcagtgaagcagtaggaggcgccataggtggggctgtgggcgaggcatctatcgcagtaaatccgccccgatgggaaaaatcacgacgaattcttcaaaagctcaaggctttgctgcacctgggttatcaacaggcactggacaaagagttggagatcgacgagaggactgacaaatacaacgctctgaatgagaaagcaaaagaatataaccttcagcggcttttttccgagtcagacaagttgcttaagaaggtcgcaatgcttacgtcagccagtgaaagtgcagatgaagtattcggtaaccaagcgagctttttcgatgtttacagaggagaggcagcgtcacaaaaaggagtagccgccagtgaaaagggcgtagctgcaagagagaaaggagtcgccgcgagtgagaagggggtcgcggcatcagaaaagggtgtggcggccagcgagaagggtgtcgcggcatcagaaaaaggtgttgccgcaagccaaaaaggcggagaaacgtctgaggaaggggaggctgccagtcaaaaggatgttgctacgtccgaggagggtggagctagcagcgagaaacgcgatgacgacgagatgaatccgcccgacgaagggtatgagtccgctaaggaagacggggagaatgcacaagatgacgacagcggcggaaatgctgaacccgtagagggaaaagcaggcgaaagtgaagacgctgatggggtaaatgccggctccaataaagaaggtgaggatggggagagcgttgaagaagaagctgcggaaggggaagcggcacccaaggaggaggctgcagacggagaagatgctccggaggaggaagccgagggtgaggatgccccagaagaagaggccgatggcgaagacgccccgaaggaggaggctactgacggagaggacgcccctaaagaggaagaggccgaaggtgaggatgcaagcaaggacgaggaggccgacgaaggctcaactgacgaggaagaagccgccgacggcgggagtactgatgctacagcagccgatgaagctgcaggcggagtcgcggaccaaaacgacgtgcccgttaagggagaagactcagatggcgcggaatctgatggagcagaagatgccgccactgaaattaggggtgaggctgaagccggtgaagaggccgcggagcaacctactggagaggccgtagtcaaaggggattccgagggcggggcttctgggctggaaaccgagaagaagggagacgacgggggctccttcttccaaggtctttcccgagtgttgcttactgttcttgcaatactttccttggaatttctcctttga

SEQ ID NO: 64 - protein, 1044 aa, Plasmodium vivax
  misc_feature: PvP113
MAKLPPLCRLPLALVLLCLTSRARCYVHNDVMKFGEENSLKCSQGSLYILHCEVKCVNAKNRIIHRSCIDQVEAKCMGNAKCKYYFDYVVKSRGQSLRNKNEIEIEECVESERNEIKTSTTCLLSNSFLLDETYIQYFFFIKNKNEEPITCRDGRLSVKSAILHSPFCKINLKDITEILKRQCDHSKECVINPYVLQKDALNERDQCYINNSYVSLNVVCTKEGEEQPEESGHKQKRDDDVDETEEGSYDVSADQNKSAIVGEGNDDPESLGEEDELSETNEAVDLIMNSKESFENKIRKAKSILLSQMNEQEVKKNAIFKKLGEELSKMVLQKYEPSDLKDLIEDRYNEMRRSPDQDLYYLYLIDTLEINKMEDLDVTALQDQLAILLEEQMGKMNRIEKTINRLRKKYLSIYNKAKNKKVKDIYDEGVDPVLTYDDFAHGNGIITADIFFKYKPAIKPLTFSKSNASEERGSSKKKEYKDLLEMDALDEYNRKKRITDMRNGLMETLKKMYYAKNGIFNNLASCIKSYCYKRPLNLNALSSVLKRNFENLREKKSTDPVAPIVRYLQKVSGEVGGEVGGAAGGAAGGAASGAASGAVSEAVGGAIGGAVGEASIAVNPPRWEKSRRILQKLKALLHLGYQQALDKELEIDERTDKYNALNEKAKEYNLQRLFSESDKLLKKVAMLTSASESADEVFGNQASFFDVYRGEAASQKGVAASEKGVAAREKGVAASEKGVAASEKGVAASEKGVAASEKGVAASQKGGETSEEGEAASQKDVATSEEGGASSEKRDDDEMNPPDEGYESAKEDGENAQDDDSGGNAEPVEGKAGESEDADGVNAGSNKEGEDGESVEEEAAEGEAAPKEEAADGEDAPEEEAEGEDAPEEEADGEDAPKEEATDGEDAPKEEEAEGEDASKDEEADEGSTDEEEAADGGSTDATAADEAAGGVADQNDVPVKGEDSDGAESDGAEDAATEIRGEAEAGEEAAEQPTGEAVVKGDSEGGASGLETEKKGDDGGSFFQGLSRVLLTVLAILSLEFLL

SEQ ID NO: 65 - protein, 969 aa, Plasmodium vivax
  misc_feature: P113
MKLPPLCRLPLALVLLCLTSRARCYVHNDVMKFGEENSLKCSQGSLYILHCEVKCVNAKNRIIHRSCIDQVEAKCMGNAKCKYYFDYVVKSRGQSLRNKNEIEIEECVESERNEIKTSTTCLLSNSFLLDETYIQYFFFIKNKNEEPITCRDGRLSVKSAILHSPFCKINLKDITEILKRQCDHSKECVINPYVLQKDALNERDQCYINNSYVSLNVVCTKEGEEQPEESGHKQKRDDDVDETEEGSYDVSADQNKSAIVGEGNDDPESLGEEDELSETNEAVDLIMNSKESFENKIRKAKSILLSQMNEQEVKKNAIFKKLGEELSKMVLQKYEPSDLKDLIEDRYNEMRRSPDQDLYYLYLIDTLEINKMENLDVTALQDQLAILLEEQMGKMNRIEKTINRLRKKYLSIYNKAKNKKVKDIYDEGVDPVLTYDDFAHGNGIITADIFFKYKPAIKPLTFSKSNASEERGSSKKKEYKDLLEMDALDEYNRKKRITDMRNGLMETLKKMYYAKNGIFNNLASCIKSYCYKRPLNLNALSSVLKRNFENLREKKSTDPVAPIVRYLQKKSRRILQKLKALLHLGYQQALDKELEIDERTDKYNALNEKAKEYNLQRLFSESDKLLKKVAMLTSASESADEVFGNQASFFDVYRGEAASEKGVAASQKGGETSEEGEAASQKDVATSEEGGASSEKRDDDEMNPPDEGYESAKEDGENAQDDDSGGNAEPVEGKAGESEDADGVNAGSNKEGEDGESVEEEAAEGEAAPKEEAADGEDAPEEEAEGEDAPKEEAADGEASSKEEETDGEVAPEEAADGEDAPKEEATDGEDAPKEEEAEGEDASKDEEADEGSTDEEEAADGGSTDATAADEAAGGVADQNDVPVKGEDSDGAESDGAEDAATEIRGEAEAGEEAAEQPTGEAVVKGDSEGGASGLETEKKGDDGGSFFQGLSRVLLTVLAILSLEFLL

SEQ ID NO: 66 - DNA, 654 bp, Plasmodium berghei
  misc_feature: Ag40
ggatccgccaccatggctggggcgagtatgagccacttgcagtgtctgacatctgttgctggtctgtcctctatcgtcatgtcaatgttccccaaactcattgccaataatccttccctgtttagaccactgctcaacatttcctggggatatctgttcggaagcactgtatggctgtgcttcttcagtgagattgggttggtcaggagaatcaatgctcctaaacggaagaatctgccagagaatgcagaacaagccaaagaacagctgaaggagatcaagaacaacgaaggcgattttaaccgacgcaatatcgacttcaagtacttctttagcctttccacaatcttctctagcatactgctgcttagcacagtgaaactcgccaacaacaatctgcagttgaggatctgttccaccattgtgtcactgagttgcatactgaacaatatgtactttcagaacaagatacactcacttgcactgaagaaagagagtctctttaaggacatgatcgatcgtccgaaagataccactattctggtgaacctgaagaagaacaagaccgactttcacatccatcatggcctttctctgctcttgctctatagcagcttctttggcctcactccctacattttcacgtgactcgag

SEQ ID NO: 67 - protein, 211 aa, Plasmodium berghei
  misc_feature: Ag40
MAGASMSHLQCLTSVAGLSSIVMSMFPKLIANNPSLFRPLLNISWGYLFGSTVWLCFFSEIGLVRRINAPKRKNLPENAEQAKEQLKEIKNNEGDFNRRNIDFKYFFSLSTIFSSILLLSTVKLANNNLQLRICSTIVSLSCILNNMYFQNKIHSLALKKESLFKDMIDRPKDTTILVNLKKNKTDFHIHHGLSLLLLYSSFFGLTPYIFT

SEQ ID NO: 68 - protein, 210 aa, Plasmodium berghei
  misc_feature: Ag40
MGASMSHLQCLTSVAGLSSIVMSMFPKLIANNPSLFRPLLNISWGYLFGSTVWLCFFSEIGLVRRINAPKRKNLPENAEQAKEQLKEIKNNEGDFNRRNIDFKYFFSLSTIFSSILLLSTVKLANNNLQLRICSTIVSLSCILNNMYFQNKIHSLALKKESLFKDMIDRPKDTTILVNLKKNKTDFHIHHGLSLLLLYSSFFGLTPYIFT

SEQ ID NO: 69 - DNA, 642 bp, Artificial Sequence
  PfAg40 human CO
gccaccatggctgggtgtacagtctctaatctcaaatgcgtgaccaatgtggcaggactggcaagtctggttatcagtctgtttccgaaactcatcataaagaacccacaagtgcttcgaccactgctgaatgtgtcctggggttatctgtttggtagcaccttttggctgtgcttcttctccgaagtaggactgcttcgcagcctgaagaacatgaaaggggtacctttgcctgaatcagccagtgaggcgaagaagcttctcgaagagatgaagaactctgagggcgatttcaatcggagatcactggacttccagtacttcttttccctcgctacgttgttctcaggcattctgttgctgagcacagtgaagttggccaaccataacctgcagcttaggcttagtagctctgtggtcgtcatcacatcactgctgaatagcctgtatctgcacaataaagtgcataatctgaaaagcaagaaagaaagcctctataacgactttattgccaatcccaagaacgagaaaactgtcgctgatctgaagaagaacaagaaagagtttcacatctttcacggattgtccgttctctctctctacgtttccttcttcggcctgactccctacattttcacctaa

SEQ ID NO: 70 - protein, 211 aa, Plasmodium falciparum
  misc_feature: PfAg40
MAGCTVSNLKCVTNVAGLASLVISLFPKLIIKNPQVLRPLLNVSWGYLFGSTFWLCFFSEVGLLRSLKNMKGVPLPESASEAKKLLEEMKNSEGDFNRRSLDFQYFFSLATLFSGILLLSTVKLANHNLQLRLSSSVVVITSLLNSLYLHNKVHNLKSKKESLYNDFIANPKNEKTVADLKKNKKEFHIFHGLSVLSLYVSFFGLTPYIFT

SEQ ID NO: 71 - protein, 210 aa, Plasmodium falciparum
  misc_feature: Ag40
MGCTVSNLKCVTNVAGLASLVISLFPKLIIKNPQVLRPLLNVSWGYLFGSTFWLCFFSEVGLLRSLKNMKGVPLPESASEAKKLLEEMKNSEGDFNRRSLDFQYFFSLATLFSGILLLSTVKLANHNLQLRLSSSVVVITSLLNSLYLHNKVHNLKSKKESLYNDFIANPKNEKTVADLKKNKKEFHIFHGLSVLSLYVSFFGLTPYIFT

SEQ ID NO: 72 - DNA, 639 bp, Artificial Sequence
  PvAg40 human CO + kozac
gccaccatgggggcgacggtatcatatcttagatgcgtgaccagtatagcagggctgagcagcctcgtgctgtctttgtttcccaagctgattatgaaaaaccctcaggtactccgaccactccttaacattagctggggttatttgtttggctcaaccttttggttgtgtttgttctccgaagtaggacttttccggtccctgaaaaatatgaagcgcataccaatccctgaaaacgcagaagaggctaagaagcaattggaggagatgaaaagcatggaaggggattttaccaggcgcagggaagatttccaatatttttttggtttttccaccttgttttctggtattcttcttctcagtacggtaagacttgcgaatcacaacatgcaactgaggatttccagtaccatcgttgcccttagctgcctgctcaataacttgtaccttcagaataaggtacattctcttaaaatccaaaaagaaaacctgtacaacgaactcatccgcaatcctaagtcagagacgactatagcggagattaagaaaaacaaaaaagatttccatatataccacggcttgtccctgttgtccctttacataagcttcctcggccttactccatatatatttacctag

SEQ ID NO: 73 - protein, 210 aa, Plasmodium vivax
  misc_feature: PvAg40
MGATVSYLRCVTSIAGLSSLVLSLFPKLIMKNPQVLRPLLNISWGYLFGSTFWLCLFSEVGLFRSLKNMKRIPIPENAEEAKKQLEEMKSMEGDFTRRREDFQYFFGFSTLFSGILLLSTVRLANHNMQLRISSTIVALSCLLNNLYLQNKVHSLKIQKENLYNELIRNPKSETTIAEIKKNKKDFHIYHGLSLLSLYISFLGLTPYIFT

SEQ ID NO: 74 - protein, 210 aa, Plasmodium vivax; identical to SEQ ID NO: 73
  misc_feature: Ag40

SEQ ID NO: 75 - DNA, 1077 bp, Plasmodium berghei
  misc_feature: Ag45
ggatccgccaccatggcttcttactcaaactcttccattaagcagaaatccgatagtgtgagtgtctacaatactcggactggaaatgtcagtaaaactcgcttgatccgtctgcaaaatgggcattaccgtagagtggtcgacattagcaataaggacgagaaggagattctcttcaggacatgtgcttgcgcttgtccaacacctcgaaatgaggagacacgcaaaacctatatgccacctctgaacaatgtgtctaccgtagcgtatagaaagcggatctattcttcctttgggaataaggacggtaacgatacaggcaacaacgagagcataacagaacatgaggacccgattaggaccttttccgaaacgacaagtaggcaggaaagtaccatcgacgacaaaacggagactagcatcaatagcaaggaaacagatgatggcaaccagtttggaaggttgtttgaagaactggaggagaaagaggatgaactgattgaggaaaaggaggaggagctgatagaagagaaggaggaggaacttatagaggagaaagaagaagagctgatcgaagagaaagaggaaatcacccctgagaacaaaaccctcataatgccctctaaaactctgatgaagggcattaagaccaacatttacttcctgtcaaacaaggaaaagatccaagtgcttatgtgctataactacaagtgtgatgccgttgtgttcgagaaagacacctttctgcgctatctctacatcaagagcatcaataatatcatcctgaacgaaagaatgattgaacagttgtgcaagaacgaaaacctgaagtacatccttgcctgcaacagcatagtggttgaatcaagcgacttcatcaaacccctgatcattgagtttgagtcatccacttccaagaacatcttcgtaaagcacattaagcacaatagccagaaagaaatggacatcaacaagttcaacgagtatatgcgggatctcaaaagcaatgagaagctcagactgaagaaagtcgagcgattccactctattaatctggcagccaagaaatgactcgag

SEQ ID NO: 76 - protein, 352 aa, Plasmodium berghei
  misc_feature: Ag45
MASYSNSSIKQKSDSVSVYNTRTGNVSKTRLIRLQNGHYRRVVDISNKDEKEILFRTCACACPTPRNEETRKTYMPPLNNVSTVAYRKRIYSSFGNKDGNDTGNNESITEHEDPIRTFSETTSRQESTIDDKTETSINSKETDDGNQFGRLFEELEEKEDELIEEKEEELIEEKEEELIEEKEEELIEEKEEITPENKTLIMPSKTLMKGIKTNIYFLSNKEKIQVLMCYNYKCDAVVFEKDTFLRYLYIKSINNIILNERMIEQLCKNENLKYILACNSIVVESSDFIKPLIIEFESSTSKNIFVKHIKHNSQKEMDINKFNEYMRDLKSNEKLRLKKVERFHSINLAAKK

SEQ ID NO: 77 - protein, 351 aa, Plasmodium berghei
  misc_feature: Ag45
MSYSNSSIKQKSDSVSVYNTRTGNVSKTRLIRLQNGHYRRVVDISNKDEKEILFRTCACACPTPRNEETRKTYMPPLNNVSTVAYRKRIYSSFGNKDGNDTGNNESITEHEDPIRTFSETTSRQESTIDDKTETSINSKETDDGNQFGRLFEELEEKEDELIEEKEEELIEEKEEELIEEKEEELIEEKEEITPENKTLIMPSKTLMKGIKTNIYFLSNKEKIQVLMCYNYKCDAVVFEKDTFLRYLYIKSINNIILNERMIEQLCKNENLKYILACNSIVVESSDFIKPLIIEFESSTSKNIFVKHIKHNSQKEMDINKFNEYMRDLKSNEKLRLKKVERFHSINLAAKK

SEQ ID NO: 78 - DNA, 1197 bp, Artificial Sequence
  PfAg45 human CO
gccaccatggcttcagattactttacaattctgtccaatattttcacaagcactagcctgaagaagaaatacagttcccggttgagcacaaaatccaagaagaaccaaaagcgagtcaaactgataagactgcggaatggacattttcgccgaattgtggatatttccaacattgacgagaagagcatcttccccagaagctgtacttttgcgtcaattagcagtgctagcaaagaaaacgagaggaagaattcaagcgaggacacaaaagaacctcaggagaatctgtatggcaaatcaaacacttcaagctctatcacgataaagatcaatttcgacgaaagcgatgagaacaagagtgatcaggataaccactctatcgataccattagcgacatctcttttacccagacttcacgcaaatctcttgaaattgaaagtaatacctatgagagttatcgcgaagtggagaaggaggacattgaggaggaggaggaagaggagaaagaagaagaatatgaggaggaagaagaagaagaggaatacgaagaggaagaggaagaagaggaggaagagtatgaggaggagggtctgaaaaccgaagaggagaaggaagaagataataaggaggtagagccagaagaggagcttaaagaagaagatgacaaggaggttgagcctgaggaggagaaggagaatgagcagaagaaagaagaacaagaggagaataacctcgaagctcccagcaaaacactgatgaaaggggttaagaccaacatatacttcctgtctaccaaagagcggatagaagcactcatgtgctacaactacatatccaacgccattattttcgaaaagggcaagtttctccgttatatcttcatgaacaatgtcaacaatatcatcgtgaacgagcacatgatcaatatgttgtgcaagaaggaaaagatcaaatacatcctgtcatctaactccatcatcattgaaagcaacgacttcatcaaaccgctcatcattgagtttgacagtaacatctctaagaagatctttgtcaaacacttgaaaatggtggactccttcaaactggatgacaagctgtacagggagtacctgaatgacctttctgaacatgagagggatagactgaaacatgtggagtccttctattccaatgccataaaggtgcacaatacgtaa

SEQ ID NO: 79 - protein, 396 aa, Plasmodium falciparum
  misc_feature: PfAg45
MASDYFTILSNIFTSTSLKKKYSSRLSTKSKKNQKRVKLIRLRNGHFRRIVDISNIDEKSIFPRSCTFASISSASKENERKNSSEDTKEPQENLYGKSNTSSSITIKINFDESDENKSDQDNHSIDTISDISFTQTSRKSLEIESNTYESYREVEKEDIEEEEEEEKEEEYEEEEEEEEYEEEEEEEEEEYEEEGLKTEEEKEEDNKEVEPEEELKEEDDKEVEPEEEKENEQKKEEQEENNLEAPSKTLMKGVKTNIYFLSTKERIEALMCYNYISNAIIFEKGKFLRYIFMNNVNNIIVNEHMINMLCKKEKIKYILSSNSIIIESNDFIKPLIIEFDSNISKKIFVKHLKMVDSFKLDDKLYREYLNDLSEHERDRLKHVESFYSNAIKVHNT

SEQ ID NO: 80 - protein, 395 aa, Plasmodium falciparum
  misc_feature: Ag45
MSDYFTILSNIFTSTSLKKKYSSRLSTKSKKNQKRVKLIRLRNGHFRRIVDISNIDEKSIFPRSCTFASISSASKENERKNSSEDTKEPQENLYGKSNTSSSITIKINFDESDENKSDQDNHSIDTISDISFTQTSRKSLEIESNTYESYREVEKEDIEEEEEEEKEEEYEEEEEEEEYEEEEEEEEEEYEEEGLKTEEEKEEDNKEVEPEEELKEEDDKEVEPEEEKENEQKKEEQEENNLEAPSKTLMKGVKTNIYFLSTKERIEALMCYNYISNAIIFEKGKFLRYIFMNNVNNIIVNEHMINMLCKKEKIKYILSSNSIIIESNDFIKPLIIEFDSNISKKIFVKHLKMVDSFKLDDKLYREYLNDLSEHERDRLKHVESFYSNAIKVHNT

SEQ ID NO: 81 - DNA, 1122 bp, Artificial Sequence
  PvAg45 human CO + kozac
gccaccatggctaatttgagttctcccctcctggccttgcccgaggagggtaagaagcgaagaacaaaactcatccgactgaggaacggtcactataggcggatagtggacatttcaaataccgacgaacggaagctcattccttctatgtgccgctgcgcgtgtgtcactcccagaaaagacgaagtggagaatgagggtaagtgggaagacgctaagaaagcaaaatcaagccaggaatatgatgaaacttctgattatgttgaatctgaaaagaaagagagctatatgctcgcagtcaatgaggaggatcagacggaggatatgtactcaaagacgatcagctttacttctataacccctacatctataagatccgaagagccagagccaaggcggaaactctcccttctggatgttaaagaagaagaggaagaggaggaggaggaagaagaagaggaagaagaggaagaggaggaagaggaagaggaagaagaggaagagaaagaaaaagaaaaagagaaggaggaagaggaagaagaggaggaggaagaggaagaggaagaggaggaggaagaagaagaggaagatgaaatagaatctaccgcagaggaaaaggaagaagagaagaagcaagtcccaccggaaggtaagaaattgatcgaaccctcaaagacccttatgagagggactaagaccaacatttattttttgagtaataaggagatggttcaaactctgatgtgttataattataattgcaacgcagtggtattcgaaaaagacacttttttgaggtatctgtacatgaagagcatcagcaacatcatcctcaacgaacgaatgatagatgaactgtgcaaacaggaagatcttaaatacgtgcttacaagcaatgctatcgtgttggaatctactgactttcttaagcctcttataattgagtttgaatccagtattagtaaaagagttttcgtgcgccacctgaagcataacgctcgcaaggaaatcgacatgaaaaaatatcatgattacatgggcgaacttaacgctaacgagaagattaggctgatgaaaattgagcgatttcatagtttcaacaagatgatccaatgcaactag

SEQ ID NO: 82 - protein, 371 aa, Plasmodium vivax
  misc_feature: PvAg45
MANLSSPLLALPEEGKKRRTKLIRLRNGHYRRIVDISNTDERKLIPSMCRCACVTPRKDEVENEGKWEDAKKAKSSQEYDETSDYVESEKKESYMLAVNEEDQTEDMYSKTISFTSITPTSIRSEEPEPRRKLSLLDVKEEEEEEEEEEEEEEEEEEEEEEEEEEEKEKEKEKEEEEEEEEEEEEEEEEEEEEEDEIESTAEEKEEEKKQVPPEGKKLIEPSKTLMRGTKTNIYFLSNKEMVQTLMCYNYNCNAVVFEKDTFLRYLYMKSISNIILNERMIDELCKQEDLKYVLTSNAIVLESTDFLKPLIIEFESSISKRVFVRHLKHNARKEIDMKKYHDYMGELNANEKIRLMKIERFHSFNKMIQCN

SEQ ID NO: 83 - protein, 345 aa, Plasmodium vivax
  misc_feature: Ag45
MNLSSPLLALPEEGKKRRTKLIRLRNGHYRRIVDISNTDERKLIPSMCRCACVTPRKDEVENEGKWEDAKKAKSSQEYDETSDYVESEKKESYMLAVNEEDQTEDMYSKTISFTSITPTSIRSEEPEPRRKLSLLDVKEEEEEEEEEEEEEEEEEEEEEEEKEEEEEEDEIESTAEEKEEEKKQVPPEGKKLIEPSKTLMRGTKTNIYFLSNKEMVQTLMCYNYNCNAVVFEKDTFLRYLYMKSISNIILNERMIDELCKQEDLKYVLTSNAIVLESTDFLKPLIIEFESSISKRVFVRHLKHNARKEIDMKKYHDYMGELNANEKIRLMKIERFHSFNKMIQCN

SEQ ID NO: 84 - DNA, 12 bp, Artificial Sequence
  Kozac consensus sequence
gccaccatggct

SEQ ID NO: 85 - DNA, 12 bp, Artificial Sequence
  Kozac consensus sequence
gccaccatggcc

SEQ ID NO: 86 - DNA, 6 bp, Artificial Sequence
  BamHI site
ggatcc

SEQ ID NO: 87 - DNA, 6 bp, Artificial Sequence
  Xhol site
ctcgag

SEQ ID NO: 88 - protein, 9 aa, Artificial Sequence
  CD8 T cell epitope
QAQRNLNEL

SEQ ID NO: 89 - protein, 9 aa, Artificial Sequence
  CD8 T cell epitope
SALLNVDNL

SEQ ID NO: 90 - protein, 9 aa, Artificial Sequence
  CD8 T cell epitope
KSPSNFTII

SEQ ID NO: 91 - protein, 9 aa, Artificial Sequence
  CD8 T cell epitope
SNQTNQETI

SEQ ID NO: 92 - protein, 9 aa, Artificial Sequence
  CD8 T cell epitope
ITPENKTLI

SEQ ID NO: 93 - protein, 9 aa, Artificial Sequence
  CD8 T cell epitope
KLIANNPSL

SEQ ID NO: 94 - protein, 9 aa, Artificial Sequence
  CD8 T cell epitope
SCILNNMYF

(19) United States
(12) Patent Application Publication
(10) Pub. No.: US 2020/0121775 A1
(43) Pub. Date: Apr. 23, 2020

(54) Functional screening of antigenic polypeptides-use for the identification of antigens eliciting a protective immune response and for the selection of antigens with optimal protective activity

(71) Applicants: INSTITUT PASTEUR, Paris (FR); CENTRE NATIONAL DE LA RECHERCHE SCIENTIFIQUE, Paris (FR)

(72) Inventors: Rogerio AMINO, Bagneux (FR); Anne-Sophie BEIGNON, Paris (FR); Pierre CHARNEAU, Paris (FR)

(21) Appl. No.: 16/483,028

(22) PCT Filed: Feb. 1, 2018
(86) PCT No.: PCT/EP2018/052551

§ 371 Date: Aug. 1, 2019

(30) Foreign Application Priority Data
Feb. 2, 2017 (EP) 17305121.0

(51) Int. Cl. A61K 39/015; C12N 15/10

(52) U.S. Cl. CPC A61K 39/015; A61K 2039/545; C12N 15/1082

(57) ABSTRACT

The invention is in the field of functional screening of protective antigens against pathogens in particular against pathogens such as parasites, bacteria, viruses or protective antigens of cancer cells in order to identify antigens suitable for the elicitation of a protective immune response in a host, and/or to optimize antigen design, in particular to define suitable antigen combinations for a protective immune response. The invention thus involves using a lentiviral vaccine platform for induction of a specific cellular or humoral response against assayed antigens on one hand, and using a fast and reproducible assay for the evaluation of the induced protective effect after challenge.

The invention is in the field of functional screening of protective antigens against pathogens in particular against pathogens such as parasites, bacteria, viruses or protective antigens of cancer cells in order to identify antigens suitable for the elicitation of a protective immune response in a host, and/or to optimize antigen design, in particular to define suitable antigen combinations for a protective immune response.

The invention thus involves using a lentiviral vaccine platform for induction of a specific cellular or humoral response against assayed antigens on one hand, and using a fast and reproducible assay for the evaluation of the induced protective effect after challenge.

The invention relates to a method of screening antigenic polypeptides and selecting them according to their capability to elicit a protective immune response against pathogens and cancer cells. Candidate pathogens for antigen testing encompass extracellular and intracellular viruses, bacteria, fungi, protozoans and worms.

In a particular embodiment the invention is directed to a method of functionally screening Plasmodium antigenic polypeptides and to a method of selection of antigenic polypeptides of malaria parasite that exhibit a protective effect, especially a protective immune response in a host challenged with Plasmodium sporozoites. This embodiment provides an illustration of the ability of the designed method and related tools to extend identification of individual protective antigens of pathogens or cancer cells. Importantly, after identifying individual protective antigens, the invention allows testing and selecting multi-antigenic combinations with effective, preferably optimal, protective activity against pathogens or cancer cells.

Malaria is causally related to infection of a host by a parasite and has been for many decades the object of extensive researches with a view to identify agents effective in the treatment of the clinical symptoms associated with the infection and protective vaccines. In the last 15 years, malaria control measures reduced by 48% the global deaths caused by this mosquito-bome disease. Despite this significant decrease in mortality, the WHO estimated ˜215 millions of malaria clinical episodes, resulting in more than 400,000 deaths in 2015. Actual malaria control programs rely mainly on the use of insecticides and antiplasmodial medicines, but the emergence and spreading of resistant mosquitos and parasites put the efficacy of these interventions at risk¹. In this scenario, an efficient malaria vaccine could be an important additional tool to control and eventually eliminate malaria.

Since the 60's, it has been known that multiple immunizations using irradiated sporozoites can elicit sterile protection against malaria infection. However, during the last 50 years only a few protective antigens were identified, but none of them, individually or in combination, could match the robust protection induced by irradiated parasites.

The most advanced malaria vaccine, RTS,S (Mosquirix, GSK), targets the Plasmodium falciparum circumsporozoite protein (CSP), the major surface protein of sporozoites, the motile stage inoculated in the skin during an infective mosquito bite.

This subunit vaccine reduced the clinical cases of malaria in African infants and children by 26-36%². This partial protection is mainly associated with high titers of anti-CSP antibodies³, and albeit significant, it is far from achieving the standards established by the WHO malaria vaccine road map, which preconizes the development of a vaccine with at least 75% of efficacy against clinical malaria, and ideally targeting morbidity, mortality and parasite transmission⁴.

On the other hand, live irradiated sporozoites can invade but are arrested as early liver-stages inside hepatocytes, conferring sterile immunity against a homologous sporozoite challenge⁵. Unfortunately, technical and economical impediments associated with the production, storage and delivery of these live parasites still hinder their use for mass vaccination in poor tropical countries. This sterile protection seems to be mainly dependent on CD8+ T cells, since their depletion abolishes sterile immunity in several experimental models, however, the identity of the antigens conferring such robust protection is still elusive⁶. So far, the number of known protective antigens among the thousands of possible proteins expressed by pre-erythrocytic (PE) stages, sporozoite and the ensuing liver-stage, is extremely limited, and these antigens only confer weaker protection than CSP alone or in multi-antigenic formulations in humans⁷. To date the attempts to identify new protective antigens from live attenuated sporozoites have not yielded suitable candidates, despite the screening of thousands of PE peptides, mini-genes and genes⁸⁻¹⁰.

Despite these considerable efforts and despite significant improvement in the treatment of the infection and its outcomes, long-term protection on a large scale has not yet been successfully achieved with vaccine candidates. Being challenging in many aspects Malaria thus appears a good candidate to assess the relevancy of a new proposed method to identity protective antigens either as such or in multi-antigen combinations.

In this regard, the invention relates to a method of functionally screening immunogenic and protective properties of antigenic polypeptides, also regarded as screening protective antigenic polypeptides, of a determined pathogen or cancer cells comprising the steps of:
a. optionally pre-selecting candidate antigenic polypeptides for the screening by reference to a group of genes, transcripts or proteins identified for a determined pathogen or cancer cell wherein the antigenic polypeptides may constitute putative targets for a humoral and/or cellular immune responses.
b. providing a lentiviral vector, in particular a HIV-1 based vector, expressing one or more antigenic polypeptide(s) to be assayed for its (their) protective properties,
c. immunizing a non-human mammal in particular a non-human mammal model selected for its susceptibility to infection by the determined pathogen or susceptibility to cancer development, with the lentiviral vector of step b. in immunization dose conditions enabling elicitation of a potent cellular and/or humoral memory response against the antigenic polypeptide(s),
d. challenging the immunized non-human mammal of step c. with the pathogen or administering cancer cells to the immunized non-human mammal of step c. and quantifying the development of the pathogen or the cancer cell in the non-human mammal thereby enabling functional identification of the protective response capacity of the antigenic polypeptide(s).

In a particular embodiment of this method the pathogen is selected in the group of extracellular or intracellular viruses, bacteria, fungi, protozoans and worms.

In particular, the pathogen is selected among arboviruses, hemorrhagic-fever causing viruses, immunodeficiency-causing virus, Mycobacterium spp, Yersinia spp, Listeria ssp, Neisseria spp, Shigella spp, Histoplasma spp, Cryptococcus spp, kinetoplastida parasites, apicomplexan parasites, including malaria parasites, and cestode, trematode or nematode worms.

When a pathogen is cited in general terms in the present disclosure, it also refers especially to a pathogen of the above lists as a particular embodiment.

Alternatively, a method of the invention is suitable for use with antigenic polypeptide(s) which are cancer cell antigens, in particular antigens selected in the group of melanoma, breast carcinoma, B cell lymphoma, colon cancer in particular colon carcinoma, liver cancer, lung cancer, bladder cancer in particular bladder carcinoma, mastocytoma, pancreas cancer and prostate adenocarcinoma. When reference is made in the present disclosure to a cancer cell antigen, it should be read as pertaining also to any of the above listed examples as a particular embodiment.

As example of non-human mammal, in particular animal model suitable for testing according to the method of the invention, rodents are provided, and in particular the following animal are disclosed, which are available to the person skilled in the art: for melanoma: B16F10 C57BL/6 mice (ATCC CRL6745), for breast carcinoma: 4T1 BALB/c mice (ATCC CRL2539), for B cell lymphoma: A20 Balb/C mice (ATCC TIB-208), for bladder cancer: MBT2 C3H/HeN mice (JCRB cell bank IFO50041), for bladder carcinoma: AY27 Fischer rats, for colon cancer: ProB BD9 rats, for colon carcinoma: CT26 BALB/c mice (ATCC CRL2638), for liver cancer: Hepa1-6 C57L mice (ATCC CRL1830), for lung cancer: LL/2 C57BL mice (ATCC CRL1642) or TC1 C57BL/6 mice (ATCC CRL2785), for mastocytoma: P815 DBA/2 mice (ATCC TIB-64), for pancreas cancer: PAN02 C57BL/6 mice, for prostate Adenocarcinoma: R3327H Cop Rat.

The method of the invention may be carried out having recourse to non-pre-selected candidate antigenic polypeptides or, alternatively or in a subsequent step, may be carried out using down-selected antigens as illustrated hereafter for malaria.

By the expression “antigenic polypeptide”, it is intended according to the present invention a polypeptide which may be an antigen of a pathogen or of a cancer cell, in particular a native antigen of a Plasmodium parasite, or expression product of a gene of a Plasmodium parasite, in particular of P. berghei, P. cynomolgi or of a Plasmodium parasite infecting humans such as P. falciparum, P. vivax, P. malariae, P. ovale or P. knowlesi. The application also relates to modified version of such antigenic polypeptides designated as “polypeptidic derivative thereof” which can be a fragment of the native antigen of the pathogen or of the cancer cell, in particular of Plasmodium parasite and especially may be a truncated version of such native antigen.

A derivative polypeptide has an amino acid sequence which is sufficient to provide one or several epitope(s) and which essentially keeps the immunogenic properties of the antigenic polypeptide. It may accordingly have a length of at least about 4 amino acid residues for B epitopes or at least about 8 amino acid residues for T epitopes. In a particular embodiment, the recombinant polynucleotide of the lentiviral vector encodes a fragment of an antigen of the pathogen or of the cancer cell, in particular of the malaria parasite, especially a fragment which results from the deletion of contiguous amino acid residues of the full-length (i.e., native) antigen, provided it keeps the capacity of the native antigen to elicit an immune response in a host.

The polypeptidic derivative as defined hereabove should be considered an alternative to the recited antigenic polypeptide in any definitions or embodiments of the invention unless it appears irrelevant in the context of the disclosure.

The screening method of the invention is in particular intended for the identification of antigenic polypeptides or polypeptidic derivatives thereof which bear T and/or B epitopes as both cellular and humoral immune response may be involved in the protection against the pathogen or the cancer cell, in particular against the malaria parasite, or against the condition or disease resulting from the infection.

The expressions “T-epitope” and “B-epitope” refer to antigenic determinants that are involved respectively in the adaptive immune response driven by T cells and in the immune response driven by B cells. In particular said T-epitopes and respectively B-epitopes elicit T cell, respectively B cell immune response when delivered to the host in suitable conditions. According to a particular embodiment the antigenic polypeptides targeted according to the invention and the polypeptide derivatives of these antigenic polypeptides comprise epitope(s) mediating CD8⁺ T cell response. In a particular embodiment, alternatively or cumulatively, the antigenic polypeptides of the invention and the polypeptide derivatives of these antigenic polypeptides comprise epitope(s) mediating an antibody response.

A particular aspect of the screening according to the invention relates to the use in the designed method of means consisting in lentiviral vectors, in particular HIV-1 based vectors, that show capacity to deliver genes encoding polypeptides to the cells of the non-human mammal model and to elicit a protective immune response when the antigenic polypeptide has such capability.

Accordingly, the method of the invention comprises immunization with one or more than one dose(s) of the assayed antigenic polypeptide.

The term “protective” in the context of the present invention refers to the capability of the screened and selected antigens to elicit a response in the administered host reflected in any of the following outcomes (i) prevention of infection by the pathogen and in particular the Plasmodium parasite, (ii) elimination of the pathogen, in particular Plasmodium parasite and preferably prevention of reintroduction (iii) prevention of the transmission from the infected host to other hosts, in particular prevention of the transmission from a human host infected by a Plasmodium parasite to mosquitoes and preferably prevention of parasite development in mosquitoes, (iv) prevention of clinical symptoms in at least part of the targeted hosts, in particular prevention of a clinical malaria episode in at least part of the hosts infected with Plasmodium parasites. When cancer cells are the target of the response, protection encompasses (i) prevention of the onset of a tumor (ii) prevention of the growth of a tumor, (iii) prevention of vascularization of a tumor. Protection may be high or modest depending on the condition and pathogen and is generally regarded as any benefit for the patient that may impact life-threatening outcomes induced by the pathogen or the cancer cells.

In a particular embodiment that proved to allow the recovery of an increased number of protective polypeptides, the screening method encompasses administering two doses of the lentiviral vector expressing the antigenic polypeptide. Accordingly, the immunization comprises administering one dose of lentiviral vector in a priming step and one dose of lentiviral vector in a boosting step wherein in the priming and boosting steps the lentviral vectors are pseudotyped with different non cross-seroneutralizing envelope proteins and wherein the priming and the boosting doses are different, in particular the boosting dose is higher than the priming dose.

In a particular embodiment of the prime-boost regimen for the immunization, the method of screening encompasses using a dose for boosting which is higher than the dose for priming.

In a further embodiment of the screening method, the administered doses of lentiviral vector expressing the antigenic polypeptide for immunization of the non-human mammal are each in the range of 1×10⁵ to 1×10⁸ TU, in particular 1×10⁵ to 1×10⁷. The inventors have shown that the lentiviral vector may be formulated as a suspension of either concentrated or non-concentrated lentiviral vector without significantly affecting the level of the immune response.

The invention accordingly relates in particular to a method of screening wherein the non-human mammal is immunized successively with at least:

(i) lentiviral vector particles as disclosed herein which are pseudotyped with a first VSV-G envelope of the New Jersey strain;

(ii) provided separately in time from lentiviral vector particles in (i), lentiviral vector particles as disclosed herein which are pseudotyped with a different VSV-G envelope protein wherein said second envelope is obtained from a VSV of the Indiana strain.

The above disclosed embodiments relating to the conditions for priming and boosting the non-human animals apply especially to this embodiment.

The screening method of the invention is defined in a particular embodiment for use with Plasmodium antigens and accordingly encompasses the following features.

To identify critical protective antigens, the invention provides a lentiviral-based immunization screen designed to select plasmodial-conserved antigens capable of protecting susceptible mice against a stringent sporozoite challenge. Using this functional screen the inventors identified 8 protective antigens, including the known vaccine candidates CSP and TRAP, out of 55 tested antigens. Notably, the inventors showed that a combination of 7 antigens sterile protected more than 85% (18/21) of challenged animals versus 5% (1/20) in the CSP immunized group. These findings confirm the relevance of the designed screening test and accordingly pave the way for the development of a multi-antigenic, second-generation pre-erythrocytic malaria vaccine.

The invention thus relates to a method of functionally screening immunogenic properties of antigenic polypeptides of a determined Plasmodium parasite comprising the steps of:
a. optionally pre-selecting candidate antigenic polypeptides for the screening by reference to a group of genes or transcripts identified for a determined Plasmodium parasite wherein the group may reflect a particular stage of the development of the parasite or a particular biological function.
b. providing a lentiviral vector, in particular a HIV-1 based vector, expressing one or more antigenic polypeptide(s) to be assayed for its immunogenic properties,
c. immunizing a non-human mammal selected for its susceptibility to infection by the determined Plasmodium parasite with the lentiviral vector of step b. in immunization dose conditions enabling elicitation of a potent cellular and/or humoral memory response against the antigenic polypeptide,
d. challenging the immunized non-human mammal of step c. with sporozoites of the Plasmodium parasite and quantifying the development of the Plasmodium parasite in the non-human mammal thereby enabling functional identification of the protective response capacity of the antigenic polypeptides.

In a particular embodiment of the invention, the assayed antigenic polypeptide is not a native polypeptide of the parasite but a polypeptide derivative thereof.

The expressions “malaria parasite” and “Plasmodium parasite» are used interchangeably in the present application. They designate every and all forms of the parasite that are associated with the various stages of the parasite cycle in the mammalian, especially human host, including in particular sporozoites, especially sporozoites inoculated in the host skin and present in the blood flow after inoculation, or sporozoites developing in the hepatocytes (liver-stages), merozoites, including especially merozoites produced in the hepatocytes and in the red-blood cells or merozoites developing inside red blood cells (blood-stages). These various forms of the parasite are characterized by multiple specific antigens many of which are well known and identified in the art and some of which are still unknown and to which no biological function has yet been assigned. The antigens can often be designated or classified in groups by reference to their expression according to the stage of the infection. Plasmodium parasites according to the present disclosure encompass parasites infecting human hosts and parasites infecting non-human animals especially rodents and in particular mice. Accordingly, Plasmodium falciparum, Plasmodium vivax, Plasmodium yoelii and Plasmodium berghei are particular examples of these parasites. Plasmodium cynomolgi is another example which is infectious for macaques, and occasionally can infect humans. Antigens of Plasmodium which may be screened according to the method of the invention encompass CSP, TRAP, Celtos, SPECT, ICP, and Facilysin/Bergheilysin or antigens with unknown biological functions such as Ag11-09 and Ag11-10 as disclosed herein. More generally, the method of the invention may be applied to the screening of antigens expressed on gametes, zygotes and ookinetes, antigens of liver-stages or antigens of the asexual blood-stages, as well as, antigens of sporozoites. Accordingly antigens that may be screened include also in particular an antigen selected among P48/P45 antigen and Pfs25.

In a particular embodiment of the invention the method of screening encompasses using antigenic polypeptides characteristic of P. berghei, or P. yoelii and a non-human mammal which is a rodent. It is in particular a mouse susceptible to said Plasmodium parasite, especially a C57BL/6 mouse for P. berghei, or a BALB/c mouse for P. yoelii. According to the invention, the challenging step of the non-human mammal to detect the capacity of the antigenic polypeptide to elicit a protective immune response may be performed by administration to said non-human mammal of the pathogen or the cancer cell antigen providing the antigenic polypeptide, in particular administration of Plasmodium sporozoites, expressing a bioluminescent or a fluorescent marker.

As an example administration of 5,000 to 10,000 Plasmodium sporozoites, 30 days after the last immunization step with the lentiviral vector is suitable for such challenge step.

The time period between the immunization step and the challenge may vary slightly depending on the animals, immunization conditions and assessed pathogen or cancer cell.

Use of a bioluminescent marker is illustrated in the Examples for the Plasmodium parasite and enables in vivo imaging of the load of parasite especially in the liver of the animal, thereby providing indirect detection of the protective immune response elicited by the lentiviral vector bearing the antigenic polypeptide. Bioluminescence detection is for example obtained when the pathogen or the cancer cell, for example the malaria parasite, used for the challenge of the immunized non-human animals, express a constitutive bioluminescent marker, such as luciferase, in particular luciferase-expressing Plasmodium sporozoites. Bioluminescence may be detected by imaging an organ, such as the liver for animal challenged with Plasmodium sporozoites, of the tested non-human animals and quantification of the pathogen or cancer cell cells, in particular quantification of the malaria parasite may be derived therefrom.

Alternatively constitutive fluorescence may be detected when fluorescent pathogens or cancer cells (e.g. expressing GFP), in particular fluorescent Plasmodium parasites, such as GFP-expressing sporozoites, are used for challenging the immunized animals.

In such a case fluorescence may be detected in biological fluids obtained from the animal or in sub-fractions thereof, such as red blood cells harvested from a blood sample previously obtained from the animal. In such a case detection may be carried out by flow cytometry and fluorescence.

Protective immunization in the non-human mammals obtained using the lentiviral vector is assayed by comparing the average pathogen load or tumor growth, in particular the parasite load in the liver, a few days, such as two days after challenge using bioluminescent pathogen or cancer cell, in particular with Plasmodium sporozoites, between the GFP-immunized control group and the test group. The challenge is delivered four weeks following the last immunization step.

When the effect of the lentiviral vector immunization is assessed using fluorescence, protective immunization in the non-human mammal is evaluated by comparing the average pathogen contents or cancer cells contents, in particular the average of log parasitemia with Plasmodium parasite, after a determined duration following challenge, such as at day five post challenge of the GFP-immunized control group and the test group. Alternatively, when development of Plasmodium parasite is assessed, individuals with a log parasitemia inferior to the average of log parasitemia of GFP-group minus 2.5 standard deviations are considered protected. When parasites are not detected in red blood cells after 10 days following the challenge, the immunized animals are considered sterile protected. The challenge is delivered four weeks after the last immunization step.

In a particular embodiment of the invention, the method of screening encompasses the use of a positive control for protection capability against the pathogen or the cancer cell or tumor growth, in particular against infection by Plasmodium parasite or against the parasite-induced condition or disease wherein the positive control is a particular antigen of the pathogen or the cancer cell, such as CSP (circumsporozoite protein) or TRAP for Plasmodium parasite, and using a negative control for protective capability, which is GFP or a parasite antigen known to be non protective. In a particular embodiment of the invention, the method of screening encompasses a step wherein immune-sera in the sample obtained from an immunized non-human mammal are incubated with GFP-expressing Plasmodium sporozoites wherein a subgroup of these sporozoites is permeabilized and a subgroup of these sporozoites is not permeabilized. Such a step allows to identify the location of the target of immune reaction elicited by the antigen expressed by the lentiviral particles.

In a particular embodiment of the invention, the lentiviral vector used in the screening contains in its genome a synthetic gene for an antigenic polypeptide of the pathogen or the cancer cells, such as an antigenic polypeptide of Plasmodium, the coding sequence of which is codon-optimized for the expression of the antigenic polypeptide in mammalian cells and wherein expression of the antigenic polypeptide is driven by a promoter suitable for directing gene expression in various mammalian cell types, including dendritic cells, such as a beta-2 microglobulin promoter. The presence of the codon-optimized gene enables a more efficient expression of the antigenic polypeptide in mammalian cells.

In a further embodiment of the invention, the synthetic codon-optimized gene sequence is cloned into restriction sites of a pTRIP plasmid harboring a determined promoter, such pTRIP plasmid providing the transfer vector for the preparation of the genome of the vector particles. This plasmid is hence used for the production of the lentiviral particles when it is co-transfected with other plasmids expressing on the one hand the packaging construct for the particles and on the other hand the envelope of the particles. Plasmid pTRIP has been disclosed in the art, especially in the cited patent applications disclosing lentiviral vector preparation. In addition to the choice of the promoter driving the expression of the antigenic polypeptide, the transfer vector may comprise regulatory elements that increase the expression of the antigenic polypeptides. Such regulatory elements may be a WPRE sequence (woodchuck hepatitis virus).

In a particular embodiment of the invention, the codon-optimized sequence is a mammal-codon optimized sequence, in particular a rodent- or a mouse-codon optimized sequence.

As disclosed herein the screening method of the invention may encompass a pre-selection of the candidate antigenic polypeptides to be assayed for their capacity to elicit a protective immune response. Such pre-selection step may include a step of transcriptome and proteome profiling of the pathogen or the cancer cell, or a step of comparative transcriptome and proteome profiling at different stages of pathogen or cancer development if this appears to be relevant.

In particular when the malaria parasite is concerned, a step of transcriptome and proteome profiling for of the pre-erythrocytic stages of development is performed and compared with the transcriptome and proteome profiling of the parasite in another stage of development, in particular in the blood-stage of development of said parasite, to identify overexpressed transcripts in the pre-erythrocytic stages of the parasite. Overexpressed transcripts of the pre-erythrocytic stages are considered to be preferred candidates for screening with a view to identify vaccine candidates using antigenic polypeptides harboring ability to elicit a protective immune response in a host.

When an antigenic polypeptide of a determined Plasmodium species is identified as a candidate for the screening, the orthologous gene in a Plasmodium parasite infecting a human host, in particular Plasmodium falciparum, Plasmodium vivax is determined. The detailed disclosure provided in respect of screening of antigens of Plasmodium may be transposed to other pathogens. In such a case reporter genes suitable for use with the particular pathogens may have to be selected. The following table illustrates suitable combinations of various pathogens and reporter genes.

| | pathogen | reporter gene | reference |
|---|---|---|---|
| parasites | L. major | luciferase | Mears et al., PLoS pathogens 2015 (i) |
| | L. amazonensis | luciferase | Mears et al., PLoS pathogens 2015 |
| | L. major | eGFP | Mears et al., PLoS pathogens 2015 |
| | L. panamensis | eGFP | Mears et al., PLoS pathogens 2015 |
| | L. major | mCherry | Mears et al., PLoS pathogens 2015 |
| | L. major | fusion eGFP- | Mears et al., PLoS pathogens 2015 |
| | | luciferase | |
| | Entomeaba histolytica | luciferase | Andreu et al., FEMS Microbiol Rev. 2011 (ii) |
| | Schistosoma mansoni | wt pathogen* | Krautz-Peterson et al., FASEB J 2009 (iii) |
| | Toxoplasma gondii | luciferase | Andreu et al., FEMS Microbiol Rev. 2011 |
| | Trypanosoma brucei | luciferase | Andreu et al., FEMS Microbiol Rev. 2011 |
| | Trypanosoma cruzi | luciferase | Andreu et al., FEMS Microbiol Rev. 2011 |
| viruses | influenza virus | luciferase | Tran et al., JVI 2013 (iv) |
| | influenza virus | ‘Color-flu’ | Fukuyama et al, Nature Com 2015 (v) |
| | | viruses** | |
| | Herpes simplex virus | luciferase | Andreu et al., FEMS Microbial Rev. 2011 |
| | Type I | | |
| | Varicella zoster virus | luciferase | Andreu et al., FEMS Microbial Rev. 2011 |
| bacteria | Bacillus anthracis | luciferase | Andreu et al., FEMS Microbial Rev. 2011 |
| | Brucella melitensis | luciferase | Andreu et al., FEMS Microbial Rev. 2011 |
| | Escherichia coli | luciferase | Andreu et al., FEMS Microbial Rev. 2011 |
| | Haemophilus influenza | luciferase | Andreu et al., FEMS Microbial Rev. 2011 |
| | Listeria | luciferase | Andreu et al., FEMS Microbial Rev. 2011 |
| | monocytogenes | | |
| | Mycobacterium | luciferase | Andreu et al., FEMS Microbial Rev. 2011 |
| | tuberculosis | | |
| | Mycobacterium | wt pathogen*** | Kong et al., PNAS 2010 (vi) |
| | tuberculosis | | |
| | Pseudomonas | luciferase | Andreu et al., FEMS Microbial Rev. 2011 |
| | aeruginosa | | |
| | Salmonella enterica | luciferase | Andreu et al., FEMS Microbial Rev. 2011 |
| | Typhimurium | | |
| | Staphylococcus | luciferase | Andreu et al., FEMS Microbial Rev. 2011 |
| | aureus | | |
| | Streptococcus | luciferase | Andreu et al., FEMS Microbial Rev. 2011 |
| | pneumonia | | |
| | Yersinia enterocolitica | luciferase | Andreu et al., FEMS Microbial Rev. 2011 |
| fungi | Aspergillus fumigatus | luciferase | Andreu et al., FEMS Microbial Rev. 2011 |
| | Candida albicans | luciferase | Andreu et al., FEMS Microbial Rev. 2011 |

*The pathogen is not recombinant for a reporter gene, NIR imaging agent ProSense 680 and fluorescence molecular tomography (FMT) imaging are used. The near-infrared imaging agent ProSense 680 was injected into the vasculature of mice that were exposed to infectious schistosome larvae (cercariae). The feeding schistosomes ingest the intravenous injected imaging agent as part of their blood meal and their abundant intestinal cathepsins cleave the compound to release measurable fluorochrome in the parasite's gut.
**several fluorescent proteins of different colours
***The pathogen is not recombinant for a reporter gene, a near-infrared fluorogenic substrate which is eukaryotic cell permeable and activated by β-lactamase-producing bacteria is used.
(i) http://journals.plos.org/plosntds/article?id=10.1371/journal.pntd.0003889
(ii) https://www.ncbi.nlm.nih.gov/pmc/articles/PMC3084502/#b183
(iii) https://www.ncbi.nlm.nih.gov/pmc/articles/PMC2717771/
(iv) https://www.ncbi.nlm.nih.gov/pmc/articles/PMC3838222/
(v) http://www.nature.com/articles/ncomms7600
(vi) https://www.ncbi.nlm.nih.gov/pmc/articles/PMC2901431/

### Detailed Description of the Lentiviral Vectors for Use According to the Invention

In order to carry out the method of screening of the invention lentiviral vector are used which are lentiviral particles (i.e. vector particles), and which may be replication-incompetent lentiviral vectors, especially replication-incompetent HIV-1 based vectors characterized in that (i) they are pseudotyped with a determined heterologous viral envelope protein or viral envelope proteins originating from a RNA virus which is not HIV and (ii) they comprise in their genome at least one recombinant polynucleotide encoding at least one antigenic polypeptide (or polypeptide derivative thereof) carrying epitope(s) of an antigen of a specific group or stage of development of a Plasmodium parasite, in particular antigen which is a pre-erythrocytic stage antigen or a polypeptidic derivative thereof of a Plasmodium parasite capable of infecting a determined non-human mammalian host used for screening, and wherein said epitope(s) encompass(es) T-epitope(s).

In a particular embodiment of the invention, the encoded antigenic polypeptide, in particular of a pre-erythrocytic stage antigen of a Plasmodium parasite further comprises B-epitope(s).

According to a particular embodiment of the invention, the lentiviral vectors are either designed to express proficient (i.e., integrative-competent) or deficient (i.e., integrative-incompetent) particles.

The preparation of the lentiviral vectors is well known from the skilled person and has been extensively disclosed in the literature (confer for review Sakuma T. et al (Biochem. J. (2012) 443, 603-618). The preparation of such vectors is also illustrated herein in the Examples.

In a particular embodiment of the invention, the polynucleotide(s) encoding the antigenic polypeptides of the lentiviral vector has(have) been mammal-codon optimized (CO). Optionally the lentiviral sequences of the genome of said particles have also a mammal-codon optimized nucleotide sequence. In a particular aspect of the invention the codon optimization has been carried out for expression in mouse cells when the screening method is performed in mice.

It has been observed that codon optimized nucleotide sequences, especially when optimized for expression in mammalian cells, enable the production of higher yield of particles in such mammalian cells. Production cells are illustrated in the examples. Accordingly, when lentiviral vector particles of the invention are administered to a mammalian, higher amounts of particles are produced in said host which favour the elicitation of a strong immune response.

The lentiviral vector (or lentiviral vectors particles or lentiviral-based vector particles) defined in the present invention are pseudotyped lentiviral vectors consisting of vector particles bearing envelope protein or envelope proteins which originate from a virus different from the particular lentivirus (especially a virus different from HIV, in particular HIV-1), which provides the vector genome of the lentiviral vector particles. Accordingly, said envelope protein or envelope proteins, are “heterologous” viral envelope protein or viral envelope proteins with respect to the vector genome of the particles. In the following pages, reference will also be made to “envelope protein(s)” to encompass any type of envelope protein or envelope proteins suitable to perform the invention.

When reference is made to “lentiviral” vectors (lentiviral-based vectors) in the application, it relates in particular, to HIV-based vectors and especially HIV-1-based vectors.

The lentiviral vectors suitable to perform the invention are so-called replacement vectors, meaning that the sequences of the original lentivirus encoding the lentiviral proteins are essentially deleted in the genome of the vector or, when present, are modified, and especially mutated, especially truncated, to prevent expression of biologically active lentiviral proteins, in particular, in the case of HIV, to prevent the expression by said transfer vector, of functional ENV, GAG, and POL proteins and optionally of further structural and/or accessory and/or regulatory proteins of the lentivirus, especially of HIV. In a particular embodiment, the lentiviral vector is a first-generation vector, in particular a first-generation of a HIV-based vector which is characterized in that it is obtained using separate plasmids to provide (i) the packaging construct, (ii) the envelope and (iii) the transfer vector genome. Alternatively it may be a second-generation vector, in particular a second-generation of a HIV-based vector which in addition, is devoid of viral accessory proteins (such as in the case of HIV-1, Vif, Vpu, Vpr or Nef) and therefore includes only four out of nine HIV full genes: gag, pol, tat and rev. In another embodiment, the vector is a third-generation vector, in particular a third-generation of a HIV-based vector which is furthermore devoid of said viral accessory proteins and also is Tat-independent; these third-generation vectors may be obtained using 4 plasmids to provide the functional elements of the vector, including one plasmid encoding the Rev protein of HIV when the vector is based on HIV-1. Such vector system comprises only three of the nine genes of HIV-1. The structure and design of such generations of HIV-based vectors is well known in the art. The “vector genome” of the vector particles is a recombinant nucleic acid which also comprises the polynucleotide or transgene of interest encoding the antigenic polypeptide(s) of malaria parasite which are candidates for the screening. The lentiviral-based sequence and polynucleotide/transgene of the vector genome are borne by a plasmid vector thus giving rise to the “transfer vector” also referred to as “sequence vector”. Accordingly, these expressions are used interchangeably in the present application.

The vector genome as defined herein accordingly contains, apart from the so-called recombinant polynucleotide placed under control of proper regulatory sequences for its expression, the sequences of the original lentiviral genome which are non-coding regions of said genome, and are necessary to provide recognition signals for DNA or RNA synthesis and processing (mini-viral genome). These sequences are cis-acting sequences necessary for packaging (Ψ), reverse transcription (LTRs possibly mutated with respect to the original ones) and transcription and optionally integration (RRE) and furthermore for the particular purpose of the invention, they contain a functional sequence favouring nuclear import in cells and accordingly transgene transfer efficiency in said cells, which element is described as a DNA Flap element that contains or consists of the so-called central cPPT-CTS nucleotidic domain present in lentiviral genome sequences especially in HIV-1 or in some retroelements such as those of yeasts.

The structure and composition of the vector genome used to prepare the lentiviral vectors of the invention are based on the principles described in the art and on examples of such lentiviral vectors primarily disclosed in (Zennou et al, 2000; Firat H. et al, 2002; VandenDriessche T. et al). Constructs of this type have been deposited at the CNCM (Institut Pasteur, France) as will be referred to herein. In this respect reference is also made to the disclosure, including to the deposited biological material, in patent applications WO 99/55892, WO 01/27300 and WO 01/27304.

According to a particular embodiment of the invention, a vector genome may be a replacement vector in which all the viral protein coding sequences between the 2 long terminal repeats (LTRs) have been replaced by the recombinant polynucleotide encoding the polypeptide of the malaria parasite, and wherein the DNA-Flap element has been re-inserted in association with the required cis-acting sequences described herein. Further features relating to the composition of the vector genome are disclosed in relation to the preparation of the particles.

In a particular embodiment of the invention one lentiviral vector encodes one antigenic polypeptide of the Plasmodium parasite.

In a particular embodiment, a lentiviral vector of the invention may comprise in its genome one or more than one recombinant polynucleotide encoding at least one antigenic polypeptide carrying epitope(s) of determined group of antigens, such as antigens of a determined development stage of the parasite, in particular a pre-erythrocytic stage antigen, as disclosed herein. In particular, said vector genome comprises two polynucleotides which are consecutive or separated on the genome and which encode different polypeptides of either the same or distinct antigens of the determined group, in particular of the pre-erythrocytic stage of a Plasmodium parasite or different antigenic polypeptides of distinct antigens of different forms of the malaria parasite, especially antigens of the pre-erythrocytic stage and antigens of the erythrocytic stage of the parasite.

In a particular embodiment, the vector genome contains two or more recombinant polynucleotides, each of them encoding a distinct antigenic polypeptide and each polypeptide originating from a different antigen of a determined group, in particular of a determined development stage, including the pre-erythrocytic stage, of the malaria parasite.

The description made herein in respect to antigenic polypeptides similarly applies to polypeptidic derivatives thereof.

Particular features of the lentiviral vectors used in accordance with the various embodiments of the invention are also disclosed in the Examples, such features being either taken alone or in combination to produce the vectors.

According to the invention, the lentiviral vector particles are pseudotyped with a heterologous viral envelope protein or viral polyprotein of envelope originating from a RNA virus which is not the lentivirus providing the lentiviral sequences of the genome of the lentiviral particles.

As examples of typing envelope proteins for the preparation of the lentiviral vector, the invention relates to viral transmembrane glycosylated (so-called G proteins) envelope protein(s) of a Vesicular Stomatitis Virus (VSV), which is(are) for example chosen in the group of VSV-G protein(s) of the Indiana strain and VSV-G protein(s) of the New Jersey strain.

The envelope glycoprotein of the vesicular stomatitis virus (VSV-G) is a transmembrane protein that functions as the surface coat of the wild type viral particles.

It is also a suitable coat protein for engineered lentiviral vectors. Presently, nine virus species are definitively classified in the VSV gender, and nineteen rhabdoviruses are provisionally classified in this gender, all showing various degrees of cross-neutralisation. When sequenced, the protein G genes indicate sequence similarities.

The VSV-G protein presents a N-terminal ectodomain, a transmembrane region and a C-terminal cytoplasmic tail. It is exported to the cell surface via the transGolgi network (endoplasmic reticulum and Golgi apparatus).

Vesicular stomatitis Indiana virus (VSIV) and Vesicular stomatitis New Jersey virus (VSNJV) are preferred strains to pseudotype the lentiviral vectors of the invention, or to design recombinant envelope protein(s) to pseudotype the lentiviral vectors. Their VSV-G proteins are disclosed in GenBank, where several strains are presented. For VSV-G New Jersey strain reference is especially made to the sequence having accession number V01214. For VSV-G of the Indiana strain, reference is made to the sequence having accession number AAA48370.1 in Genbank corresponding to strain JO2428.

Said viral envelope protein(s) are capable of uptake by antigen presenting cells and especially by dendritic cells including by liver dendritic cells by mean of fusion and/or of endocytosis. In a particular embodiment, the efficiency of the uptake may be used as a feature to choose the envelope of a VSV for pseudotyping. In this respect the relative titer of transduction (Titer DC/Titer of other transduced cells e.g. 293T cells) may be considered as a test and envelope having a relative good ability to fuse with DC would be preferred.

Antigen Presenting Cells (APC) and especially Dentritic cells (DC) are proper target cells for pseudotyped lentiviral vectors which are used as immune compositions accordingly.

The VSV-G envelope protein(s) are expressed from a polynucleotide containing the coding sequence for said protein(s), which polynucleotide is inserted in a plasmid (designated envelope expression plasmid or pseudotyping env plasmid) used for the preparation of the lentiviral vector particles of the invention. The polynucleotide encoding the envelope protein(s) is under the control of regulatory sequences for the transcription and/or expression of the coding sequence (including optionally post-transcriptional regulatory elements (PRE), in particular such elements that may improve the antigen expression from the vector, especially a polynucleotide such as the element of the Woodchuck hepatitis virus, i.e. the WPRE sequence, obtainable from Invitrogen).

Accordingly, a nucleic acid construct is provided which comprises an internal promoter suitable for the use in mammalian cells, especially in human cells in vivo and the nucleic acid encoding the envelope protein under the control of said promoter. A plasmid containing this construct is used for transfection or for transduction of cells suitable for the preparation of vector particles. Promoters may in particular be selected for their properties as constitutive promoters, tissue-specific promoters, or inducible promoters. Examples of suitable promoters encompass the promoters of the following genes: MHC class1 promoter, human beta-2 microglobulin gene (β2M promoter), EF1α, human PGK, PPI (preproinsulin), thiodextrin, HLA DR invariant chain (P33), HLA DR alpha chain, Ferritin L chain or Ferritin H chain, Chymosin beta 4, Chymosin beta 10, Cystatin Ribosomal Protein L41, CMVie or chimeric promoters such as GAG (CMV early enhancer/chicken β actin) disclosed in Jones S. et al (Jones S. et al Human Gene Therapy, 20:630-640 (June 2009)).

These promoters may also be used in regulatory expression sequences involved in the expression of gag-pol derived proteins from the encapsidation plasmids, and/or to express the antigenic polypeptides from the transfer vector.

Alternatively, when the envelope expression plasmid is intended for expression in stable packaging cell lines, especially for stable expression as continuously expressed viral particles, the internal promoter to express the envelope protein(s) is advantageously an inducible promoter such as one disclosed in Cockrell A. S. et al. (Mol. Biotechnol. (2007) 36:184-204). As examples of such promoters, reference is made to tetracycline and ecdysone inducible promoters. The packaging cell line may be the STAR packaging cell line (ref Cockrell A. S. et al (2007), Ikedia Y. et al (2003) Nature Biotechnol. 21: 569-572) or a SODk packaging cell line, such as SODk0 derived cell lines, including SODk1 and SODk3 (ref Cockrell A. S. et al (2007), Cockrell A; S. et al (2006) Molecular Therapy, 14: 276-284, Xu K. et al. (2001), Kafri T. et al (1999) Journal of Virol. 73:576-584).

According to the invention, the lentiviral vector are the product recovered from co-transfection of mammalian cells, with:
a vector plasmid comprising (i) lentiviral, especially HIV-1, cis-active sequences necessary for packaging, reverse transcription, and transcription and further comprising a functional lentiviral, especially derived from HIV-1, DNA flap element and (ii) a polynucleotide encoding an antigenic polypeptide of a malaria parasite as disclosed herein under the control of regulatory expression sequences, and optionally comprising sequences for integration into the genome of the host cell;
an expression plasmid encoding a pseudotyping envelope derived from a RNA virus, said expression plasmid comprising a polynucleotide encoding an envelope protein or proteins for pseudotyping, wherein said envelope pseudotyping protein is advantageously from a VSV and is in particular a VSV-G of the Indianan strain or of the New Jersey strain and,
an encapsidation plasmid, which either comprises lentiviral, especially HIV-1, gag-pol packaging sequences suitable for the production of integration-competent vector particles or modified gag-pol packaging sequences suitable for the production of integration-deficient vector particles.

The invention thus also concerns lentiviral vector particles as described above, which are the product recovered from a stable cell line transfected with:
a vector plasmid comprising (i) lentiviral, especially HIV-1, cis-active sequences necessary for packaging, reverse transcription, and transcription and further comprising a functional lentiviral, especially HIV-1, DNA flap element and optionally comprising cis-active sequences necessary for integration, said vector plasmid further comprising (ii) a polynucleotide of a codon-optimized sequence for a determined non-human mammal of the gene encoding the antigenic polypeptide of a Plasmodium parasite, under the control of regulatory expression sequences, especially a promoter;
a VSV-G envelope expression plasmid comprising a polynucleotide encoding a VSV-G envelope protein in particular VSV-G of the Indiana strain or of the New Jersey strain, wherein said polynucleotide is under the control of regulating expression sequences, in particular regulatory expression sequences comprising an inducible promoter, and;
an encapsidation plasmid, wherein the encapsidation plasmid either comprises lentiviral, especially HIV-1, gag-pol coding sequences suitable for the production of integration-competent vector particles or modified gag-pol coding sequences suitable for the production of integration-deficient vector particles, wherein said gag-pol sequences are from the same lentivirus sub-family as the DNA flap element, wherein said lentiviral gag-pol or modified gag-pol sequence is under the control of regulating expression sequences.

The stable cell lines expressing the vector particles of the invention are in particular obtained by transduction of the plasmids.

The polynucleotide encodes at least one antigenic polypeptide of a malaria parasite according to any embodiment disclosed in the present specification. In particular, it encodes a polypeptide which is a truncated mammalian, especially human, codon-optimized sequence coding for such antigenic polypeptide of Plasmodium falciparum, Plasmodium vivax, Plasmodium malariae, Plasmodium ovale, Plasmodium knowlesi or Plasmodium berghei.

In a particular embodiment, the polynucleotide encodes two or more antigenic polypeptides of the malaria parasite which originate and/or are derived from distinct antigens of said parasite as disclosed in the various embodiments. Accordingly, the vector plasmid may comprise several expression cassettes for the expression of the various antigenic polypeptides or may comprise bicistronic or multicistronic expression cassettes where the polynucleotides encoding the various polypeptides are separated by an IRES sequence of viral origin (Internal Ribosome Entry Site), or it may encode fusion protein(s).

The internal promoter contained the vector genome and controlling the expression of the polynucleotide encoding an antigenic polypeptide of the malaria parasite (as a transgene or in an expression cassette) may be selected from the promoters of the following genes: human beta-2 microglobulin gene (β2M promoter), EF1α, human PGK, PPI (preproinsulin), thiodextrin, HLA DR invariant chain (P33), HLA DR alpha chain, Ferritin L chain or Ferritin H chain, Chymosin beta 4, Chymosin beta 10, or Cystatin Ribosomal Protein L41 CMVie or chimeric promoters such as GAG(CMV early enhancer/chicken β actin) disclosed in Jones S. et al (2009).

A promoter among the above cited internal promoters may also be selected for the expression of the envelope protein(s) and packaging (gag-pol derived) proteins.

Alternatively, vector particles can be produced from co-transfection of the plasmids disclosed herein, in stable packaging cell lines which thus become capable of continuously secreting vector particles. Promoters used in the regulatory expression sequences involved for the expression of the envelope protein(s) are advantageously inducible promoters.

The following particular embodiments may be carried out when preparing the lentiviral vector based on human lentivirus, and especially based on HIV-1 virus.

According to the invention, the genome of the lentiviral vector is derived from a human lentivirus, especially from the HIV lentivirus. In particular, the pseudotyped lentiviral vector is an HIV-based vector, such as an HIV-1, or HIV-2 based vector, in particular is derived from HIV-1M, for example from the BRU or LAI isolates. Alternatively, the lentiviral vector providing the necessary sequences for the vector genome may be originating from lentiviruses such as EIAV, CAEV, VISNA, FIV, BIV, SIV, HIV-2, HIV-O which are capable of transducing mammalian cells.

As stated above, when considering it apart from the recombinant polynucleotide that it finally contains, the vector genome is a replacement vector in which the nucleic acid between the 2 long terminal repeats (LTRs) in the original lentivirus genome have been restricted to cis-acting sequences for DNA or RNA synthesis and processing, including for the efficient delivery of the transgene to the nuclear of cells in the host, or at least are deleted or mutated for essential nucleic acid segments that would enable the expression of lentiviral structure proteins including biological functional GAG polyprotein and possibly POL and ENV proteins.

In a particular embodiment, the 5′ LTR and 3′ LTR sequences of the lentivirus are used in the vector genome, but the 3′-LTR at least is modified with respect to the 3′LTR of the original lentivirus at least in the U3 region which for example can be deleted or partially deleted for the enhancer. The 5′LTR may also be modified, especially in its promoter region where for example a Tat-independent promoter may be substituted for the U3 endogenous promoter.

In a particular embodiment the vector genome comprises one or several of the coding sequences for Vif-, Vpr, Vpu- and Nef-accessory genes (for HIV-1 lentiviral vectors). Alternatively, these sequences can be deleted independently or each other or can be non-functional (second-generation lentiviral vector).

The vector genome of the lentiviral vector particles comprises, as an inserted cis-acting fragment, at least one polynucleotide consisting in the DNA flap element or containing such DNA flap element. In a particular embodiment, the DNA flap is inserted upstream of the polynucleotide encoding the antigenic polypeptide of Plasmodium parasite, and is advantageously—although not necessarily—located in an approximate central position in the vector genome. A DNA flap suitable for the invention may be obtained from a lentivirus, in particular a human lentivirus especially a HIV-1 retrovirus, or from a retrovirus-like organism such as retrotransposon. It may be alternatively obtained from the CAEV (Caprine Arthritis Encephalitis Virus) virus, the EIAV (Equine Infectious Anaemia Virus) virus, the VISNA virus, the SIV (Simian Immunodeficiency Virus) virus or the FIV (Feline Immunodeficiency Virus) virus. The DNA flap may be either prepared synthetically (chemical synthesis) or by amplification of the DNA providing the DNA Flap from the appropriate source as defined above such as by Polymerase chain reaction (PCR). In a more preferred embodiment, the DNA flap is obtained from an HIV retrovirus, for example HIV-1 or HIV-2 virus including any isolate of these two types.

The DNA flap (also designated cPPT/CTS) (defined in Zennou V. et al. ref 27, 2000, Cell vol 101, 173-185 or in WO 99/55892 and WO 01/27304), is a structure which is central in the genome of some lentiviruses especially in HIV, where it gives rise to a 3-stranded DNA structure normally synthesized during especially HIV reverse transcription and which acts as a cis-determinant of HIV genome nuclear import. The DNA flap enables a central strand displacement event controlled in cis by the central polypurine tract (cPPT) and the central termination sequence (CTS) during reverse transcription. When inserted in lentiviral-derived vectors, the polynucleotide enabling the DNA flap to be produced during reverse-transcription, stimulates gene transfer efficiency and complements the level of nuclear import to wild-type levels (Zennou et al., Cell, 2000).

Sequences of DNA flaps have been disclosed in the prior art, especially in the above cited patent applications. These sequences are also disclosed in the sequence of SEQ ID No. 1 from position 2056 to position 2179. They are preferably inserted as a fragment, optionally with additional flanking sequences, in the vector genome, in a position which is preferably near the centre of said vector genome. Alternatively they may be inserted immediately upstream from the promoter controlling the expression of the polynucleotide(s) encoding the antigenic polypeptide. Said fragments comprising the DNA flap, inserted in the vector genome may have a sequence of about 80 to about 200 bp, depending on its origin and preparation.

According to a particular embodiment, a DNA flap has a nucleotide sequence of about 90 to about 140 nucleotides.

In HIV-1, the DNA flap is a stable 99-nucleotide-long plus strand overlap. When used in the genome vector of the lentiviral vector of the invention, it may be inserted as a longer sequence, especially when it is prepared as a PCR fragment. A particular appropriate polynucleotide comprising the structure providing the DNA flap is a 124-base pair polymerase chain reaction (PCR) fragment encompassing the cPPT and CTS regions of the HIV-1 DNA (as disclosed in SEQ ID No. 1)

It is specified that the DNA flap used in the genome vector and the polynucleotides of the encapsidation plasmid encoding the GAG and POL polyproteins should originate from the same lentivirus sub-family or from the same retrovirus-like organism.

Preferably, the other cis-activating sequences of the genome vector also originate from the same lentivirus or retrovirus-like organism, as the one providing the DNA flap.

The vector genome may further comprise one or several unique restriction site(s) for cloning the recombinant polynucleotide.

In a preferred embodiment, in said vector genome, the 3′ LTR sequence of the lentiviral vector genome is devoid of at least the activator (enhancer) and possibly the promoter of the U3 region. In another particular embodiment, the 3′ LTR region is devoid of the U3 region (delta U3). In this respect, reference is made to the description in WO 01/27300 and WO 01/27304.

In a particular embodiment, in the vector genome, the U3 region of the LTR 5′ is replaced by a non lentiviral U3 or by a promoter suitable to drive tat-independent primary transcription. In such a case, the vector is independent of tat transactivator (third generation vector).

The vector genome also comprises the psi (w) packaging signal. The packaging signal is derived from the N-terminal fragment of the gag ORF. In a particular embodiment, its sequence could be modified by frameshift mutation(s) in order to prevent any interference of a possible transcription/translation of gag peptide, with that of the transgene.

The vector genome may optionally also comprise elements selected among a splice donor site (SD), a splice acceptor site (SA) and/or a Rev-responsive element (RRE). According to a particular embodiment, the vector plasmid (or added genome vector) comprises the following cis-acting sequences for a transgenic expression cassette:
1. The LTR sequence (Long-Terminal Repeat), required for reverse transcription, the sequences required for transcription and including optionally sequences for viral DNA integration. The 3′ LTR is deleted in the U3 region at least for the promoter to provide SIN vectors (Self-inactivating), without perturbing the functions necessary for gene transfer, for two major reasons: first, to avoid trans-activation of a host gene, once the DNA is integrated in the genome and secondly to allow self-inactivation of the viral cis-sequences after retrotranscription. Optionally, the tat-dependent U3 sequence from the 5′-LTR which drives transcription of the genome is replaced by a non endogenous promoter sequence. Thus, in target cells only sequences from the internal promoter will be transcribed (transgene).
2. The p region, necessary for viral RNA encapsidation.
3. The RRE sequence (REV Responsive Element) allowing export of viral messenger RNA from the nucleus to the cytosol after binding of the Rev protein.
4. The DNA flap element (cPPT/CTS) to facilitate nuclear import.
5. Optionally post-transcriptional elements such as the WPRE cis-active sequence (Woodchuck hepatitis B virus Post-Responsive Element) also added to optimize stability of mRNA (Zufferey et al., 1999), the matrix or scaffold attachment regions (SAR and MAR sequences) such as those of the immunoglobulin-kappa gene (Park F. et al Mol Ther 2001; 4: 164-173).

The lentiviral vector of the invention is non replicative (replication-incompetent) i.e., the vector and lentiviral vector genome are regarded as suitable to alleviate concerns regarding replication competent lentiviruses and especially are not able to form new particles budding from the infected host cell after administration. This may be achieved in well known ways as the result of the absence in the lentiviral genome of the gag, pol or env genes, or their absence as “functional genes”. The gag and pol genes are thus, only provided in trans. This can also be achieved by deleting other viral coding sequence(s) and/or cis-acting genetic elements needed for particles formation.

By “functional” it is meant a gene that is correctly transcribed, and/or correctly expressed. Thus, if present in the lentiviral vector genome of the invention in this embodiment contains sequences of the gag, pol, or env are individually either not transcribed or incompletely transcribed; the expression “incompletely transcribed” refers to the alteration in the transcripts gag, gag-pro or gag-pro-pol, one of these or several of these being not transcribed. Other sequences involved in lentiviral replication may also be mutated in the vector genome, in order to achieve this status. The absence of replication of the lentiviral vector should be distinguished from the replication of the lentiviral genome. Indeed, as described before, the lentiviral genome may contain an origin of replication ensuring the replication of the lentiviral vector genome without ensuring necessarily the replication of the vector particles.

In order to obtain lentiviral vectors according to the invention, the vector genome (as a vector plasmid) must be encapsidated in particles or pseudo-particles. Accordingly, lentiviral proteins, except the envelope proteins, have to be provided in trans to the vector genome in the producing system, especially in producing cells, together with the vector genome, having recourse to at least one encapsidation plasmid carrying the gag gene and either the pol lentiviral gene or an integrative-incompetent pol gene, and preferably lacking some or all of the coding sequences for Vif-, Vpr, Vpu- and Nef-accessory genes and optionally lacking Tat (for HIV-1 lentiviral vectors).

A further plasmid is used, which carries a polynucleotide encoding the envelope pseudotyping protein(s) selected for pseudotyping lentiviral vector particles.

In a preferred embodiment, the packaging plasmid encodes only the lentiviral proteins essential for viral particle synthesis. Accessory genes whose presence in the plasmid could raise safety concerns are accordingly removed. Accordingly, viral proteins brought in trans for packaging are respectively as illustrated for those originating from HIV-1:
1. GAG proteins for building of the matrix (MA, with apparent Molecular Weight p17), the capsid (CA, p24) and nucleocapsid (NC, p6).
2. POL encoded enzymes: integrase, protease and reverse transcriptase.
3. TAT and REV regulatory proteins, when TAT is necessary for the initiation of LTR-mediated transcription; TAT expression may be omitted if the U3 region of 5′LTR is substituted for a promoter driving tat-independent transcription. REV may be modified and accordingly used for example in a recombinant protein which would enable recognition of a domain replacing the RRE sequence in the vector genome, or used as a fragment enabling binding to the RRE sequence through its RBD (RNA Binding Domain).

In order to avoid any packaging of the mRNA generated from the genes contained in the packaging plasmid in the viral particles, the p region is removed from the packaging plasmid. A heterologous promoter is inserted in the plasmid to avoid recombination issues and a poly-A tail is added 3′ from the sequences encoding the proteins.

Appropriate promoters have been disclosed above.

The envelope plasmid encodes the envelope protein(s) for pseudotyping which are disclosed herein, under the control of an internal promoter, as disclosed herein.

Any or all the described plasmids for the preparation of the lentiviral vector particles of the invention may be codon optimized (CO) in the segment encoding proteins. Codon optimization according to the invention is preferably performed to improve translation of the coding sequences contained in the plasmids, in mammalian cells, murine or especially human cells. According to the invention, codon optimization is especially suited to directly or indirectly improve the preparation of the vector particles or to improve their uptake by the cells of the host to whom they are administered, or to improve the efficiency of the transfer of the polynucleotide encoding the antigenic polypeptide of the malaria parasite (transgene) in the genome of the transduced cells of the host. Methods for optimizing codons are well known in the art and codon optimization is especially performed using available programs to that effect. Codon optimization is illustrated for the coding sequences used in the examples.

In a particular embodiment of the invention, the pseudotyped lentiviral vector is also, or alternatively, integrative-incompetent. In such a case, the vector genome and thus the recombinant polynucleotide which it contains do not integrate into the genome of the transduced cells or in the cells of the host to whom it has been administered.

The present invention relates to the use of a lentiviral vector wherein the expressed integrase protein is defective and which further comprises a polynucleotide especially encoding at least one antigenic polypeptide carrying epitope(s) of a pre-erythrocytic stage antigen of a Plasmodium parasite, in an immunogenic composition.

By “integration-incompetent”, it is meant that the integrase, preferably of lentiviral origin, is devoid of the capacity of integration of the lentiviral genome into the genome of the host cells i.e., an integrase protein mutated to specifically alter its integrase activity.

Integration-incompetent lentiviral vectors are obtained by modifying the pol gene encoding the Integrase, resulting in a mutated pol gene encoding an integrative deficient integrase, said modified pol gene being contained in the encapsidation plasmid. Such integration-incompetent lentiviral vectors have been described in patent application WO 2006/010834. Accordingly the integrase capacity of the protein is altered whereas the correct expression from the encapsidation plasmid of the GAG, PRO and POL proteins and/or the formation of the capsid and hence of the vector particles, as well as other steps of the viral cycle, preceding or subsequent to the integration step, such as the reverse transcription, the nuclear import, stay intact. An integrase is said defective when the integration that it should enable is altered in a way that an integration step takes place less than 1 over 1000, preferably less than 1 over 10000, when compared to a lentiviral vector containing a corresponding wild-type integrase.

In a particular embodiment of the invention, the defective integrase results from a mutation of class 1, preferably amino acid substitutions (one-amino acid substitution) or short deletions fulfilling the requirements of the expression of a defective integrase.

The mutation is carried out within the pol gene. These vectors may carry a defective integrase with the mutation D64V in the catalytic domain of the enzyme, which specifically blocks the DNA cleaving and joining reactions of the integration step. The D64V mutation decreases integration of pseudotyped HIV-1 up to 1/10,000 of wild type, but keep their ability to transduce non dividing cells, allowing efficient transgene expression.

Other mutations in the pol gene which are suitable to affect the integrase capacity of the integrase of HIV-1 are the following: H12N, H12C, H16C, H16V, S81 R, D41A, K42A, H51A, Q53C, D55V, D64E, D64V, E69A, K71A, E85A, E87A, D116N, D116I, D116A, N120G, N120I, N120E, E152G, E152A, D-35-E, K156E, K156A, E157A, K159E, K159A, K160A, R166A, D167A, E170A, H171A, K173A, K186Q, K186T, K188T, E198A, R199C, R199T, R199A, D202A, K211A, Q214L, Q216L, Q221 L, W235F, W235E, K236S, K236A, K246A, G247W, D253A, R262A, R263A and K264H.

In a particular embodiment, mutation in the pol gene is performed at either of the following positions D64, D116 or E152, or at several of these positions which are in the catalytic site of the protein. Any substitution at these positions is suitable, including those described above.

Another proposed substitution is the replacement of the amino acids residues RRK (positions 262 to 264) by the amino acids residues AAH.

In a particular embodiment of the invention, when the lentiviral vector is integration-incompetent, the lentiviral genome further comprises an origin of replication (ori), whose sequence is dependent on the nature of cells where the lentiviral genome has to be expressed. Said origin of replication may be from eukaryotic origin, preferably of mammalian origin, most preferably of human origin. It may alternatively be of viral origin, especially coming from DNA circular episomic viruses, such as SV40 or RPS.

It is an advantageous embodiment of the invention to have an origin or replication inserted in the lentiviral genome of the lentiviral vector of the invention. Indeed, when the lentiviral genome does not integrate into the cell host genome (because of the defective integrase), the lentiviral genome is lost in cells that undergo frequent cell divisions; this is particularly the case in immune cells, such as B or T cells. The presence of an origin of replication ensures that at least one lentiviral genome is present in each cell, even after cell division, accordingly maximizing the efficiency of the immune response.

The lentiviral vector genome of said lentiviral vectors of the invention may especially be derived from HIV-1 plasmid pTRIPΔU3.CMV-GFP deposited at the CNCM (Paris, France) on Oct. 11, 1999 under number 1-2330 (also described in WO01/27300) or variants thereof. The sequence of such variants are provided as SEQ ID NO. 1 or 2.

When the vector genome is derived from these particular plasmids, a sequence of a recombinant polynucleotide encoding an antigenic polypeptide of a Plasmodium parasite as disclosed in the present application is inserted therein, in addition or in replacement of the GFP coding fragment. The GFP coding sequence may also be substituted by a different marker. The CMV promoter may also be substituted by another promoter, especially one of the promoters disclosed above, especially in relation to the expression of the transgene.

The WPRE sequence also contained in the particular deposited pTRIP vectors may optionally be deleted.

Vector particles may be produced after transfection of appropriate cells (such as mammalian cells or human cells, such as Human Embryonic Kidney cells illustrated by 293 T cells) by said plasmids, or by other processes. In the cells used for the expression of the lentiviral particles, all or some of the plasmids may be used to stably express their coding polynucleotides, or to transiently or semi-stably express their coding polynucleotides.

The concentration of particles produced can be determined by measuring the P24 (capsid protein for HIV-1) content of cell supematants.

The lentiviral vector of the invention, once administered into the host, infects cells of the host, possibly specific cells, depending on the envelope proteins it was pseudotyped with. The infection leads to the release of the lentiviral vector genome into the cytoplasm of the host cell where the retrotranscription takes place. Once under a triplex form (via the DNA flap), the lentiviral vector genome is imported into the nucleus, where the polynucleotide(s) encoding polypeptide(s) of antigen(s) of the malaria parasite is (are) expressed via the cellular machinery. When non-dividing cells are transduced (such as DC), the expression may be stable. When dividing cells are transduced, such as B cells, the expression is temporary in absence of origin of replication in the lentiviral genome, because of nucleic acid dilution and cell division.

The expression may be longer by providing an origin of replication ensuring a proper diffusion of the lentiviral vector genome into daughter cells after cell division. The stability and/or expression may also be increased by insertion of MAR (Matrix Associated Region) or SAR (Scaffold Associated Region) elements in the vector genome.

Indeed, these SAR or MAR regions are AT-rich sequences and enable to anchor the lentiviral genome to the matrix of the cell chromosome, thus regulating the transcription of the polynucleotide encoding at least one antigenic polypeptide, and particularly stimulating gene expression of the transgene and improving chromatin accessibility.

If the lentiviral genome is non integrative, it does not integrate into the host cell genome. Nevertheless, the at least one polypeptide encoded by the transgene is sufficiently expressed and longer enough to be processed, associated with MHC molecules and finally directed towards the cell surface. Depending on the nature of the polynucleotide(s) encoding antigenic polypeptide(s) of a malaria parasite, the at least one polypeptide epitope associated with the MHC molecule triggers a humoral or a cellular immune response.

Unless otherwise stated, or unless technically not relevant, the characteristics disclosed in the present application with respect to any of the various features, embodiments or examples of the structure or use of the lentiviral particles, especially regarding their envelope protein(s), or the recombinant polynucleotide, may be combined according to any possible combinations.

Further features and properties of the present invention, including features to be used in the embodiments described above will be described in the examples and figures which follow and may accordingly be used to characterise the invention.

### LEGENDS OF THE FIGURES

FIG. 1. Schema of plasmids used in the production of Lentiviral Particles.

FIG. 2. C57BL/6 mice (n=5) were immunized intramuscularly with 5×10⁷ TU of VSV^{IND} pseudotyped lentiviral particles coding for the antigens, CSP, Celtos SPECT, HSP20 and Ag13. As a positive control of protection, mice were immunized with 50 k irradiated sporozoites via intravenous injection. Thirty days after immunization, the animals were challenged with 10,000 bioluminescent sporozoites micro-injected subcutaneously in the mice footpad. The parasite load in the liver was quantified two days later by bioluminescence as shown in the picture for CSP, Celtos and Ag13. The graph shows the quantification of the liver infection represented as the log of average radiance (squares). Dotted line represents the average of background signal (Bk) of a non-infected region. *P<0.05 and ***P<0.001 (ANOVA).

FIG. 3. C57BL/6 mice (n=5 per group) were immunized or not (naïve) with 5×10⁷ TU of VSV^{IND} LPs carrying Ag13 (negative control) and CSP (positive control). The groups receiving concentrated LPs were inoculated intramuscularly in the thigh muscle with 50 uL of vector (Ag13 im c and CSP im c). The groups receiving non-concentrated LPs were inoculated intraperitoneally with 700 uL of vector (Ag13 ip nc and CSP ip nc). Thirty days after immunization, the animals were challenged with 5,000 luciferase-expressing sporozoites, micro-injected subcutaneously in the mice footpad. The parasite load in the liver was quantified two days later by bioluminescence as shown in the FIG. 2. The graph shows the average and sd of the log of average radiance in the liver two days after SPZ inoculation. Dotted line represents the average of background signal (Bk). ns, not significant (ANOVA).

FIG. 4. C57BL/6 mice (n=4-5 per group) were intraperitoneally immunized or not (naïve) with 1×10⁷ TU of non concentrated VSV^{IND} CSP LPs under the control of CMV or B2M promoters (CMV CSP and B2M CSP, respectively). Thirty days after immunization, the animals were challenged with 5,000 luciferase-expressing sporozoites micro-injected subcutaneously in the mice footpad. The parasite load in the liver was quantified two days later by bioluminescence as shown in the FIG. 2. The graph shows the average and sd of the log of average radiance in the liver two days after SPZ inoculation. Dotted line represents the average of background signal (Bk). *P<0.05; ns, not significant (ANOVA).

FIG. 5. 4 and 7 weeks-old C57BL/6 mice (n=4-per group) were acclimated for 3 weeks (old groups) and 3 days (new groups). These age-matched groups were then intraperitoneally immunized with 1×10⁷ TU of non concentrated VSV^{IND} B2M CSP or GFP LPs. Thirty days after immunization, the animals were challenged with 5,000 luciferase-expressing sporozoites micro-injected subcutaneously in the mice footpad. The parasite load in the liver was quantified two days later by bioluminescence as shown in the FIG. 2. The graph shows the average and sd of the log of average radiance in the liver two days after SPZ inoculation. Dotted line represents the average of background signal (Bk). *P<0.05; ns, not significant (ANOVA).

FIG. 6. 4 weeks-old C57BL/6 mice (n=4-per group) were acclimated for 3 weeks (old groups) and intraperitoneally immunized with different doses of non-concentrated VSV^{IND} B2M CSP (black) or GFP (white) LPs. Thirty days after immunization, the animals were challenged with 5,000 luciferase-expressing sporozoites micro-injected subcutaneously in the mice footpad. The parasite load in the liver was quantified two days later by bioluminescence as shown in the FIG. 2. The graph shows the average and sd of the log of average radiance in the liver two days after SPZ inoculation. Dotted line represents the average of background signal (Bk). *P<0.05; ***P<0.001; ns, not significant (ANOVA).

FIG. 7. Analysis of Sporozoite, Liver Stage and Blood Stage cDNA libraries of Plasmodium berghei (Pb) and falciparum (Pf) deposited in Plasmodb. The percentage of each expression sequence tag (EST) was normalized to the total number of ESTs and represented cumulatively. Each symbol represents one gene, ranked by EST abundance (higher to lower) and represented as % of total ESTs. Of note ˜10% of genes (most abundant) are responsible for ˜50% of total ESTs (dotted lines).

FIG. 8. Expression and surface localization of antigens. GFP-expressing Pb sporozoites were fixed with 2% of PFA and permeabilized with 0.1% of Triton X100 (perm) or not (live). Parasites were incubated with the indicated immune-sera (1/50) for one hour on ice, washed and revealed with goat anti-mouse secondary antibody labelled with AlexaFluor 647. Sporozoites were then analysed by cytometry as shown in the right histograms (surface, staining using live non-permeabilized SPZ; permeabilized, staining using fixed and permeabilized SPZ) or by fluorescence microscopy, as depicted in the pictures. Notice that CSP and antigen 9-6 present a surface pattern staining both by cytometry and microscopy.

FIG. 9. Targeted screening of protective antigens. 4 weeks-old C57BL/6 mice (n=5 per group) were acclimated for 3 weeks and intraperitoneally immunized with a single dose of 1×10⁷ TU of non-concentrated VSV^{IND} B2M LPs. Thirty days after immunization, the animals were challenged with 5,000 GFP-expressing sporozoites micro-injected subcutaneously in the mice footpad. The parasite infection was measured by flow cytometry. The graph shows the average of the log of parasitemia (trace, individual mice represented by circles) immunized with the indicated plasmodial antigens. Bold dotted lines represent the 95% tolerance interval of GFP log normal distribution. Mice with parasitemia below the lower limit of the tolerance interval are considered protected. Top dotted line is the average of control and bottom dotted line represents non-infected (NI) mice.

FIG. 10. Comparison of protection induced by one or two immunization doses. 4 weeks-old C57BL/6 mice (n=5 per group) were acclimated for 3 weeks and intraperitoneally immunized with a first dose of 5×10⁵ TU of non-concentrated VSV^{NJ}B2M LPs. Thirty days after the first immunization, the animals received a second dose of 1×10⁷ TU of non-concentrated VSV^{IND} B2M LPs. Thirty days later, mice were challenged with 5,000 GFP-expressing sporozoites micro-injected subcutaneously in the footpad. The parasite infection was measured by flow cytometry. The graph shows the log of parasitemia at day 5 post-inoculation of individual challenged mice that received two immunization doses (Squares, PB). Circles represent mice that received only one immunization dose of LPs (data from experiment shown in FIG. 9). Traces represents the average of the Log Parasitemia. Bold dotted lines represent the 95% tolerance interval of GFP log normal distribution. Mice below the lower limit of tolerance interval are considered protected. NI, non-infected mice.

FIG. 11. Targeted Screening of Protective Antigens. 4 weeks-old C57BL/6 mice (n=5-10 per group) were acclimated for 3 weeks and intraperitoneally immunized with a first dose of 5×10⁵ TU of non-concentrated VSV^{NJ} B2M LPs. Thirty days after the first immunization, the animals received a second dose of 1×10⁷ TU of non-concentrated VSV^{IND} B2M LPs. Third days later, mice were challenged with 5,000 GFP-expressing sporozoites micro-injected subcutaneously in the footpad. The parasite blood infection was measured by flow cytometry. (a.) The upper graph shows the log of parasitemia of individual mouse at day 5 post-infection. Traces represent the mean of the log parasitemia. The average of the GFP group (control of protection) is represented by the dotted middle line. The superior and inferior dotted lines delineate the 95% tolerance interval (grey box) of the GFP control group. The CSP group is the positive control of protection. NI (not infected=no parasitemia at day 10 post-infection, located at the limit of detection of our method of parasitemia quantification). Black circles represent antigens where there was a significant decrease in the averaged log parasitemia and therefore are considered protective (ANOVA). (b) The bottom graph represents the percentage of protected mice (% of animals below the 95% tolerance interval). Black bars represent protective antigens (Fisher's Exact test). *P<0.05, **P<0.01, ****P<0.0001.

FIG. 12. Structure of P. berghei protective antigens. Conserved structural and functional domains are represented by boxes according to the code on the right. GPI (glycosylphosphatidylinositol), TSR (thrombospondin type I repeat), MACPF (membrane attack complex/perforin).

FIG. 13. Protective antigens are conserved among plasmodial species. Amino acid sequence of protective orthologous antigens from rodent-infecting P. berghei, macaque-infecting P. cynomolgi, and human-infecting P. falciparum and P. vivax parasites were aligned by MUltiple Sequence Comparison by Log-Expectation (MUSCLE). Vertical black bars represent identical amino acids conserved in the four plasmodial species, short dark gray bars represent repetitive regions and short light gray bars, insertional gaps used for the alignment.

FIG. 14. Protection induced by combination of down-selected protective antigens with a sub-optimal dose of CSP. Mice were immunized twice, four weeks apart, with a sub-optimal dose of CSP (5×10 TU of non-concentrated VSV^{NJ} B2M LP in the first immunization and 5×10⁶ TU of non-concentrated VSV^{IND} B2M LP in the second immunization, white triangle, CSP) and the usual dose of protective plasmodial antigens (CSP+11-03, +11-05, +11-06, +11-07, +11-09 and +11-10; triangles). As negative control mice were immunized with the usual, two doses of GFP. 4 weeks after the second immunization dose, animals were challenged with 5,000 sporozoites.

FIG. 15. Sterile protection induced by a multigenic combination. Mice were immunized twice, four weeks apart, with 7× the individual dose (1 dose=5×10⁵ TU of non-concentrated VSV^{NJ} B2M LPs in the first immunization/1×10⁷ TU of non-concentrated VSV^{IND} B2M LPs in the second immunization) of the control antigen AL11-luciferase (Luc, white triangles), with the individual dose of CSP plus 6×Luc (gray triangles), or with the individual doses of CSP and of 6 conserved PE antigens (11-05, 11-06, 11-07, 11-09, 11-10 and 18-10; black triangles, 7cPEAg). 4 weeks after the second immunization dose, mice were challenged with 5,000 GFP SPZs. Both graphs show the individual log of parasitemia at day 5 post-challenge. (a) The graph shows the pooled results of three independent experiments. Number of sterile protected/challenged mice: 7×Luc (0/21), 1×CSP 6×Luc (1/20) and 1×7cPEAg (18/21). (b) Three and one day before sporozoite challenge, 1×7cPEAg immunized mice were injected with 400 μg of control (Ctr), CD4-depleting (a-CD4+, clone GK1.5) and CD8-depleting (a-CD8+, clone 2.43) monoclonal antibodies. GFP data comes from experiment showed in FIG. 11 (gray circles). Number of sterile protected/challenged mice: 7×Luc (0/7), 1×CSP 6×Luc (1/7) and 1×7cPEAg (ctr,7/7; a-CD8, 0/7 and a-CD4, 7/7). Notice that depletion of CD8+ cells abolished sterile protection. *P<0.05, **P<0.01, ****P<0.0001 (ANOVA).

FIG. 16. Sterile protection induced by a multigenic combination in a single immunization dose. (a) Mice were immunized twice, four weeks apart, with 7× the individual dose (1 dose=5×10⁵ TU of non-concentrated VSV^{NJ} B2M LPs in the first immunization/1×10⁷ TU of non-concentrated VSV^{IND} B2M LPs in the second immunization) of the control antigen AL11-luciferase (Luc, black triangles), with the individual dose of CSP plus 6×Luc (CSP, black triangles), or with the individual dose of CSP and of 6 conserved PE antigens (11-05, 11-06, 11-07, 11-09, 11-10 and 18-10; black triangles; 2 im 7cPEAg). Alternatively, mice were administered only with the second individual immunization dose (1×10⁷ TU) of CSP and of 6 conserved PE antigens (11-05, 11-06, 11-07, 11-09, 11-10 and 18-10; grey diamonds; 1 im 7cPEAg). 4 weeks after the second immunization dose, mice were challenged with 5,000 GFP SPZs. The graph shows the individual log of parasitemia at day 5 post-challenge. Black bars are the average of log of parasitemia. Number of sterile protected/challenged mice: Luc (0/7), CSP (0/7), 2im 7cPEAg (6/7) and 1m 7cPEAg (6/7). (b) Mice were immunized once with 9× the individual dose (1 dose=1×10⁷ TU of non-concentrated VSV^{ND} B2M LPs) of the control antigen AL11-luciferase (Luc, black diamonds), or with the individual doses of CSP+ of 7 conserved PE antigens (11-05, 11-06, 11-07, 11-09, 11-10, 18-10, 30-03A and 30-03B; grey diamonds; 8cPEAg). Three and one day before sporozoite challenge, 8cPEAg immunized mice were injected with 400 μg of control (Ctr), CD4-depleting (a-CD4+, clone GK1.5) and CD8-depleting (a-CD8+, clone 2.43) monoclonal antibodies. 4 weeks after the single immunization dose, mice were challenged with 5,000 GFP SPZs. The graph shows the individual log of parasitemia at day 5 post-challenge. Black bars are the average of log of parasitemia. Number of sterile protected/challenged mice: 7×Luc (0/7) and 8cPEAg (ctr,6/7; a-CD8, 0/7 and a-CD4, 4/7). Notice that depletion of CD8+ cells abolished protection. *P<0.05; **P<0.01; ****P<0.0001; ns, P>0.05 (ANOVA).

FIG. 17. Sterile protection induced by a minimal combination of 5 PE antigens. (a) Mice were immunized twice, four weeks apart, with the individual dose multiplied by the number indicated in the circles (1 dose=5×10⁵ TU of non-concentrated VSV^{NJ} B2M LPs in the first immunization/1×10⁷ TU of non-concentrated VSV^{IND} B2M LPs in the second immunization). For example, for the control antigen AL11-luciferase (LUC), animals were immunized with 5× the individual dose. All groups received 5 doses, with exception of the positive control of protection that received 7 doses of LPs (7PEAg). 4 weeks after the second immunization dose, mice were challenged with 5,000 GFP SPZs. Bars represents the percentage of sterile protected mice. The numbers of sterile protected/challenged mice are shown at the right of bars. **P<0.01 (Fisher's Exact test). (b) Mice were immunized twice, four weeks apart, with the individual dose (1 dose=5×10⁵ TU of non-concentrated VSV^{NJ} B2M LPs in the first immunization/1×10⁷ TU of non-concentrated VSV^{IND} B2M LPs in the second immunization) of the control antigen GFP (GFP, black circles) or with the individual dose of CSP, TRAP, 18-10, 11-09 or 11-10 (grey triangles). Three and one day before sporozoite challenge, immunized mice were injected with 400 μg of control (Ctr), CD4-depleting (a-CD4+, clone GK1.5) and CD8-depleting (a-CD8+, clone 2.43) monoclonal antibodies. 4 weeks after the second immunization dose, mice were challenged with 5,000 GFP SPZs. Graphs show the average ±sd of log of parasitemia at day 5 post-challenge. *P<0.05; ns, P>0.05 (ANOVA). (c) The 5 down-selected protective antigens were split according the presence of predicted CD8 T cell epitopes and respecting conserved structural domains as depicted by the schematic representation of the antigens. The graphs above the schematic proteins represent the distribution of epitopes predicted to bind to H2Kb (8 aa) and H2Kd (9 aa) MHC class I molecules using SYFPEITHI (score) and IEDB ANN IC 50 (nM). The graphs on the left of schematic proteins represent the protection induced by these constructs, where bars are the average ±sd of log of parasitemia at day 5 post-challenge. Data shown for antigen 11-09 come from FIG. 11. Dotted line represents the inferior limit of the tolerance interval of the control calculated in the FIG. 11. *P<0.05; ns, P>0.05 (ANOVA). (d) Correlation of the best epitope predicted to bind to MHC class I molecules in the segments of CD8+ T cell dependent PE antigens and mean protective activity obtained from 17c. Circles show the IC50 using IEDB ANN software and squares the score values using SYFPEITHI. Dotted line shows the average of Luc control.

FIG. 18. Clustering of CD8 T cell epitopes in conserved amino acid regions and binding of predicted Pf epitopes to HLA A02:01. Amino acid sequences of protective orthologous antigens from rodent-infecting P. berghei, macaque-infecting P. cynomolgi, and human-infecting P. falciparum and P. vivax parasites were aligned by MUltiple Sequence Comparison by Log-Expectation (MUSCLE). Vertical black bars represent identical amino acids conserved in the four plasmodial species. The graph shows the distribution of Pb epitopes predicted to bind to H2Kb (8 aa) and H2Kd (9 aa) MHC class I molecules or of Pf epitopes predicted to bind to the HLA A02:01 (9 mers) using SYFPEITHI (score) and IEDB ANN IC50 (nM). The best predicted HLA binders were tested in the assay of stabilization of MHC class I molecule in the presence of peptide and β2-microglobulin (REVEAL® Score). The score of 100 corresponds to the binding of a positive control peptide. Notice the clustering of epitopes in regions of conserved amino acids.

FIG. 19. plasmid used to produce VSV-pseutdotyped lentiviral particles: pTRIP CMV GFP

The sequence of the plasmid is constituted by the following functional regions wherein the cis-active lentiviral regions are derived from the HIV genome, and the promoter driving the expression of the protein (GFP) is CMV:

The insert in the plasmid that provides the vector genome is composed as follows: LTR-Ψ-RRE-cPPT/CTS-CMV-GFP-WPRE-ΔU3LTR, wherein

| | |
|---|---|
| LTR | is Long Terminal Repeat |
| Psi (Ψ) | is Packaging signal |
| RRE | is Rev Responsive Element |
| CMV | is Immediate early CytoMegaloVirus promoter |
| cPPT | is central PolyPurine Tract, and wherein the nucleotide segment |
| | from cPPT to CTS forms the flap sequence |
| CTS | is Central Termination Sequence |
| WPRE | is Woodchuck hepatitis virus Post Regulatory Element |

The nucleotide sequence is provided as SEQ ID No. 1

FIG. 20: alternative plasmid (to the plasmid of FIG. 16) used to produce VSV-pseutdotyped lentiviral particles: pTRIP B2M GFP

The insert in the plasmid that provides the vector genome is composed as follows: LTR-Ψ-RRE-cPPT/CTS-B2M-GFP-WPRE-ΔU3LTR.

The nucleotide sequence is provided as SEQ ID No. 2

FIG. 21. plasmid used to produce VSV-pseutdotyped lentiviral particles: packaging 8.74 plasmid

The plasmid provides the required GAG and POL coding sequences of the HIV-1 lentivirus under the control of the CMV promoter.

The nucleotide sequence is provided as SEQ ID No. 3.

FIG. 22. plasmid used to produce VSV-pseutdotyped lentiviral particles: encapsidation plasmid pCMV-VSV-INDco

The envelope protein is the VSV-G of the Indiana strain and the coding sequence has been mouse-codon optimized.

The nucleotide sequence is provided as SEQ ID No. 4.

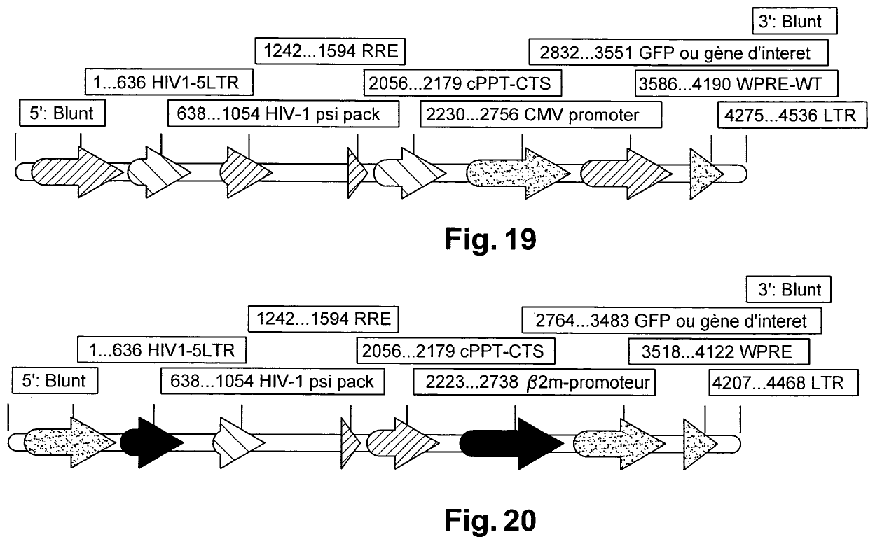

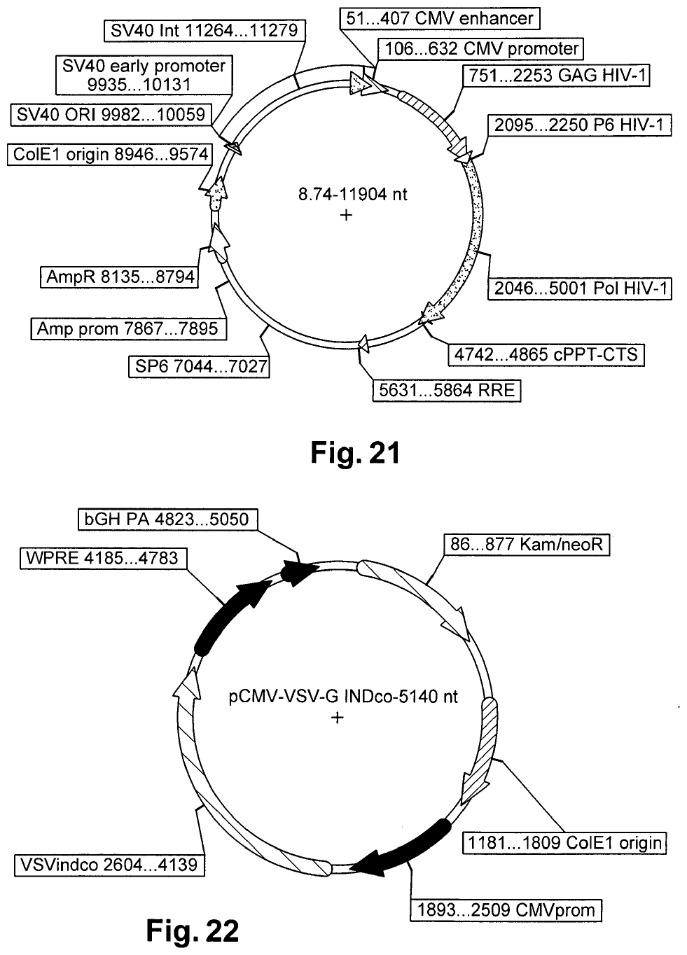

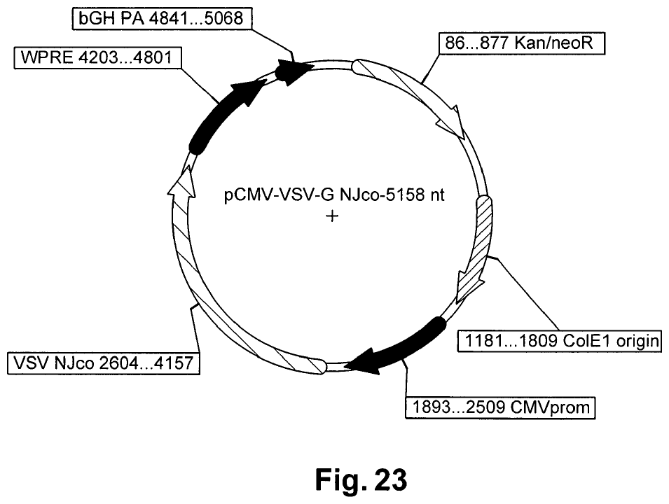

FIG. 23. alternative plasmid (to plasmid of FIG. 19) used to produce VSV-pseutdotyped lentiviral particles: encapsidation plasmid pCMV-VSV-NJco

The envelope protein is the VSV-G of the New-Jersey strain and the coding sequence has been mouse-codon optimized.

The nucleotide sequence is provided as SEQ ID No. 5.

The following table provides the list and identification of the sequences contained in the sequence listing.

| SEQ ID | | | |
|---|---|---|---|
| No. | Sequence designation | Origin | Type |
| 1 | pTRIP CMV GFP | | DNA |
| 2 | pTRIP B2M GFP | | DNA |
| 3 | PACKAGING 8.74 PLASMID | | DNA |
| 4 | pCMV-VSV-INDco | | DNA |
| 5 | pCMV-VSV-Njco | | DNA |
| 6 | eGFP | | DNA |
| 7 | eGFP protein | | protein |
| 8 | AL11-Luciferase | | protein |
| 9 | AL11-Luciferase | | protein |
| 10 | circumsporozoite (CS) protein (CSP) mouseCO + | P. berghei | DNA |
| | Kozak | ANKA strain | |
| 11 | PbCSP (mouseCO + Kozak) | P. berghei | protein |
| | | ANKA strain | |
| 12 | PbCSP | P. berghei | protein |
| | | ANKA strain | |
| 13 | PfCSP humanCO + Kozak | P falciparum | DNA |
| | | 3D7 strain | |
| 14 | PfCSP (humanC0 + Kozak) | P falciparum | protein |
| | | 3D7 strain | |
| 15 | PfCSP | P falciparum | protein |
| 16 | PvCSP humanCO + Kozak | P vivax Sal-1 | DNA |
| | | strain | |
| 17 | PvCSP (humanCO + Kozak) | P vivax Sal-1 | protein |
| | | strain | |
| 18 | PvCSP | P vivax Sal-1 | protein |
| | | strain | |
| 19 | thrombospondin-related anonymous protein | P. berghei | DNA |
| | (PbTRAP) mouseCO + Kozak | ANKA strain | |
| 20 | PbTRAP (mouseCO + Kozak) | P. berghei | protein |
| | | ANKA strain | |
| 21 | PbTRAP | P. berghei | protein |
| | | ANKA strain | |
| 22 | PfTRAP humanCO + Kozak | P falciparum | DNA |
| | | 3D7 strain | |
| 23 | PfTRAP (humanCO + Kozak) | P falciparum | protein |
| | | 3D7 strain | |
| 24 | PfTRAP | P falciparum | protein |
| 25 | PvTRAPhumanCO | P vivax Sal-1 | DNA |
| | | strain | |
| 26 | PvTRAP | P vivax Sal-1 | protein |
| | | strain | |
| 27 | PvTRAP | P vivax | protein |
| 28 | inhibitor of cysteine proteases (ICP) mouseCO + | P. berghei | DNA |
| | Kozak | ANKA strain | |
| 29 | PbICP (mouseCO + Kozak) | P. berghei | protein |
| | | ANKA strain | |
| 30 | PbICP | P. berghei | protein |
| | | ANKA strain | |
| 31 | PfICP humanCO | P falciparum | DNA |
| | | 3D7 strain | |
| 32 | PfICP | P falciparum | protein |
| | | 3D7 strain | |
| 33 | PfICP | P falciparum | protein |
| 34 | PvICP humanCO + Kozac | P vivax Sal-1 | DNA |
| | | strain | |
| 35 | PvICP (humanCO + Kozac) | P vivax Sal-1 | protein |
| | | strain | |
| 36 | PvICP | P vivax | protein |
| 37 | Bergheilysin-A-mouseCO + Kozak | P. berghei | DNA |
| | | ANKA strain | |
| 38 | Bergheilysin-A (1-777, mouse CO + Kozak) | P. berghei | protein |
| | | ANKA strain | |
| 39 | Bergheilysin entire ORF (1-1149) | P. berghei | protein |
| | | ANKA strain | |
| 40 | Falcilysin human CO + Kozak | P falciparum | DNA |
| | | 3D7 strain | |
| 41 | Falcilysin (human CO + Kozak) | P falciparum | protein |
| | | 3D7 strain | |
| 42 | Falcilysin | P falciparum | protein |
| | | 3D7 strain | |
| 43 | PvFalcilysin human CO + Kozak | P vivax Sal-1 | DNA |
| | | strain | |
| 44 | PvFalcilysin (humanCO + Kozak) | P vivax Sal-1 | protein |
| | | strain | |
| 45 | PvFalcilysin | P vivax Sal-1 | protein |
| | | strain | |
| 46 | Bergheilysin-B-mouseCO + Kozak + signal | P. berghei | DNA |
| | peptide (SP) | ANKA strain | |
| 47 | Bergheilysin-B (SP + 778-1149, mouse CO + | P. berghei | protein |
| | Kozak) | ANKA strain | |
| 48 | perforin like protein 1 (SPECT2) mouseCO + | P. berghei | DNA |
| | Kozak | ANKA strain | |
| 49 | PbSPECT2 (mouseCO + Kozak) | P. berghei | protein |
| | | ANKA strain | |
| 50 | PbSPECT2 | P. berghei | protein |
| | | ANKA strain | |
| 51 | PfSPECT2 human CO + Kozak | P falciparum | DNA |
| | | 3D7 strain | |
| 52 | PfSPECT2 (humanCO + Kozak) | P falciparum | protein |
| | | 3D7 strain | |
| 53 | PfSPECT2 | P falciparum | protein |
| 54 | PvSPECT2 human CO + Kozak | P vivax Sal-1 | DNA |
| | | strain | |
| 55 | PvSPECT2 (human CO + Kozak) | P vivax Sal-1 | protein |
| | | strain | |
| 56 | PvSPECT2 | P vivax | protein |
| 57 | GPI_P113 mouseCO + Kozak | P. berghei | DNA |
| | | ANKA strain | |
| 58 | Pb GPI_P113 (mouseCO + Kozak) | P. berghei | protein |
| | | ANKA strain | |
| 59 | Pb GPI_P113 | P. berghei | protein |
| | | ANKA strain | |
| 60 | PfP113 human CO + Kozak | P falciparum | DNA |
| | | 3D7 strain | |
| 61 | PfP113 (human CO + Kozak) | P falciparum | protein |
| | | 3D7 strain | |
| 62 | P113 | P falciparum | protein |
| 63 | PvP113 human CO + Kozak | P vivax Sal-1 | DNA |
| | | strain | |
| 64 | PvP113 (human CO + Kozak) | P vivax Sal-1 | protein |
| | | strain | |
| 65 | P113 | P vivax | protein |
| 66 | PbAg40 mouse CO + Kozak | P. berghei | DNA |
| | | ANKA strain | |
| 67 | PbAg40 (mouse CO + Kozak) | P. berghei | protein |
| | | ANKA strain | |
| 68 | PbAg40 | P. berghei | protein |
| | | ANKA strain | |
| 69 | PfAg40 human CO + Kozak | P falciparum | DNA |
| | | 3D7 strain | |
| 70 | PfAg40 (human CO + Kozak) | P falciparum | protein |
| | | 3D7 strain | |
| 71 | Ag40 | P falciparum | protein |
| 72 | PvAg40 human CO + Kozak | P vivax Sal-1 | DNA |
| | | strain | |
| 73 | PvAg40 (human CO + Kozak) | P vivax Sal-1 | protein |
| | | strain | |
| 74 | PvAg40 | P vivax Sal-1 | protein |
| | | strain | |
| 75 | PbAg45 mouse CO + Kozak | P. berghei | DNA |
| | | ANKA strain | |
| 76 | PbAg45 (mouse CO + Kozak) | P. berghei | protein |
| | | ANKA strain | |
| 77 | PbAg45 | P. berghei | protein |
| | | ANKA strain | |
| 78 | PfAg45 human CO + Kozak | P falciparum | DNA |
| | | 3D7 strain | |
| 79 | PfAg45 (human CO + Kozak) | P falciparum | protein |
| | | 3D7 strain | |
| 80 | PfAg45 | P falciparum | protein |
| 81 | PvAg45 human CO + Kozak | P vivax Sal-1 | DNA |
| | | strain | |
| 82 | PvAg45 (human CO + Kozak) | P vivax Sal-1 | protein |
| | | strain | |
| 83 | PvAg45 | P vivax | protein |
| 84 | Kozak consensus sequence | | DNA |
| 85 | Kozak consensus sequence | | DNA |
| 86 | BamHI site | | DNA |
| 87 | Xhol site | | DNA |
| 88-94 | CD8 T cell epitopes | | protein |

Additional information relating to some of the sequences disclosed in the above table are provided in the table below.

| SEQ ID | GenBank | strain | pubmed |
|---|---|---|---|
| 15 | BAM84930.1 | Plasmodium falciparum isolate PaI97-042 | 23295064 |
| | | origin: Philippines | |
| | ACO49323 | Plasmodium falciparum” isolate A5 | 19460323 |
| | | origin: Thailand | |
| 18 | AAA29535.1 | P. vivax (strain Thai; isolate NYU Thai) | 2290443 |
| | | origin: Thailand | |
| 24⁽¹⁾ | EWC74605.1 | Plasmodium falciparum UGT5.1 strain | |
| | | origin: Uganda | |
| 27 | AIU97014.1 | Plasmodium vivax isolate = “TMS38” | |
| | | origin: Thailand | |
| 36⁽³⁾ | KMZ87332.1 | Plasmodium vivax Brazil I strain | |
| 56 | KMZ82648.1 | Plasmodium vivax India VII | |
| 65 | KMZ78214.1 | Plasmodium vivax India VII | |
| 83 | KMZ90984.1 | Plasmodium vivax Mauritania I | |

⁽¹⁾https://www.ncbi.nlm.nih.gov/biosample/SAMN01737342
(2): https://www.ncbi.nlm.nih.gov/biosample/SAMEA2394724
⁽³⁾https://www.ncbi.nlm.nih.gov/biosample/SAMN00710434

### Examples

To approach the complex problem of identifying protective antigens, the inventors devised a functional screening to identify and combine novel PE protective antigens using a rodent malaria model where mice (C57BL/6) are extremely susceptible to Plasmodium berghei (Pb) sporozoite infection. In this model, sterilizing protection induced by live irradiated sporozoites is mediated by antibodies and mainly by CD8 T cell responses against sporozoites and liver stages, respectively. The inventors' screening strategy was designed based on four main features: i) parameterized selection of 55 PE antigens based on abundance, orthology, predicted topology and function, ii) synthesis of codon-optimized antigens to avoid AT-rich plasmodial sequences and maximize the expression in mammalian cells, iii) immunization using HIV-based lentiviral vector that elicits strong humoral and cellular responses^{11,12}, and iv) measurement of protection after a stringent challenge of sporozoites inoculated sub-cutaneously in the immunized mice.

### 1. Setting Up the Parameters of the Screening.

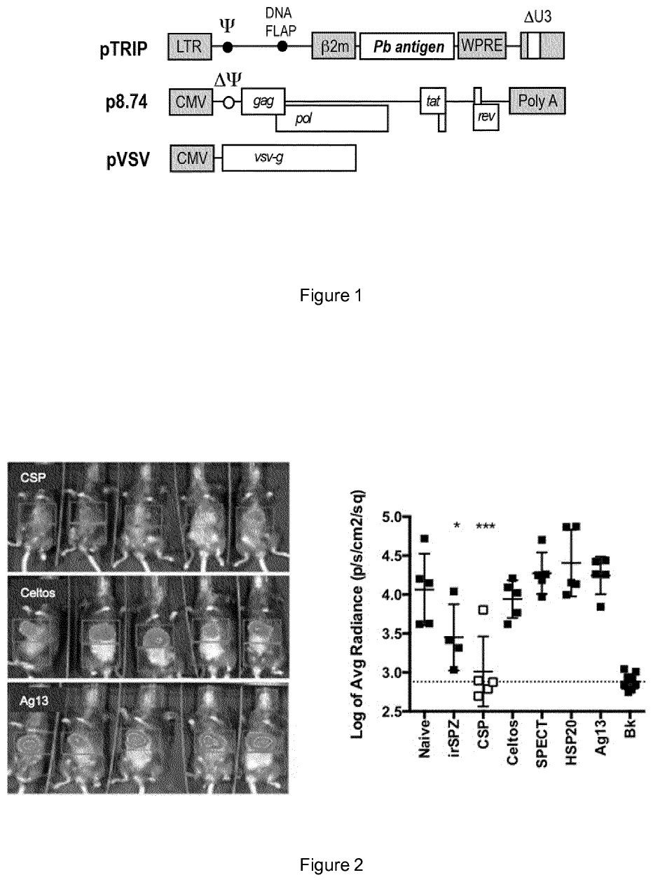

In a proof-of-concept experiment aimed at validating the viability of our strategy to screen antigens at a medium-throughput, the inventors ordered mouse-codon optimized synthetic genes of Pb CSP (SEQ ID No. 11 and 12), a known protective antigen, and of more 4 other sporozoite antigens (Celtos, SPECT, HSP20 and Ag13), which were previously correlated with protection¹³. The synthetic plasmodial genes were cloned in to the pTRIP vector plasmid, which drives their expression in mammalian cells via the immediate-early cytomegalovirus promoter (CMV) and the post-transcriptional regulatory element of woodchuck hepatitis virus (WPRE) (FIG. 1). These two elements assure a strong expression of the antigen in a wide variety of mouse cells in vivo. HIV-1 derived lentiviral particles were produced by transient co-transfection of HEK 293T cells with three helper plasmids encoding separate packaging functions, the pTRIP vector plasmid containing the synthetic plasmodial gene, the envelope expression plasmid encoding the glycoprotein G from the Vesicular Stomatitis Virus, Indiana (VSV^{IND}) or New Jersey (VSV^{NJ}) serotypes, and the p8.74 encapsidation plasmid (FIG. 1). This co-transfection generates integrative but replication-incompetent pseudotyped lentiviral particles capable of transducing dividing and non-dividing cells—including dendritic cells—and inducing potent cellular⁶ and humoral⁷ memory responses. The particles were collected 48 hours after co-transfection and each batch of vector were titrated in HeLa cells by quantitative PCR. This functional titration assay gives the concentration of particles capable to transfer one copy of the gene per cell and will be expressed in Transducing Units (TU)/mL. Plasmid sequences are shown in the figures and their sequences are provided in the sequence listing.

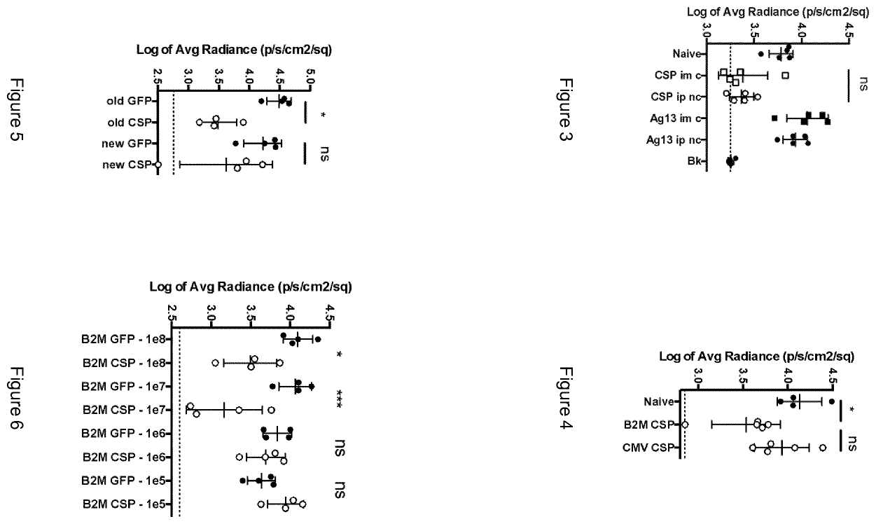

Groups of five mice were immunized with a single intra-muscular dose of 5e7 TU of ultracentrifugation-concentrated vsV^{1N} pseudotyped lentiviral particles (LPs). Thirty days after immunization, mice were challenged with 10,000 bioluminescent sporozoites inoculated sub-cutaneously in the footpad. Two days later, the parasite load in the liver was measured by bioluminescence. Surprisingly, CSP-immunization decreased 15×-fold the parasite load in the liver after a challenge using 10,000 bioluminescent sporozoites, versus a 5×-fold decrease in animals immunized intravenously with 50,000 irradiated sporozoites, our golden standard of protection (FIG. 2). This preliminary and promising result validated the high performance of the present method to functionally identify new protective antigens and showed the feasibility to scale-up our test samples.

The inventors next aimed at the transposition of these optimal experimental conditions to those of a larger screening. This transposition included the validation of the use of non-concentrated LPs, the choice of the best promoter driving the expression of the plasmodial antigens, and the dose of immunization.

The first parameter tested was the use of non-concentrated, instead of concentrated LPs, to avoid a costly and time consuming ultracentrifugation concentration step in the protocol of LP production, which requires large volumes of non-concentrated LP suspensions. FIG. 3 shows that there is no significant difference between protection induced by the same dose (5×10⁷ TU) of concentrated LPs injected intramuscularly (CS im c, 50 μL) and non-concentrated LPs injected intraperitoneally (CS ip nc, 700 μL). Protection was measured by reduction in the liver infection, as assessed by bioluminescence after a challenge of 5,000 sporozoites injected subcutaneously 30 days following immunization. As negative control of protection the inventors used mice immunized with Pb Ag13, determined previously as a non-protective antigen (FIG. 1).

Next two promoters were tested to identify which one induced the best protection using the codon optimized Pb CSP. The inventors compared the use of the strong and constitutive cytomegalovirus (CMV) promoter versus a human beta-2 microglobulin (B2M) promoter, which direct gene expression in many cell types, particularly in dendritic cells. FIG. 4 shows that CSP-induced protection was slightly better, although not statistically significant, using the B2M promoter at an immunization dose of 1×10⁷ TU of non-concentrated LP. Therefore the inventors further adopted this promoter in our constructs.

During this period of optimization the inventors observed some variations in the CSP-induced protection using the same stock of LPs, as can be seen in the FIG. 4. The inventors asked if this variability could be due to the process of mouse acclimation, including the modification of mouse microbiota. To test this hypothesis a group of mice purchased from Elevage Janvier (4 weeks-old) was reared in the animal facility for 3 weeks before immunization (group old). A second group of mice (7 weeks-old) was purchased and put in cages 3 days before the immunization (group new). Both groups were intraperitoneally immunized with 1×10⁷ TU of non-concentrated LPs. As shown in the FIG. 5, mouse acclimation of 3 weeks resulted in a significant and more homogeneous protection when compared to 3 days of acclimation. Consequently, the inventors adopted this period of acclimation in all our subsequent experiments.

Next, the best protective immunization dose was tested, ranging from 1×10⁸ to 1×10⁵ TU of B2M CSP non-concentrated LPs. As shown in FIG. 6, significant protection was observed using 10⁷ and 10⁸ TU, and the best protective activity was observed using an immunization dose of 1×10⁷ TU. In this experiment the inventors also observed a gradual loss of SPZ infectivity over time, as evidenced in the GFP groups, due to the use of a single SPZ stock to challenge all animals. To reduce the multiple shocks of temperature due to the manipulation of the stock tube, kept on ice between injections, the inventors prepared a SPZ stock for each group in the subsequent challenges and this variation disappeared.

In summary, an immunization protocol was set up based on CSP that relied on a single intraperitoneal injection of 10⁷ TU of non-concentrated VSV^{IND} B2M LP in C57BL/6 mice of 7 weeks-old, acclimated for 3 weeks in the animal facility. In the pooled data, this protocol leaded in average to a ˜5-fold decrease in the parasite liver load, as assessed by bioluminescence imaging, using a subcutaneous challenge of 5,000 luciferase-expressing SPZ.

However, this bioluminescent method of detection of parasites presents some disadvantages such as the use and associated costs of anesthesia and luminescent substrate, limited capacity of analysis of a few animals per acquisition, being time-consuming and not sensible enough to predict sterile protection. Therefore, the inventors decided to use fluorescent parasites to check protection by measuring parasitemia at day 4, 5, 6 and 10 post-inoculation by flow cytometry. The inventors analyze at least 100,000 red blood cells, which gives the sensibility to detect a parasitemia of 0.001%. At day 4 to 6, parasites grow exponentially in the blood therefore the log transform of parasitemia can be fitted using a linear regression where the slope represents the time of parasite replication per day. Consequently, the inventors use this parameter to determine if the immunization impacts the parasite growth in the blood. For quantifying protection the inventors use the log of parasitemia at day 5 post inoculation. This represents an indirect measure of liver infection and it is more robust than the measure at day 4 because more events of infected blood cells are registered. Finally the inventors defined that immunized mice are sterile protected if infected red blood cells are not detected after 10 days post inoculation. After defining the protocol of immunization and the method for the quantification of parasite infection the inventors started to screen the protective activity of down-selected antigens.

### 2. Parameterized Selection of Antigens

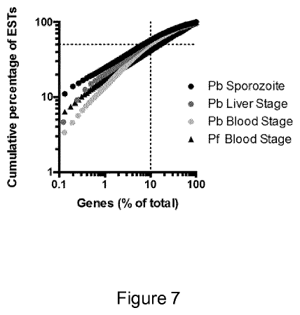

By merging proteomic and transcriptomic data using PlasmoDB (www.plasmodb.org), the inventors identified ˜9000 genes expressed in plasmodial pre-erythrocytic stages—salivary gland sporozoites and liver-stages—of three different plasmodial species, with 3654 syntenic orthologs in Plasmodium falciparum (Pf), the most lethal human-infecting plasmodial species. By analyzing the repertoire of pathogen transcripts, as inferred by the amount of expressed sequence tags (ESTs) obtained in cDNA libraries of different stages and species of malaria parasites, the inventors have observed that ˜50% of the total amount of ESTs are coming from only ˜10% of genes represented in these libraries, corresponding to approximately 100 genes in these libraries (FIG. 7). Therefore, by focusing on the ˜100 most abundant transcribed genes the inventors could target about 50% of the putative (to be translated) antigenic mass of a given parasite stage. Accordingly, the inventors selected ˜50 abundantly transcribed genes coding for conserved proteins with high probability of being expressed/presented on the surface of the parasite/infected cell, giving priority to candidates containing T cell epitopes predicted by IEDB MHC binding algorithm (http://tools.iedb.org/mhcil). A Kozak consensus sequence, a translational start site, was added to these down-selected genes, which were then mammalian codon-optimized and synthesized by MWG Eurofins (listed in the figures). These synthetic codon-optimized down-selected plasmodial genes were then cloned into the B2M pTRIP plasmid and produced as non-concentrated VSV^{IND} LPs.

### 3. First Screening of Protective Antigens Using a Single Dose of LPs

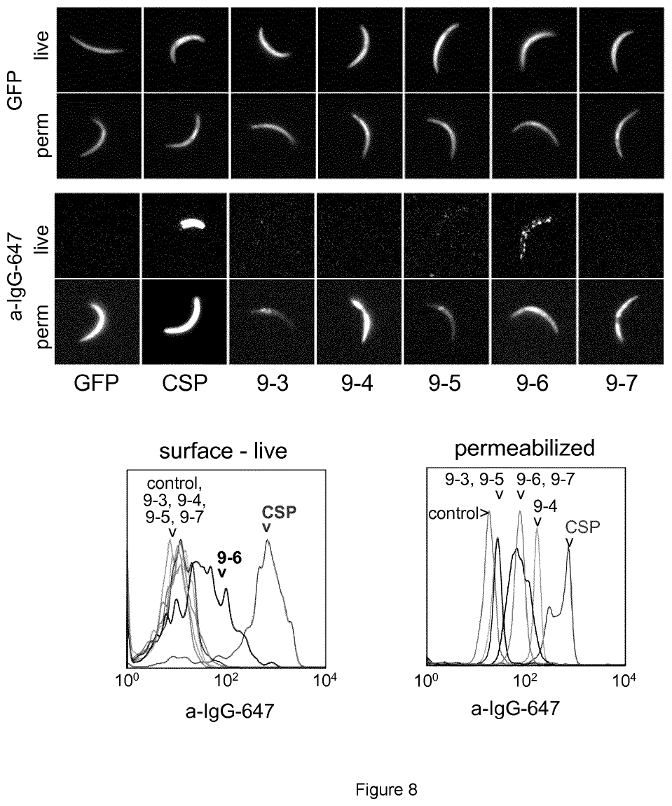

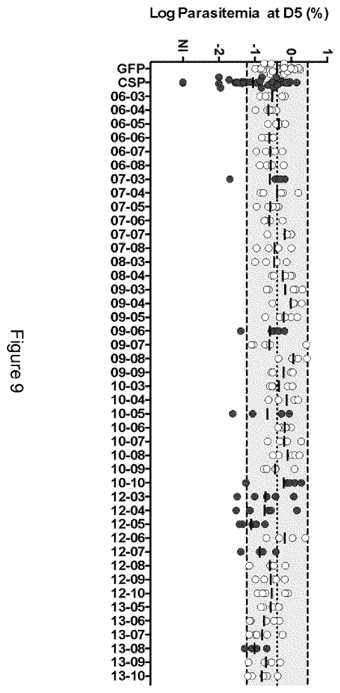

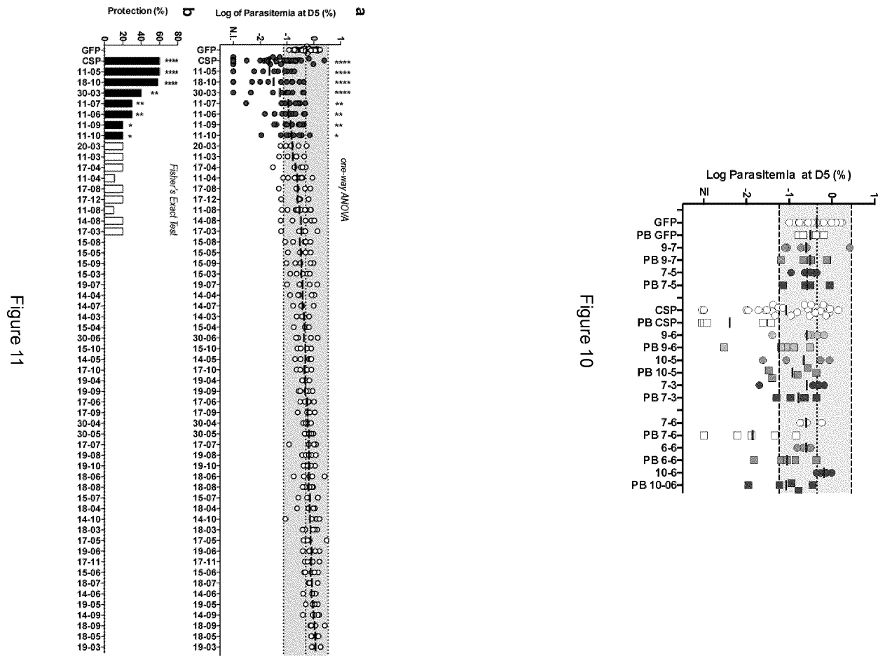

Usually, 6-10 plasmodial antigens were tested by experiment, with a negative (GFP) and positive (CSP) control of protection. After three weeks post-immunization, the immune-sera were tested on permeabilized and non-permeabilized sporozoites, allowing the determination of (i) the efficiency of the host humoral response and therefore the immunogenicity of the lentivirus-delivered antigen, and (ii) the localization of the parasite antigen (surface vs intracellular). As shown in the FIG. 8, where the inventors immunized mice with putative GPI-anchored antigens, surface antigens were identified by flow cytometry and immunofluorescence (CSP and 9-6). The sera of GFP and CSP group served, respectively, as positive control for intracellular and surface antigen localization.

Four weeks post-immunization the animals were challenged with 5,000 GFP-expressing sporozoites, microinjected in the footpad of immunized mice. Parasitemia was determined by flow cytometry. To define protection, parasitemia of all GFP groups (day 5 post-infection, n=35) was log transformed, pooled and the 95% tolerance interval was calculated (FIG. 9). All animals below the inferior limit of the tolerance interval, which represents a ˜8-fold decrease in parasitemia compared to the mean log of parasitemia of the GFP group, were considered protected. As positive control, 43% of animals (15/35) were protected by CSP immunization with a mean fold decrease of ˜5 fold in comparison to the GFP group. 9% of them (3/35) became sterile protected after sporozoite challenge.

In the first set of 43 antigens tested (FIG. 9), the inventors identified 9 PE antigens that protected at least one out of five immunized mice (black circles; 07-03, 09-06, 10-05, 10-10, 12-03, 12-04, 12-05, 12-07 and 13-08). Three of them were also identified as sporozoite surface antigens (09-06, 10-05 and 10-10).

To verify the robustness of our screening, the inventors selected 4 protective antigens (CSP 09-06, 10-05, 10-10 and 07-03), 4 non-protective antigens (GFP 09-07, 07-05, 07-06 06-06 and 10-06) plus GFP and CSP, and instead of only one immunization dose, the inventors administered one dose of 5×10 TU of non-concentrated VSV^{NJ}B2M LPs and one month later, a second dose of 1×10⁷ TU of VSV^{IND} B2M LPs. As shown in the FIG. 10, the inventors observed three patterns of infection profile when the inventors compared one (circles, data from FIG. 9) and two immunization doses (squares, PB). For the non-protective antigens GFP, 09-07 and 07-05, the second dose of LP did not change the profile of infection, as expected. For the protective antigens CSP (***P<0.001), 09-06, 10-05 and 07-03, the second dose of LP increased the number of protected mice and/or decreased the average parasitemia, also, as expected. Notably, the non-protective antigens 07-06, 06-06 and 10-06, as assessed by one dose of LP immunization, showed a strong protective activity, including a sterile protected mice (PB 7-6), when administered twice in mice.

These results validated some of our protective antigens detected with a single immunization dose, but also showed that some good protective antigens were not detected in our first screen, leading to the decision of repeating the screening using two immunization doses.

### 4. Second Screening of Protective Antigens Using Two Doses of LP

By functionally screening the protective activity of 55 down-selected plasmodial PE antigens using the protocol of two immunization doses, the inventors identified 16 antigens that protected at least one immunized mice per group. Among these 16 antigens, 7 of them (black circles/bars in the FIG. 11) conferred significant protection when compared to animals immunized with the GFP, both when analysing the number of protected mice (Fisher's Exact test) or the mean of the log parasitemia (ANOVA).

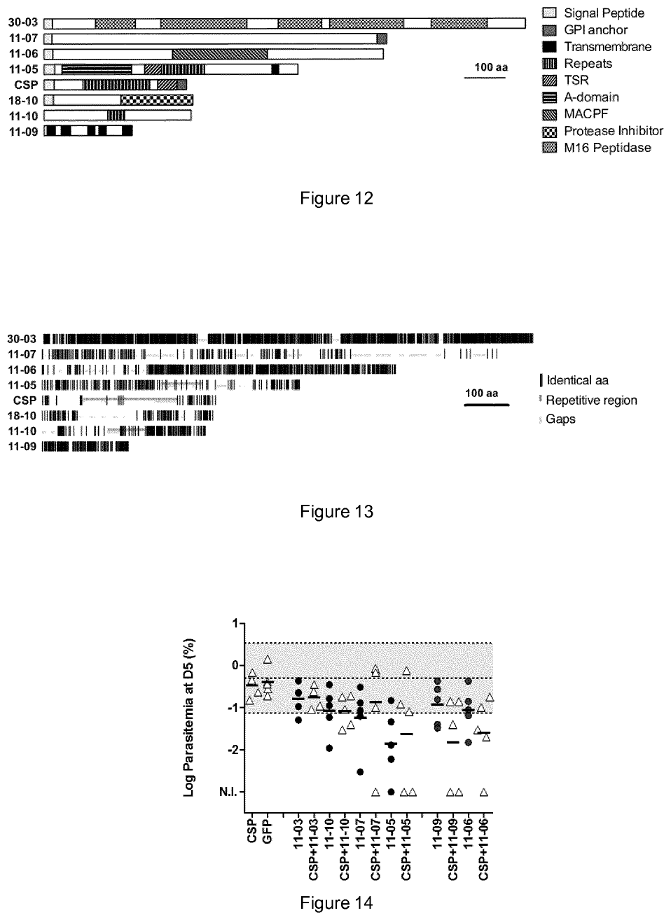

All of them presented a similar or an inferior protective activity when compared individually to our standard of protection, the CSP (red, FIG. 11). Five of them are molecules with assigned function (11-05, 11-06, 11-07, 30-03 and 18-10) and two are proteins with no predicted function (11-09 and 11-10). The structure of these Pb protective antigens is shown in the FIG. 12 and the alignment of these proteins with their respective orthologs from human-infecting parasites, P. falciparum (Pf) and P. vivax (Pv), and macaque-infecting parasite P. cynomolgi, is represented in the FIG. 13. As shown in table I, the percentage of identical amino acids between orthologs varied from 75 to 38% (Pb vs Pf), 78 to 33% (Pb vs Pv) and 79 to 26% (Pf vs Pv). The most conserved genes (>50% identity) are 30-03, 11-09, 11-10 and 11-06 orthologs. Antigens with divergent repetitive sequences are penalized in the alignment by insertional gaps, presenting less percentage of identity.

TABLE I
Percent Identity Matrix created by CLUSTAL 2.1. Amino acid
sequence of Pb antigens were pBlasted against Pf and Pv
taxids (organism) and the best matched sequence was used
to align the orthologous proteins using MUSCLE
(http://www.ebi.ac.uk/Tools/msa/muscle/). The table shows
the percentage of identical amino acids between species.
Raw data is presented in the figures.

| | Amino acid identity (%) | | |
|---|---|---|---|
| Antigen | Pb/Pf | Pb/Pv | Pf/Pv |
| 30-03 | 74.65 | 77.90 | 73 |
| 11-09 | 66.19 | 66.19 | 79.05 |
| 11-06 | 64.92 | 63.40 | 64.44 |
| 11-10 | 56.94 | 50.15 | 62.28 |
| CSP | 42.06 | 33.53 | 26.36 |
| 18-10 | 39.60 | 41.23 | 49.30 |
| 11-05 | 38.75 | 44.67 | 42.86 |
| 11-07 | 37.53 | 33.42 | 46.30 |

In a decreasing order of protection the first antigen identified is TRAP¹⁴ (SEQ ID No 20 and 21) (thrombospondin related anonymous protein; 11-05), which validated our method of screening since immunization with TRAP is known to induce protection both in rodents¹⁵ and humans¹⁶. TRAP is a type I transmembrane protein harbouring two extracellular adhesive domains, a von Willebrand factor type A domain and a thrombospondin type 1 domain, followed by a proline-rich repetitive region. TRAP is stored in micronemal secretory vesicles and following parasite activation, the protein is translocated to the surface of sporozoites where it serves as a linker between a solid substrate and the cytoplasmic motor of sporozoites. Intriguingly, anti-TRAP antibodies do not impair parasite motility and infectivity¹⁷. CD8+ T cells seem to mediate the protection mediated by TRAP immunization^{10,15,16,18}.

The second protective antigen identified is an inhibitor of cysteine protease (ICP, 18-10)¹⁹ (SEQ ID No 29, 30). ICP seems to be involved in the motility and infectivity capacity of sporozoites via the processing of CSP^{20,21}, as well as, in the parasite intra-hepatic development²². Although the protein does not present structural signatures of membrane localization, there is evidence that the protein is located on the surface of sporozoites^{19,20}. Opposing results are published regarding the secretion of the protein following parasite activation^{21,22}. Similarly, there are contradictory results regarding the inhibition of host cell invasion by sporozoites in vitro in the presence of anti-ICP immune sera^{20,23}.

The third protective antigen identified is a metallopeptidase (Falcilysin/Bergheilysin, 30-03)²⁴ (SEQ ID No 38 and 39 for Falcilysin/Bergheilysin A and 47 for Falcilysin/Bergheilysin B). This protease seems to be involved in the catabolism of hemoblobin in the parasite blood stages²⁵. A H-2K^{b}-restricted CD8 T cell epitope was recently described during the parasite blood infection²⁵ suggesting that CD8 T cells could mediate the protection elicited by the antigen 30-03 during the hepatic infection.

The fourth protective antigen is a GPI-anchored protein (P113, 11-07) (SEQ ID No. 57 and 58) initially described in blood stages¹⁶ and also expressed in PE stages. P113 seems to be important for liver infection, dispensable for blood infection, but its precise function is unclear¹⁷.

The fifth antigen is the pore-forming like protein SPECT2 (11-06)²⁸ (SEQ ID No 50). This protein has a membrane attack complex/perforin (MACPF) domain and is involved in the sporozoite cell traversal activity, being important for the progression of sporozoites in the dermis²⁹ and survival to phagocytosis in the liver³⁰.

The sixth antigen identified is a hypothetical protein that the inventors called 11-09 also called Ag40 (SEQ ID No 67 and 68). This protein has 4-5 annotated transmembrane domains. Deletion of the gene coding for the antigen 11-09 caused impairment of Pb parasite development in the liver.

The seventh antigen is also a hypothetical protein that the inventors called 11-10 also called Ag45 (SEQ ID No 76 and 77). This protein doesn't have annotated domains, but possesses a central region with negatively charged amino acids. Recently the 11-10 ortholog of Plasmodium yoelii, another rodent-infecting plasmodial species, was also identified as a protective antigen²¹. The deletion of the gene coding for the antigen 11-blocked the Pb sporozoite invasion of salivary glands and completely abolished the capacity of sporozoites to infect the liver.

To determine if CSP based protection could be additively or synergistically improved by the combination of antigens, the inventors assessed the protection elicited by a sub-optimal dose of CSP (5×10⁵ TU of VSV^{NJ}/5×10⁶ TU of VSV^{IND} B2M LPs) in the absence or presence of an usual dose of protective antigens (5×10⁵ TU of VSV^{NJ}/1×10⁷ TU of VSV^{IND} B2M LPs). This protection induced by CSP+protective antigens was compared to the protection elicited by these antigens alone (data from FIG. 11). As negative control the inventors used animals immunized with the usual dose of GFP LPs. FIG. 14 shows that 4 antigens when combined with a sub-optimal dose of CSP (CSP+11-03, +11-10, +11-07 and +11-05, triangles) did not change the average of protection when compared to the protective activity elicited by these antigens administered alone. In two antigens the antigen combination (CSP+11-09 and CSP+11-06) showed a tendency of better protection (˜10 fold), but it was not statistically significant.

### 5. Identification of Multi-Antigenic Formulations Capable of Sterilizing Sporozoite Infection Via a CD8+ T Cell Response

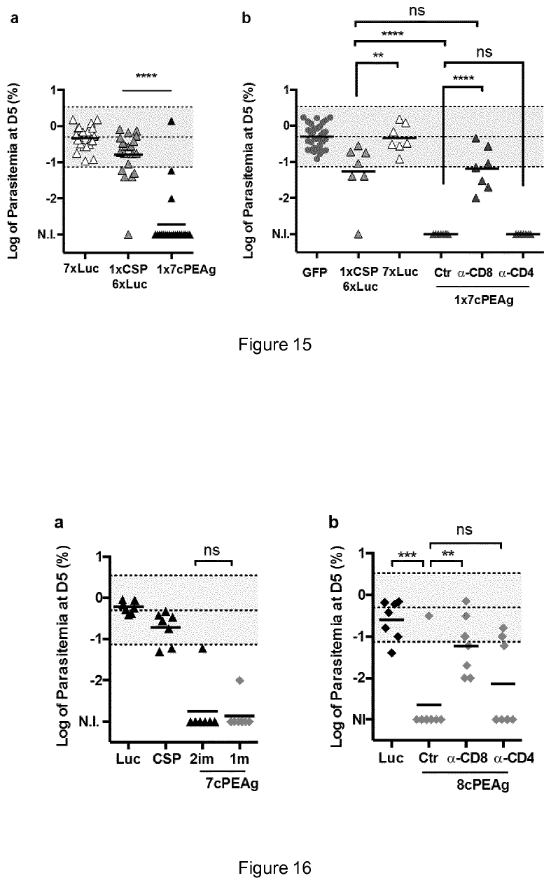

Since testing all possible combinations of antigens was technically unfeasible the inventors decided to evaluate the protection elicited by the combination of these multiple protective antigens. Remarkably, two immunizations of mice with the combination of CSP and 6 of these antigens elicited sterile protection in the vast majority of challenged animals (7PEAg, 86-100%, FIG. 15). This percentage of sterile protection was far superior to the protection conferred by CSP in the same experimental conditions (0-14%). Depletion of CD8+ cells (□-CD8) just before the challenge, but not of CD4+ cells, decreased this protection to the level of that induced by CSP, suggesting that CD8+ T cells and antibodies mediate the extra protection elicited by the addition of these 6 PE antigens.

The same protective efficacy was observed using only a single immunization for the 7PEAg or for the 7PEAg+30-03 (FIGS. 16a and 16b, 8PEAg), as well as, the dependence on CD8+ T cells for the sterilizing immunity of 8PEAg (FIG. 16b). Since the antigen 30-03 is a large molecule and did not improve sterile protection when administered with the 7PEAg, the inventors excluded it from further analysis.

### 6. Design of a Chimeric Antigen Containing the Protective Domains of Down-Selected PE Antigens

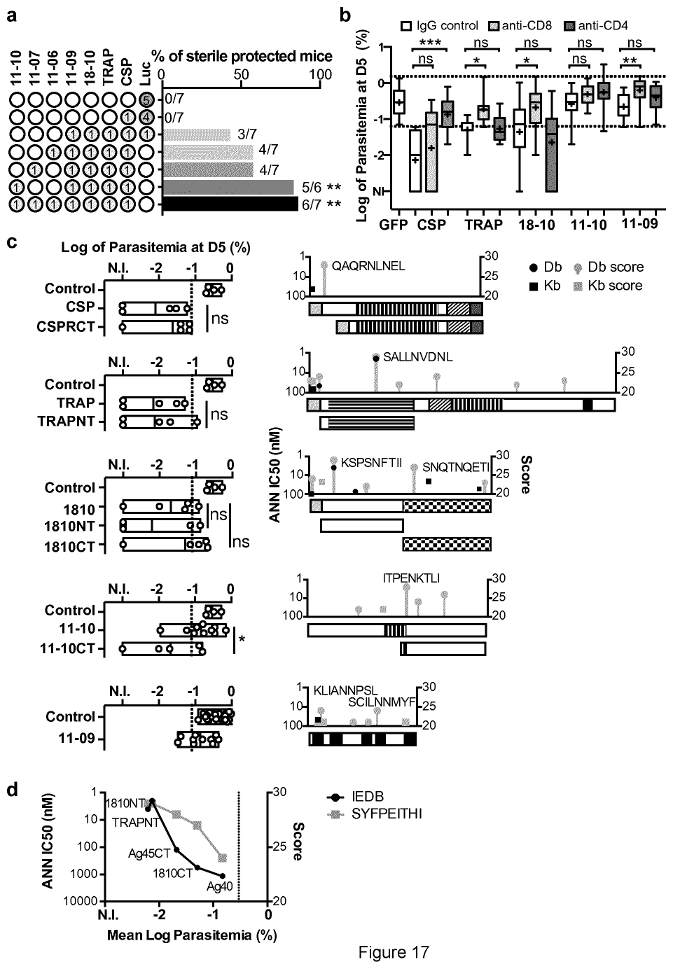

To determine a minimal antigenic composition capable of eliciting this additional protective CD8+ T cell response, the inventors first identified the antigens whose protective activity was dependent on these T cells. Protection induced by two immunizations using TRAP, 18-10 and 11-09 was significantly reduced after depletion of CD8+ cells, as shown in the FIG. 17b. Protection induced by two immunizations using 11-10 was reduced after depletion of CD8+ cells but it was not statistically significant (FIG. 17b). Therefore the inventors grouped CSP with the CD8+ dependent protective antigens, TRAP, 18-10 and 11-09 and added separately 11-10, 11-07 and 11-06 to identify a minimal antigenic combination capable of sterile protect immunized animals like the complete combination of antigens. As shown in FIG. 17a, the combination of 5 antigens, CSP+TRAP, 18-10, Ag40 and Ag45 induced comparable level of sterile protection elicited by the combination of the 7PEAg.

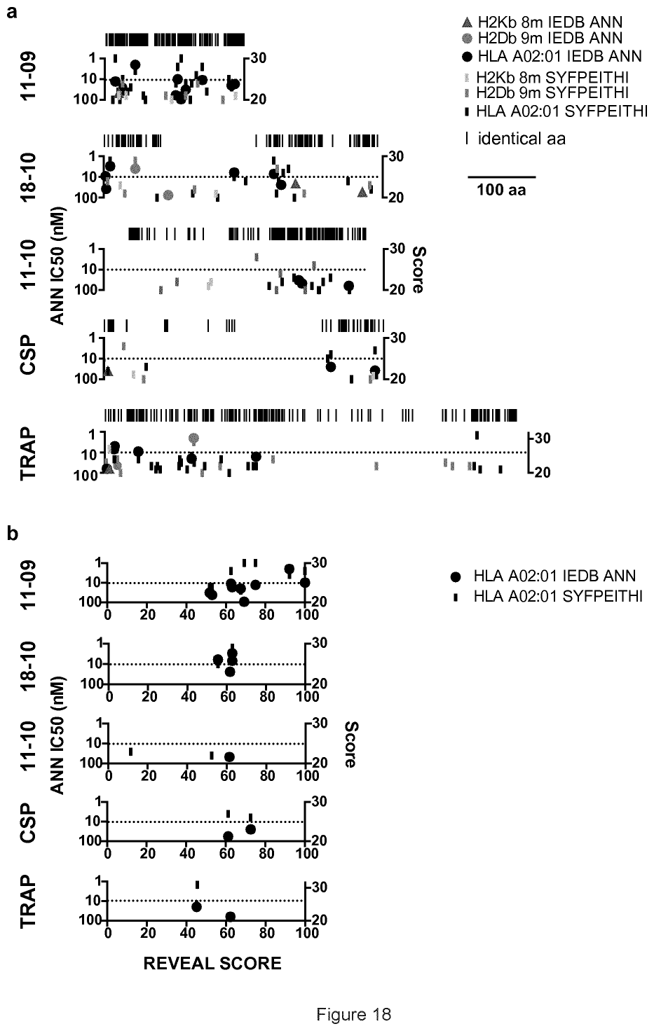

In order to combine the protective domains of each of these 5 antigens in a single chimeric molecule and thus avoid the costs associated with the production and delivery of five different antigens, the inventors mapped the protective regions of each antigen according to the localization of predicted epitopes binding to MHC class I molecules (FIGS. 17c and 18) and structural-functional conserved motifs (FIGS. 12, 13, 17c and 18).

As shown in the FIG. 17c, all tested domains presented either a better (11-10CT) or similar protective activity when compared to the entire antigen. The level of mean protection elicited by the domains of antigens inducing protective CD8+ T cells correlated with the score (P<0.01) or affinity (P=0.01) of CD8+ T cell epitopes respectively predicted by SYFPEITHI and IEDB (FIGS. 17c and 17d). Importantly, the mapping of protective domains allowed the reduction of ˜2000 basepairs in the final chimeric PE antigen construct. Due to its small size, Ag40 was not split in domains and the data presented in the FIG. 17c comes from the experiment showed in the FIG. 11.

Analysis of the distribution of epitopes of Pb antigens predicted to bind to MHC class I molecules of C57BL/6 mice (H2-K^{b}, H2-D^{b}) or of the Pf orthologs predicted to bind to HLA A02:01, a high prevalent human HLA allele, revealed that most of predicted good binders are clustering in the regions that are conserved among different plasmodial species (Pb, Pc, Pv and Pf, FIG. 18). This renders possible to use the Pb protective regions mapped in the FIG. 17 to select the correspondent region in the Pf orthologs.

In addition, the inventors validated the binding of the best predicted Pf epitopes to the HLA A02:01 class I molecule using the REVEAL® binding assay developed by Proimmune, which allows the quantification of the binding and stabilization of the complex formed by the tested peptide, HLA A02:01 and β2-microglobulin (FIG. 18).

In summary, using a parameterized selection of antigens, a screening based on lentiviral vaccination and a direct measurement of protection in vivo against a stringent sporozoite challenge, the inventors identified 8 protective antigens, including the vaccine candidates CSP and TRAP, out of 55 tested antigens. All these 8 antigens are conserved across several plasmodial species. Remarkably, immunization using a combination of seven or eight of these antigens elicited sterile protection in the vast majority of challenged mice, either using one or two immunizations. More importantly, this protection was far superior than the one elicited by CSP, so far the best protective PE antigen. Depletion of CD8+ T cells abolished sterilizing immunity, indicating that these cells are essential for this protective phenotype, similarly to the protection conferred by irradiated sporozoites. A minimal combination of 5 of these antigens was also capable of eliciting sterile protection in most of challenged animals. Mapping of the protective domains of these 5 antigens allowed the design of a chimeric antigen containing the fused protective domains of these 5 down-selected antigens. The human-infecting parasite orthologs of these protective antigens, or of their protective domains are potential candidates for being used in the development of a malaria vaccine formulation containing multiple protective antigens or multiple protective domains fused in a single molecule.

### MATERIAL and METHODS

Parasite Strains:

Plasmodium berghei ANKA strain constitutively expressing the GFP under the control of the hsp70 promoter (Ishino et al, 2006) was used in the challenges using parasitemia, quantified by flow cytometry, as protective readout. Plasmodium berghei ANKA strain constitutively expressing a GFP-luciferase fusion under the control of the eef-1alfa promoter (Franke-Fayard et al, 2008) was used in the challenges using liver infection, assessed by bioluminescence, as protective readout. Of note, parasitemia quantified using hsp70-gfp parasites was at least 10 times more sensible than using eef-la gfp:luc parasites due to more intense expression level of GFP.
Ishino T, Orito Y, Chinzei Y, Yuda M (2006) A calcium-dependent protein kinase regulates Plasmodium ookinete access to the midgut epithelial cell. Mol Microbiol 59:1175-1184.
Franke-Fayard B, Djokovic D, Dooren M W, Ramesar J, Waters A P, et al. (2008) Simple and sensitive antimalarial drug screening in vitro and in vivo using transgenic luciferase expressing Plasmodium berghei parasites. Int J Parasitol 38:1651-1662.

Mouse Strains:

C57BL/6 Rj and Swiss mice were purchased from Elevage Janvier (France). All experiments were approved by the Animal Care and Use Committee of Institut Pasteur (CETEA 2013-0093) and were performed in accordance with European guidelines and regulations (MESR-01324).

Production of Lentiviral Particles Stock:

Down-selected plasmodial antigens were synthesized by Eurofins MWG as mouse codon-optimized genes with the addition of a Kozak consensus sequence (GCCACCATGGCT(C) (SEQ ID No. 85 and 86), representing the first 12 nucleotides in the coding sequences of the antigenic polypeptides), encompassing the first translated ATG. This modification adds an extra alanine after the first methionine. A BamHI (GGATCC—SEQ ID No. 87) and Xho I (CTCGAG—SEQ ID No. 88) restriction sites were also inserted in the 5′ and 3′ extremities of the construct, respectively. These synthetic codon-optimized genes were then cloned into the BamHI and Xho I restriction sites of the pTRIP plasmid harboring either the CMV or B2M promoter (FIGS. 16 and 17). Lentiviral particles were produced by transient calcium co-transfection of HEK 293T cells with three helper plasmids encoding separate packaging functions, the pTRIP vector plasmid containing the synthetic plasmodial gene, the envelope expression plasmid encoding the glycoprotein G from VSV (Vesicular Stomatitis Virus, Indiana (FIG. 19) or New Jersey (FIG. 20) serotypes) and the p8.74 encapsidation plasmid (FIG. 18), containing the structural, accessory and regulatory genes of HIV. This co-transfection will generate integrative but replication-incompetent pseudotyped lentiviral particles. At 24 hours post-transfection, the cell culture medium was replaced by serum-free DMEM. Supernatants were collected at 48 hours post-transfection, clarified by low-speed centrifugation, and stored at −80° C. The lentiviral vector stocks were titrated by real-time PCR on cell lysates from transduced HEK 293T cells and titer were expressed as transduction unit (TU) per ml.

Immunization Protocol:

For the screening using one single dose of LPs, 4 weeks-old C57BL/6 mice (n=5 per group per experiment) were acclimated for 3 weeks and intraperitoneally immunized with a single dose of 1×10⁷ TU of non-concentrated VSV^{IND}B2M LPs. For the protocol using two immunization doses. 4 weeks-old C57BL/6 mice (n=5 per group per experiment) were acclimated for 3 weeks and intraperitoneally immunized with a first dose of 5×10⁵ TU of non-concentrated VSV^{NJ} B2M LPs. Thirty days after the first immunization, the animals received a second dose of 1×10⁷ TU of non-concentrated VSV^{IND} B2M LPs. For testing combinations of a sub-optimal dose of CSP+ an optimal dose of down-selected antigens, mice were immunized twice, four weeks apart, with a sub-optimal dose of CSP (5×10 TU of non-concentrated VSV^{NJ}B2M LP in the first immunization and 5×10⁶ TU of non-concentrated VSV^{IND} B2M LP in the second immunization) and the usual dose of protective plasmodial antigens (5×10⁵ TU of non-concentrated VSV^{NJ} B2M LP in the first immunization and 1×10⁷ TU of non-concentrated VSV^{IND} B2M LP in the second immunization). For testing the combination of multiple antigens, mice were immunized twice, four weeks apart, with 7× the individual dose (1 dose=5×10⁵ TU of non-concentrated VSV^{NJ} B2M LPs in the first immunization/1×10⁷ TU of non-concentrated VSV^{IND} B2M LPs in the second immunization) of the control antigen Al11-luciferase (Luc), with the individual dose of CSP plus 6 doses of Luc or with the individual doses of CSP and of the 6 conserved PE antigens (11-05, 11-06, 11-07, 11-09, 11-10 and 18-10). For this experiment the inventors used ultrafiltration and lenti-X (Clontech) concentrated stocks. The average volume of injection was 500 μL of LPs diluted in DMEM.

In all cases, thirty days after last immunization, mice were challenged with 5,000 GFP-expressing sporozoites micro-injected subcutaneously in the mice footpad.

Sporozoite Challenge:

Anopheles stephensi (Sda500 strain) mosquitoes were reared using standard procedures. 3-5 days after emergence, mosquitoes were fed on infected Swiss mice with a parasitemia superior to 2%, and kept as described in Amino et al, 2007. Between 20 and 23 days post-feeding, the salivary glands of infected mosquitoes were dissected in PBS, collected in 20 μL of sterile PBS on ice and disrupted using an Eppendorf® pestle. The suspension of parasites were filtered through a nylon mesh of 40 um, counted using Kova™ Glasstic™ slide (Hycor) and adjusted to a concentration of 5,000 or 10,000 sporozoites/μL with sterile PBS. This suspension was divided in individual tubes, one for each group of immunized mice (n=4-7 per group), and kept on ice until the challenge. One microliter of parasite suspension was injected in the right footpad of mice using a Nanofil syringe (World Precision Instruments) with a 35 GA bevelled needle (NF35BV).

Amino R, Thiberge S, Blazquez S, Baldacci P, Renaud O, et al. (2007) Imaging malaria sporozoites in the dermis of the mammalian host. Nat Protoc 2:1705-1712.

Measurement of Parasite Infection:

Hepatic parasite loads were quantified at ˜44h by bioluminescence in fur shaved mice infected with GFP LUC parasites. Infected mice were first anesthetized with isoflurane and injected subcutaneously with D-luciferin (150 mg/kg, Caliper LifeSciences). After a 5 minutes incubation allowing the distribution of the substrate in the body of the anesthetized animals, mice were transferred to the stage of an intensified charge-coupled device photon-counting video camera box where anesthesia was maintained with 2.5% isoflurane delivered via nose cones. After 5 minutes of signal acquisition controlled by the Living Image software (Xenogen Corporation), animals were returned to their cage. Automated detection of bioluminescence signals by the system resulted in the generation of bioluminescence signal maps superimposed to the gray-scale photograph of the experimental mice. These images were then quantified using the Living Image software. Briefly, regions of interest (ROI) encompassing the liver were manually defined, applied to all animals and the average radiance within these ROIs was automatically calculated. Background signal was measured in the lower region of the abdomen, and the average values of background signal obtained.

Alternatively, blood infection was assessed by flow cytometry using hsp70-GFP parasites. At day 4, 5, 6 and >10 post-challenge, a millimetric excision was performed in the tail of mice allowing the collection of a drop of blood that was readily diluted in 500 μL of PBS. This diluted blood was analyzed using a flow cytometer. 500,000 events were analyzed at day 4 post-challenge and 100,000 events in the subsequent days. Non-infected mice after day 10 were considered as sterile protected.

Statistical Analysis:

Parasitemia data from GFP immunized control were log transformed and pooled for the calculation of 95% tolerance of interval with 95% of certitude. For the immunization protocol of one dose this limit comprised the interval of the mean value ±2.49 SD (mean=−0.3906, SD=0.3392, n=35). Similarly, for the immunization protocol of two doses this limit comprised the interval of the mean value ±2.51 (mean=−0.3002, SD=0.3305, n=33). All mice with a log parasitemia inferior to the lower limit (mean−2.5 SD) were considered as significantly different from the control mice (P<0.05), and therefore considered as protected. In the protocol using two immunization doses, the difference in the numbers of protected mice, following the definition above, between the test group and the GFP control group was assessed using the Fisher's exact test. The average of the log parasitemia of the groups with significant differences in the Fisher's Test were compared to the GFP group using one-way ANOVA (Holm-Sidak's multiple comparison test).

### REFERENCES

1. http://www.who.int/mediacentre/factsheets/fs094/en/
2. RTS,S Clinical Trials Partnership. Lancet. 2015; 4; 386(9988):31-45.
3. Moorthy V S, Ballou W R. Malar J. 2009; 8: 312.
4. http://www.who.int/immunization/topics/malarialvaccine_roadmap/en/
5. Seder R A, et al. Science. 2013; 341:1359-65.
6. Amino R, Ménard R. Nature. 2012; 484(7395):S22-3
7. Kester K E, et al. 2014. pii: S0264-410X(14)00822-6.
8. Mishra S, et al. Vaccine. 2011; 29(43):7335-42.
9. Murphy S C, Kas A, Stone B C, Bevan M J. Proc Natl Acad Sci USA. 2013; 110(15):6055-60.
10. Hafalla J C, et al. PLoS Pathog. 2013; 9(5):e1003303.
11. Iglesias, M. C., et al. J Gene Med. 2006; 8, 265-274.
12. Firat, H., et al. J Gene Med. 2002; 4, 38-45.
13. Doolan D L, et al. Proc Natl Acad Sci USA. 2003; 100:9952-7.
14. Robson K J, et al. Nature. 1988; 335(6185):79-82.
15. Khusmith S, et al. Science. 1991; 252(5006):715-8.
16. Ewer K J, et al. Nat Commun. 2013; 4:2836.
17. Gantt S, et al. Infect Immun. 2000; 68(6):3667-73.
18. Khusmith S, Sedegah M, Hoffman S L. Infect Immun. 1994; 62(7):2979-83.
19. LaCrue A N, et al. Mol Biochem Parasitol. 2006; 148(2):199-209.
20. Rennenberg A et al. PLoS Pathog. 2010; 6(3):e1000825.
21. Boysen K E, Matuschewski K. MBio. 2013; 4(6):e00874-13.
22. Lehmann C, et al. PLoS Pathog. 2014; 10(8):e1004336.
23. Pei Y, et al. Cell Microbiol. 2013 September; 15(9):1508-26.
24. Eggleson K K, Duffin K L, Goldberg D E. J Biol Chem.; 274(45):32411-7.
25. Poh C M, Howland S W, Grotenbreg G M, Rénia L. Infect Immun. 2014; 82(11):4854-64.
26. Gilson P R, et al. Mol Cell Proteomics. 2006; 5(7):1286-99.
27. Offeddu V, Rauch M, Silvie O, Matuschewski K. Mol Biochem Parasitol. 2014; 193(2):101-9.
28. Ishino T, Chinzei Y, Yuda M. Cell Microbiol. 2005; 7(2):199-208.
29. Amino R, et al. Cell Host Microbe. 2008 Feb. 14; 3(2):88-96.
30. Tavares J, et al. J Exp Med. 2013; 210(5):905-15.
31. Speake C, et al. PLoS One. 2016; 11(7):e0159449.

1-21. (canceled)

22. A method of functionally screening protective antigenic polypeptides of a determined pathogen or cancer cell comprising the steps of:
a. optionally pre-selecting candidate antigenic polypeptides for the screening by reference to a group of genes, transcripts or proteins identified for a determined pathogen or cancer cell wherein the antigenic polypeptides may constitute putative targets for a humoral and/or cellular immune response,
b. providing a lentiviral vector, in particular a HIV-1 based vector, expressing one or more antigenic polypeptide(s) to be assayed for its (their) immunogenic properties,
c. immunizing a non-human mammal in particular a non-human mammal model selected for its susceptibility to infection by the determined pathogen or susceptibility to cancer development, with the lentiviral vector of step b. in immunization dose conditions enabling elicitation of a potent cellular and/or humoral memory response against the antigenic polypeptide(s),
d. challenging the immunized non-human mammal of step c. with the pathogen or administering cancer cells to the immunized non-human mammal of step c. and quantifying the development of the pathogen or the cancer cell in the non-human mammal thereby enabling functional identification of the protective response capacity of the antigenic polypeptide(s).

23. A method according to claim 22 wherein the pathogen is selected in the group of extracellular or intracellular viruses, bacteria, fungi, protozoans and worms.

24. A method according to claim 23 wherein the pathogen is selected among arboviruses, hemorrhagic-fever causing viruses, immunodeficiency-causing viruses, Mycobacterium spp, Yersinia spp, Listeria ssp, Histoplasma spp, Cryptococcus spp, kinetoplastida parasites, apicomplexan parasites, and cestode, trematode or nematode worms

25. A method according to claim 22 wherein the antigenic polypeptide(s) are cancer cell antigens selected in the group of melanoma, breast carcinoma, B cell lymphoma, colon cancer in particular colon carcinoma, liver cancer, lung cancer, bladder cancer in particular bladder carcinoma, mastocytoma, pancreas cancer and prostate adenocarcinoma.

26. A method of functionally screening immunogenic properties of antigenic polypeptides according to claim 22 wherein the pathogen is a determined Plasmodium parasite comprising the steps of:
a. optionally pre-selecting candidate antigenic polypeptides for the screening by reference to a group of genes, transcripts or proteins identified for a determined Plasmodium parasite wherein the group may reflect a particular stage of the development of the parasite or a particular biological function,
b. providing a lentiviral vector, in particular a HIV-1 based vector, expressing one or more antigenic polypeptide(s) to be assayed for its protective properties,
c. immunizing a non-human mammal model selected for its susceptibility to infection by the determined Plasmodium parasite with the lentiviral vector of step b. in immunization dose conditions enabling elicitation of a potent cellular and/or humoral memory response against the antigenic polypeptide,
d. challenging the immunized non-human mammal of step c. with sporozoites of the Plasmodium parasite and quantifying the development of the Plasmodium parasite in the non-human mammal thereby enabling functional identification of the protective response capacity of the antigenic polypeptides.

27. The method according to claim 26, wherein the Plasmodium parasite is P. berghei, or P. yoelii and the non-human mammal model is a rodent, in particular a mouse susceptible to said Plasmodium parasite, especially a C57Bl/6 mouse for P. berghei, or a BALB/c mouse for P. yoelii.

28. The method according to claim 22 wherein the non-human mammal model is a rodent, in particular is a rodent selected as follows: for melanoma: B16F10 Bl6 mice, for breast carcinoma: 4T1 BALB/c mice, for B cell lymphoma: A20 BALB/c mice, for bladder cancer MBT2 C3H/HeN mice, for bladder carcinoma: AY27 Fischer rats, for colon cancer ProB BD9 rats, for colon carcinoma: CT26 Balb/C mice, for liver cancer Hepa1-6 C57 mice, for lung cancer LL/2 C57Bl6 mice or TC1 C57BL mice, for mastocytoma: P815 DBA/2 mice, for pancreas cancer: PAN02 C57BL/6 mice, for prostate Adenocarcinoma: R3327H Cop Rat.

29. The method of claim 22 wherein the immunization comprises administering one dose of lentiviral vector in a priming step and one dose of lentiviral vector in a boosting step wherein in the priming and boosting steps the lentiviral vectors are pseudotyped with different non cross-seroneutralizing envelope proteins and wherein the priming and the boosting doses are different, in particular the boosting dose is higher than the priming dose or wherein the priming and boosting doses are equal.

30. The method according to claim 22, wherein the administered doses of lentiviral vector expressing the antigenic polypeptide for immunization of the non-human mammal are each in the range of 1×10⁵ to 1×10⁸ TU, in particular 1×10⁵ to 1×10⁷ and wherein the lentiviral vector is formulated as a suspension of either concentrated or non-concentrated lentiviral vector.

31. The method according to claim 22, wherein the challenge of the non-human mammal is carried out by administration to said non-human mammal of Plasmodium parasites, pathogens or cancer cells expressing a constitutive bioluminescent or a fluorescent marker.

32. The method according to claim 26, wherein the quantification of the protective response against the antigenic polypeptide comprises a step of quantifying the load of the sporozoites expressing a constitutive bioluminescent marker, in the liver of the non-human mammal, especially by bioluminescence.

33. The method according to claim 26 wherein the quantification of the protective response against the antigenic polypeptide comprises a step of quantifying the growth of the Plasmodium parasite, in particular of Plasmodium parasites expressing a constitutive fluorescent marker, on red blood cells harvested from a blood sample of the non-human mammal, especially by flow cytometry.

34. The method of claim 26 wherein a positive control for protection capability against infection by Plasmodium parasite or against the parasite-induced condition or disease is used which is CSP, or an antigen selected among antigens on the gametes, zygotes or ookinetes of the parasite such as sexual-stage antigens, antigens of the liver-stage or antigens of the asexual blood-stage, such as antigens of sporozoites, in particular an antigen selected among P48/P45 antigen Pfs25, MSP1, CSP, TRAP, Celtos, SPECT, ICP, and Facilysin/Bergheilysin Ag11-09, Ag11-10 and TRAP and a negative control for protective capability is used, which is GFP, a known non-protective antigen or an empty equivalent vector.

35. The method according to claim 22, wherein immune-sera in the sample obtained from immunized non-human mammal were incubated with GFP-expressing Plasmodium sporozoites wherein a subgroup of these sporozoites is permeabilzed and a subgroup of these sporozoites is not permeabilized.

36. The method according to claim 22, wherein the lentiviral vector contains in its genome a synthetic gene for an antigenic polypeptide of Plasmodium the coding sequence of which is codon-optimized for the expression of the antigenic polypeptide in mammalian cells and wherein expression of the antigenic polypeptide is driven by a promoter suitable for directing gene expression in various mammalian cell types, including dendritic cells, such as a beta-2 microglobulin promoter.

37. The method according to claim 22, wherein the genome of the lentiviral vector is obtained from a transfer vector which is a pTRIP plasmid wherein a Plasmodium synthetic nucleic acid encoding the antigenic polypeptide to be assayed has been cloned under control of a promoter functional in mammalian cells, in particular the human beta-2 microglobulin promoter, and optionally under the control of post-transcriptional regulatory element of the woodchuck hepatitis virus (WPRE).

38. The method according to claim 37, wherein the synthetic nucleic acid is a mammal-codon optimized nucleic acid encoding the antigenic polypeptide to be assayed.

39. The method according to claim 22, wherein the envelope protein pseudotyping the lentiviral vector is a VSV-G envelope protein from the Indiana strain, from the New Jersey strain or from any other non-cross neutralizing strain, such as Cocal and Chandipura.

40. The method of claim 22, wherein the pre-selection of candidate antigenic polypeptide includes the step of transcriptome profiling of the parasite for the pre-erythrocytic stage of development compared with transcriptome profiling of the parasite for a different stage of development, in particular the blood-stage of development of said parasite and identifying over expressed transcripts in the pre-erythrocytic stage parasite.

41. A method of identifying an antigenic polypeptide for the design of a vaccine candidate against malaria parasite for protection of a human host, which comprises the steps of screening antigenic polypeptides according to a method of claim 22, and determining the orthologous gene in a Plasmodium parasite infecting a human host, in particular Plasmodium falciparum, Plasmodium vivax, Plasmodium malariae, Plasmodium ovale or Plasmodium knowlesi.
